(19) United States
(12) Patent Application Publication
(10) Pub. No.: US 2023/0148001 A1
(43) Pub. Date: May 11, 2023

(54) TRANSFORMED PLANTS AND METHODS FOR MAKING AND USING THE SAME

(71) Applicants: Foley L.S. MARQUESS, Saskatoon (CA); Paul J. FOOR, Canyon, TX (US); Relica Genomics Inc., Saskatoon (CA)

(72) Inventors: Foley L.S. Marquess, Saskatoon (CA); Paul J. Defoor, Canyon, TX (US)

(21) Appl. No.: 17/638,446

(22) PCT Filed: Aug. 27, 2020
(86) PCT No.: PCT/US2020/048294

§ 371 Date: Feb. 25, 2022

Related U.S. Application Data
(60) Provisional application No. 62/892,219, filed on Aug. 27, 2019.

(51) Int. Cl. C12N 15/82; A01H 6/46; A61K 39/114

(52) U.S. Cl. CPC C12N 15/8214; A01H 6/46; A61K 39/114; A61K 2039/552

(57) ABSTRACT

The present invention is directed to a transformed plant and a method for producing and using the same, or components thereof.

### CROSS REFERENCE TO RELATED APPLICATIONS

This application claims the benefit of U.S. Provisional Application No. 62/892,219, filed Aug. 27, 2019, the contents of which is hereby incorporated by reference.

### BACKGROUND OF THE INVENTION

Ruminant infections resulting from Fusobacterium infestations seriously impacts animal production and performance, and leukotoxin A (ltkA) protein is considered to be the major virulence factor in the development of liver abscesses and foot rot in beef and dairy cattle (Narayanan et al., 2001). Methods of treating and preventing this and other infections in cattle remains a costly challenge in the livestock industry.

### SUMMARY OF THE INVENTION

The present disclosure provides, among other things, methods of transforming a plant and compositions including transformed plants or portions thereof (e.g., an antigenic protein or fragment thereof produced by a plant encompassed in the present disclosure). In one aspect of the disclosure, methods of transforming a plant include providing a nucleic acid material and transforming a chloroplast in a plant cell with the nucleic acid material. In some embodiments, a nucleic acid material includes a first targeting sequence and a second targeting sequence, a promoter sequence, and an exogenous nucleic acid sequence. In some embodiments, a nucleic acid material also includes one or more additional components such as a selection sequence and/or an enhancer sequence.

In one aspect of the disclosure, methods of transforming a plant include providing a nucleic acid material and a carrier, and transforming a chloroplast in a plant cell with the nucleic acid material. In some embodiments, methods of transforming a plant further include expressing an exogenous nucleic acid sequence, where the expression occurs, at least in part, in a chloroplast. In some embodiments, an exogenous nucleic acid sequence is transiently expressed in the chloroplast of the plant cell. In some embodiments, an exogenous nucleic acid sequence is integrated in the chloroplast genome of the plant cell. In some embodiments, an exogenous nucleic acid sequence is stably integrated in the chloroplast genome of the plant cell.

In some embodiments, transforming a plant is or comprises transduction. In some embodiments, after a plant cell has been transformed with a nucleic acid material, a portion of the nucleic acid material is removed. The portion of the nucleic acid material that is removed could, for example, be a selection marker. Removal of a portion of nucleic acid material could be through homologous recombination and/or site-specific recombination, for example, cre-lox recombination.

In accordance with various embodiments, a nucleic acid material may be conjugated to a carrier, such as a nanoparticle, and transforming a chloroplast may comprise contacting a plant cell with the nanoparticle conjugated to the nucleic acid material. In some embodiments, a carrier is a nanoparticle. A nanoparticle may be or include, for example, a nanotube. In some embodiments, a nanotube comprises a single-walled nanotube. In some embodiments, a nanotube is a carbon nanotube.

As is described in the present disclosure, a variety of plants to be transformed with nucleic acid material are contemplated. In some embodiments, a plant is millet or sorghum. In some embodiments comprising a plant transformed with nucleic acid material as described herein, the plant is able to express the exogenous nucleic acid sequence, at least in part, in the chloroplast of the plant.

Another aspect of the disclosure includes a kit that includes a nucleic acid material comprising a first targeting sequence and a second targeting sequence, a promoter sequence, a selection sequence; and at least one exogenous nucleic acid sequence; and a nanoparticle carrier. In some embodiments, a nanoparticle carrier may be any known nanoparticle, nanoparticle composition, or nanotube (e.g., as described elsewhere herein).

As is described in the present disclosure, an exogenous nucleic acid material, in some embodiments, is or comprises a RNA oligonucleotide, a DNA oligonucleotide, a plasmid, and any combination thereof. In some embodiments, an exogenous nucleic acid can include two or more exogenous nucleic acid sequences.

In some embodiments, a nucleic acid material includes a first targeting sequence and a second targeting sequence, a promoter sequence, and an exogenous nucleic acid sequence. In accordance with various embodiments, a promoter sequence is selected from PpsbA, Prrn, Prna, psaA, PrbcL, CaMV35S, rbcS, and any combination thereof. In some embodiments, a first and second targeting sequence each have sequences at least 80% identical to SEQ ID NO: 15 and SEQ ID NO: 16, respectively.

In accordance with various embodiments, at least one of a first targeting sequence and a second targeting sequence are directed to sequences located between chromosomal coordinates selected from trnl-trnA, trnM-trnG, rrn16-rps12/7, tscA-psac, trnV-trnA, rbcL-accD, rp132-trnL, 3′rps12/7-trnV, petA-psbJ, Trn16/V-16srrnA, trnfM-trnG, atpB-rbcL, trN-trnR, Ycf3-trnS, Rps7-ndhB, trnY-GUA-trnD-GUC, trnG-UCC-trnM-CAU, trnT-trnL and any combination thereof. In some embodiments, a first targeting sequence and a second targeting sequence are directed to a sequence located between trnG-UCC-trnM-CAU. In some embodiments, a first targeting sequence and a second targeting sequence are directed to a sequence located between trnY-GUA-trnD-GUC. In some embodiments, a first targeting sequence and a second targeting sequence are directed to a sequence located between trnT-trnL. In some embodiments, a first targeting sequence and a second targeting sequence each have sequences at least 80% identical to SEQ ID NO: 1 (bases 14048-14793 of the sorghum chloroplast genome) and SEQ ID NO: 8 or 23, respectively.

In accordance with various embodiments, an exogenous nucleic acid sequence can include any nucleic acid that is non-native to the plant cell being transformed as described herein. In some embodiments, an exogenous nucleic acid sequence encodes a peptide comprising a sequence that is at least 80% identical to a leukotoxin A (ltkA) protein according to Genbank: DQ672338, or a fragment or variant thereof. In some embodiments, an exogenous nucleic acid sequence comprises a sequence encoding at least one region of ltkA selected from the group consisting of PL1, PL2, PL3, PL4, PL5, or a fragment or variant thereof.

In some embodiments, a nucleic acid material may also include one or more additional components such as a selection sequence, enhancer sequence, and/or termination sequence. In some embodiments, a selection sequence may include at least one antibiotic selection sequence. Examples of antibiotic selection sequences include, without limitation, a nucleic acid sequence encoding a spectinomycin resistance gene, a streptomycin resistance gene, a Kanamycin resistance gene, a gentamycin resistance gene, a neomycin resistance gene, a Beta lactam resistance gene, and any combination thereof.

In some embodiments, a selection sequence can include a nucleic acid sequence encoding: a His tag, GUS uidA lacz, green fluorescent protein, yellow fluorescent protein, red fluorescent protein, cyan fluorescent protein, and any combination thereof. Examples, without limitation, include yellow fluorescent protein (YFP, GenBank: GQ221700.1), red fluorescent protein (DsRED, GenBank: KY426960.1), or cyan fluorescent protein (CFP, GenBank: HQ993060.1).

In some embodiments, an enhancer sequence included in the nucleic acid material is selected from one or more of a sequence encoding: ggagg, rrn 5′UTR, T7gene10 5′ UTR, LrbcL 5′UTR, LatpB 5′UTR, Tobacco mosaic virus omega prime 5′UTR (GenBank: KM507060.1), Lcry9Aa2 5′UTR, atpI 5′UTR, psbA 5′UTR, cry2a, rrnB, rps16, petD, psbA, pabA, and any combination thereof.

In some embodiments, a termination sequence comprises a sequence encoding rps16 (GenBank: MF580999.1) or a portion or fragment thereof.

In some embodiments, the present disclosure provides method of administering a modified plant comprising an antigen to a non-human animal, the methods including administering an immunogenic composition including a modified plant to a non-human animal. Administering a modified plant, in some embodiments, can include feeding the modified plant to the non-human animal. In some embodiments, a non-human animal is selected from a cow, a goat, and a chicken.

In some embodiments, a non-human animal is fed an immunogenic composition comprising a modified plant for an extended period of time. In some embodiments, a non-human animal is fed an immunogenic composition for a period of greater than 1, 2, 3, 4, 5, 6, or 7 days (e.g., consecutive days). In some embodiments, a non-human animal is fed an immunogenic composition for a period of greater than 1, 2, 3 4, 5, 6, 7, 8, 9, 10, 11, or 12 weeks (e.g., consecutive weeks). In some embodiments, a non-human animal is fed an immunogenic composition daily. In some embodiments, a non-human animal is fed an immunogenic composition weekly.

Another aspect of the disclosure includes a method of treating one or more symptoms of Fusobacterium infection in a non-human animal. In some embodiments, the method comprises administering an immunogenic composition, wherein the immunogenic composition comprises a plant comprising an exogenous nucleic acid sequence, wherein at least one exogenous nucleic acid sequence is expressed, at least in part, in the chloroplast of the plant. In some embodiments, the exogenous nucleic acid sequence encodes a peptide comprising sequence that is at least 80% identical to a leukotoxin A (ltkA) protein) according to GenBank Ref: DQ672338, or a fragment or variant thereof.

In some embodiments, the one or more symptoms of Fusobacterium infection include foot rot and/or liver abscess. In some embodiments, the immunogenic composition comprises an amount of peptide at least 80% identical to ltkA protein is at least about 0.5% of the total soluble protein in the immunogenic composition.

In some embodiments, the non-human animal does not show significant progression of disease or shows slower progression of disease compared to a control after 28 days of administration. In some embodiments, the non-human animal exhibits delayed onset of symptoms or reduced severity of symptoms of an Fusobacterium infection, compared to a control. In some embodiments, the symptom is footrot, wherein the symptom is characterized by one or more of painful inflammation of the interdigital skin of the infected animal, lameness, loss of appetite, loss of weight, and mortality.

In some embodiments, the administering is or comprises feeding. In some embodiments, the non-human animal is selected from a cow, a goat, and a chicken.

In some embodiments, the non-human animal is fed the immunogenic composition for an extended period of time. In some embodiments, the non-human animal is fed the immunogenic composition for a period of greater than 1, 2, 3, 4, 5, 6, 7 days (e.g., consecutive days). In some embodiments, the non-human animal is fed the immunogenic composition for a period of greater than 1, 2, 3 4, 5, 6, 7, 8, 9, 10, 11, or 12 weeks (e.g., consecutive weeks). In some embodiments, the non-human animal is fed the immunogenic composition daily. In some embodiments, the non-human animal is fed immunogenic composition weekly. In some embodiments, the non-human animal is fed immunogenic composition continuously.

In some embodiments, the plant is millet or sorghum. In some embodiments, the exogenous nucleic acid sequence comprises a sequence encoding at least one region of ltkA selected from the group consisting of PL1, PL2, PL3, PL4, PL5, or any a fragment or variant thereof.

Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

The Figures described below, which together make up the Drawing, are for illustration purposes only, not for limitation.

FIG. 1 shows an example DNA construct for transformation into a sorghum chloroplast genome.

FIG. 2 shows an example DNA construct for transformation into a sorghum chloroplast genome.

FIG. 3 shows an example DNA construct for transformation into a sorghum chloroplast genome.

FIG. 4 shows an example DNA construct for transformation into a millet chloroplast genome.

FIG. 5 shows gel images of PCR products amplified from plasmid templates using respective sorghum and millet flanking primers. Plasmid PCR products are loaded in technical replicates. Lane 1: 1.5 kb ladder. Lanes 2 and 3: millet PL1+PL4 plasmid amplified with millet flanking primers; expected size is 5385 bases. Lanes 4 and 5: sorghum PL1 plasmid amplified with sorghum flanking primers; expected size is 4022 bases. Lanes 6 and 7: sorghum PL4 plasmid amplified with sorghum flanking primers; expected size is 4607 bases. Lanes 8 and 9: sorghum PL1+PL4 plasmid amplified with sorghum flanking primers; expected size is 5069 bases.

FIG. 6 shows Sybr PCR detection data from five sorghum plants two days after inoculation with PL1 construct. DNA was extracted from sorghum plants two days post inoculation. Amplifications with PL1-specific oligos, and no template control (NTC).

FIG. 7 shows Sybr PCR detection data from five sorghum plants two days after inoculation with PL4 construct. DNAs were extracted from sorghum plants two days post inoculation. Amplifications with PL4-specific oligos, and NTC.

FIG. 8 shows Sybr PCR detection data from five sorghum plants two days after inoculation with PL1+PL4 construct. DNAs were extracted from sorghum plants two days post inoculation. Panel (A) shows: amplifications with PL1-specific oligos and NTCs NRTs of are indicated. Panel (B) shows: amplifications with PL4-specific oligos and NTCs are indicated.

FIG. 9 shows reverse transcriptase Sybr PCRs of PP2a reference genes from cDNAs generated from millet (panel A) and sorghum (panel B) plants in this study. PP2a (sorghum/millet serine/threonine-protein phosphatase) and NTCs and no reverse transcriptase (NRT; i.e. RNA) are indicated.

FIG. 10 shows expression (i.e., evidence for recombination of transgenic construct) in sorghum. Panel (A) Reverse transcriptase Sybr PCRs of PL1 and PL4 from cDNAs generated from all sorghum plants in this study. NTCs are indicated. Panel (B) Agarose gel stains of amplicons generated from PCR primers located outside the left side the construct (i.e. on the native chloroplast genome) and inside the insert (i.e. F. necrophorum PL1; left gel), and outside the right side of the construct and inside the insert (right gel), with total DNA prepared from PL1+PL4 inoculated sorghum. Left gel lane 1: high mass ladder; lane 2: PCR amplicon; lane 3: 1 kb ladder. Right gel lane 1: PCR amplicon; lane 2: 1 kb ladder. The schematic below the gels illustrates the targeted locations responsible for the respective left and right gel amplicons. Thin green hoop represents the circular sorghum chloroplast genome; thick green lines represent the construct flanking regions, which are indistinguishable from chloroplast DNA; blue arrows represent relative primer locations; black lines represent relative amplification targets; thick yellow line is transgenic material that includes F. necrophorum PL1, PL4, and associated genetic expression mechanisms and reference genes.

FIG. 11 shows a Clustalw alignment of PL4 PCR product sequence in FIG. 6 (PL4_RTPCRprod) and the sequence of the expected PL4 coding DNA region (PL4_DNAseq). Primer sequences were removed from the alignment.

FIG. 12 shows an agarose gel stain of amplicon generated from PCR primers located outside the left side of the construct and inside the insert. Left lane: 1 kb ladder, right lane: PCR amplicon. The schematic below the gel illustrates the targeted location responsible for the gel amplicon. Thin green hoop represents the circular sorghum chloroplast genome; the thick green lines represent the construct flanking regions, which are indistinguishable from chloroplast DNA; the blue arrows represent relative primer locations; the black line represents amplification target.

FIG. 13 shows expression of the transgenic construct in sorghum. Reverse transcriptase quantitative Taqman PCRs (RT-qPCRs) derived from mRNAs collected from sorghum plants two days post-inoculation. Reference gene PP2a, immunogenic subunit of Fusobacterium leukotoxin (PL4), and no reverse transcriptase (i.e. RNA) controls are indicated.

FIG. 14 shows evidence of homologous recombination of the transgenic construct and the millet chloroplast genome. Agarose gel stains of amplicons generated from PCR primers located outside the left side the millet construct (i.e. on the native chloroplast genome) and inside the insert (i.e. F. necrophorum PL1) with total DNA prepared from PL1+PL4 inoculated millet. Left lane: PCR amplicon; right lane: 1 kb ladder. The schematic illustrates the targeted locations responsible for the respective left and right gel amplicons. Thin blue hoop represents the circular millet chloroplast genome; thick blue lines represent the construct flanking regions, which are indistinguishable from chloroplast DNA; blue arrows represent relative primer locations; black lines represent relative amplification targets; and the thick yellow line is transgenic material that includes F. necrophorum PL1, PL4, and associated genetic expression mechanisms and reference genes.

FIG. 15 shows expression of the transgenic construct (PL1 and PL4) in millet. Reverse transcriptase quantitative PCRs (RT-qPCRs) derived from mRNAs collected from inoculated millet plant, No reverse transcriptase (i.e. RNA) controls (NRTs) and no template controls (NTCs) are indicated.

FIG. 16 shows PCRs from 20 sorghum plants three months after inoculation with PL1, PL4, and PL1+PL4 constructs. Amplifications with PL1- and PL4-specific assays were used to detect presence of their respective construct, along with PP2a to detect presence of sorghum genome. No template controls (NTCs) are also shown.

FIG. 17 shows a Clustalw alignment of PL1 PCR product sequence in FIG. 12 (top) and the sequence of the expected PL1 coding DNA region (bottom). Primer sequences were removed from the alignment.

FIG. 18 shows a Clustalw alignment of PL4 PCR product sequence in FIG. 12 (top) and the sequence of the expected PL4 coding DNA region (bottom). Primer sequences were removed from the alignment.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

About: The term “about” or “approximately”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system (e.g., a non-human animal). Those of ordinary skill in the art will be aware of a variety of routes that may, in appropriate circumstances, be utilized for administration to a subject, for example a human or a non-human. If, for example, in some embodiments, administration may be ocular, oral, parenteral, topical, etc. In some particular embodiments, administration may comprises feeding a composition to a non-human animal. In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time). In some embodiments, administration may involve continuous dosing (e.g., perfusion) for at least a selected period of time. In some particular embodiments, an animal may be fed a composition in a dosing regimen that is intermittent (e.g., a plurality of doses separated in time) and/or periodic (e.g., individual doses separated by a common period of time) dosing. In some particular embodiments, an animal may be fed a composition continually over a period of time.

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. In some instances, as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term may refer to a compound or entity that lacks or is substantially free of any polymeric moiety.

Amelioration: as used herein, refers to the prevention, reduction or palliation of a state, or improvement of the state of a subject. Amelioration includes, but does not require, complete recovery or complete prevention of a disease, disorder or condition (e.g., an infectious disease).

Animal: As used herein refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, chicken, goat, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Antigen: The term “antigen”, as used herein, refers to an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an antigen elicits a cellular response (e.g., involving T-cells whose receptors specifically interact with the antigen). In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen.

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Breed: As used herein, the term “breed” refers to a group of animals (e.g., cattle) having common ancestors and/or sharing certain distinguishable traits that are not shared animals of other breeds. Those skilled in the art are familiar with breed standards and/or characteristics. In many embodiments, a particular breed is produced and/or maintained by mating particular identified parent or parents with one another.

Carrier: As used herein, “carrier” or in some cases a “nanoparticle carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components. In some embodiments, a carrier can include a nanoparticle. In some particular embodiments, a carrier can include a nanotube, such as a carbon nanotube, a single-walled nanotube, a chitosan wrapped nanotube, or any combination thereof.

Chloroplast: A type of plastid that contains chlorophyll and can carry out photosynthesis. A chloroplast contains multiple copies of a plant cell plastome.

Chromosome: As used herein, the term “chromosome” refers to a DNA molecule, optionally together with associated proteins and/or other entities, for example as found in the nucleus of eukaryotic cells. Typically, a chromosome carries genes and functions (e.g., origin of replication, etc) that permit it to transmit hereditary information.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc. In some embodiments, a composition may be used to refer to a plant that has been transformed to express an exogenous protein. In some embodiments, a composition may include a nucleic acid material. In some particular embodiments, a composition may include a nucleic acid conjugated to a carrier.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Engineered: In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide. For example, in some embodiments of the present invention, an engineered polynucleotide comprises a regulatory sequence that is found in nature in operative association with a first coding sequence but not in operative association with a second coding sequence, is linked by the hand of man so that it is operatively associated with the second coding sequence. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Excipient: as used herein, refers to a non-therapeutic agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Functional: As used herein, a “functional” biological molecule is a biological molecule in a form in which it exhibits a property and/or activity by which it is characterized.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the fragment.

Gene: As used herein, the term “gene” refers to a DNA sequence in a chromosome that codes for a product (e.g., an RNA product and/or a polypeptide product). In some embodiments, a gene includes coding sequence (i.e., sequence that encodes a particular product); in some embodiments, a gene includes non-coding sequence. In some particular embodiments, a gene may include both coding (e.g., exonic) and non-coding (e.g., intronic) sequences. In some embodiments, a gene may include one or more regulatory elements that, for example, may control or impact one or more aspects of gene expression (e.g., cell-type-specific expression, inducible expression, etc.).

Genome: As used herein, the term “genome” refers to the total genetic information carried by an individual organism or cell, represented by the complete DNA sequences of its chromosomes.

Heterologous: As used herein, “heterologous” with respect to sequence means a sequence that originates from a foreign species, or, if from the same species, is substantially modified from its native form in composition and/or genomic locus by deliberate human intervention.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Host: The term “host” is used herein to refer to a system (e.g., a cell, organism, etc) in which a polypeptide of interest is present. In some embodiments, a host is a system that is susceptible to infection with a particular infectious agent. In some embodiments, a host is a system that expresses a particular polypeptide of interest. In some embodiments, a host system is a plant.

Host cell: as used herein, refers to a cell into which exogenous nucleic acids, for example DNA or RNA (recombinant or otherwise) has been introduced. Persons of skill will understand, upon reading this disclosure, that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous nucleic acid (e.g., a recombinant nucleic acid sequence). In some embodiments, a host cell is a plant cell.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

“Improve,” “increase”, “inhibit” or “reduce”: As used herein, the terms “improve”, “increase”, “inhibit”, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Introduced: “Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

In vitro: The term “in vitro” as used herein refers to events that occur in an artificial environment, e.g., in a test tube or reaction vessel, in cell culture, etc., rather than within a multi-cellular organism.

In vivo: as used herein refers to events that occur within a multi-cellular organism, such as a human and a non-human animal. In the context of cell-based systems, the term may be used to refer to events that occur within a living cell (as opposed to, for example, in vitro systems).

Nanoparticle: As used herein, the term “nanoparticle” refers to a particle having a diameter of less than 1000 nanometers (nm). In some embodiments, a nanoparticle has a diameter of less than 300 nm, as defined by the National Science Foundation. In some embodiments, a nanoparticle has a diameter of less than 100 nm as defined by the National Institutes of Health. In some embodiments, nanoparticles are micelles in that they comprise an enclosed compartment, separated from the bulk solution by a micellar membrane, typically comprised of amphiphilic entities which surround and enclose a space or compartment (e.g., to define a lumen). In some embodiments, a micellar membrane is comprised of at least one polymer, such as for example a biocompatible and/or biodegradable polymer. In some embodiments, a nanoparticle can be a nanotube.

Nanoparticle composition: As used herein, the term “nanoparticle composition” refers to a composition that contains at least one nanoparticle and at least one additional agent or ingredient. In some embodiments, a nanoparticle composition contains a substantially uniform collection of nanoparticles as described herein. In some embodiments, a nanoparticle composition contains a nanoparticle conjugated to another agent (e.g. a drug, agent, nucleic acid material).

Nanoparticle membrane: As used herein, the term “nanoparticle membrane” refers to the boundary or interface between a nanoparticle outer surface and a surrounding environment. In some embodiments, the nanoparticle membrane is a polymer membrane having an outer surface and bounding lumen.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3 -methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5 -propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Nucleic Acid Material: As used herein, “a nucleic acid material” in its broadest sense, refers to any composition comprising a one or more nucleic acid substance, alone or in combination with another component or agent. In some embodiments, a nucleic acid material can include one or more exogenous nucleic acid sequences alone or in combination with one or more endogenous nucleic acid sequences. In some embodiments, a nucleic acid material can be a DNA construct.

Oral: The phrases “oral administration” and “administered orally” as used herein have their art-understood meaning referring to administration by mouth of a compound or composition. In some embodiments, oral administration may refer to feeding a non-human subject.

Operably linked: as used herein, refers to a juxtaposition wherein the components described are in a relationship permitting them to function in their intended manner. A control element “operably linked” to a functional element is associated in such a way that expression and/or activity of a functional element is achieved under conditions compatible with the control element. In some embodiments, “operably linked” control elements are contiguous (e.g., covalently linked) with the coding elements of interest; in some embodiments, control elements act in trans to or otherwise at a from the functional element of interest. In the context of two or more nucleic acid fragments, “operably linked” may refer, for example, to the association of two or more DNA fragments in a DNA construct so that the function of one, e.g. protein-encoding DNA, is controlled by the other, e.g. a promoter.

Pharmaceutical composition: As used herein, the term “pharmaceutical composition” refers to an active agent, formulated together with one or more pharmaceutically acceptable carriers. In some embodiments, an active agent is present in a unit dose amount appropriate for administration in a therapeutic regimen that shows a statistically significant probability of achieving a predetermined therapeutic effect when administered to a subject. In some embodiments, an active agent can be a transformed plant (e.g., a transgenic plant). In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Phenotype: As used herein, the term “phenotype” refers to a trait, or to a class or set of traits displayed by a cell or organism. In some embodiments, a particular phenotype may correlate with a particular allele or genotype. In some embodiments, a phenotype may be discrete; in some embodiments, a phenotype may be continuous.

Plant: includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores. Plants of the present disclosure may include, without limitation, food crops, economic crops, vegetable crops, fruits, flowers, grasses, trees, industrial raw material crops, feed crops or medicine crops. Food crops can include rice, maize, soybean, beans, yams, potato, hulless barley, broad bean, wheat, barley, millet, rye, oat, sorghum, and triticale, etc. Economic crops include, without limitation, oil tea, rape, rapeseed, flax, false flax (Camelina sativa), peanut, oil flax (Linum usitatissimum), mariguana (Cannabis sativa), sunflower, tobacco, cotton, beet, sugarcane, etc. Vegetable crops can include, but are not limited to, radish, Chinese cabbage, tomato, cucumber, hot pepper, carrot, etc. Fruits can include, but are not limited to pear, apple, walnut, cherry, strawberry, jujube or peach; said flowers include flowers for view, for example, orchid, chrysanthemum, carnation, rose, green plants, etc. Grasses and trees include, without limitation, populus, hevea brasiliensis, taxus chinensis, and those for urban greening or those living in deserts and harsh conditions such as drought. Industrial raw material crops include Russian dandelion, guayule, jatropha curcas, etc. Feed crops include, without limitation, the foodstuff for livestock, such as millet, sorghum, oats, wheat, alfalfa, barley, duckweed, clover, grass, corn, hay, straw, silage, sprouted grains, legumes (such as bean sprouts, fresh malt, or spent malt) etc.; Drug crops include, without limitation, ginseng, angelica and ganoderma.

Plastid: A type of membrane-bound organelle found in cells of plants, algae, and other eukaryotic cells that commonly carry one or more of chlorophyll or other pigment(s), fats, proteins, starches, or other compounds.

Plastome: As used herein, a “plastome” refers to the genome of a plastid. Each chloroplast contains multiple copies of the plastome.

Progeny: comprises any subsequent generation of a plant or other living organism.

Polypeptide: As used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Prevent or prevention: as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Promoter: As used herein, “promoter” refers to a DNA regulatory element for initializing transcription. A plant promoter is a promoter capable of initiating transcription in plant cells whether or not its origin is a plant cell, e.g. it is well known that Agrobacterium promoters are functional in plant cells. Thus, plant promoters include promoter DNA obtained from plants, plant viruses and bacteria such as Agrobacterium and Bradyrhizobium bacteria. Examples of promoters under developmental control include promoters that preferentially initiate transcription in certain tissues, such as leaves, roots, or seeds. Such promoters are referred to as “tissue preferred”. Promoters that initiate transcription only in certain tissues are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may affect transcription by inducible promoters include anaerobic conditions, or certain chemicals, or the presence of light. Tissue specific, tissue preferred, cell type specific, and inducible promoters constitute the class of “non-constitutive” promoters. A “constitutive” promoter is a promoter which is active under most conditions. Promoters useful in the present invention are not specifically limited. Those skilled in the art may select suitable promoters according to their knowledge.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Pure: As used herein, an agent or entity is “pure” if it is substantially free of other components. For example, a preparation that contains more than about 90% of a particular agent or entity is typically considered to be a pure preparation. In some embodiments, an agent or entity is at least 91%, at least 92%, at least 93%, at least 94%, at least 95%, at least 96%, at least 97%, at least 98%, or at least 99% pure.

Recombinant: as used herein, is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc).

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Response: As used herein, a response to treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. Techniques for assessing response include, but are not limited to, clinical examination, positron emission tomography, chest X-ray CT scan, MM, ultrasound, endoscopy, laparoscopy, presence or level of tumor markers in a sample obtained from a subject, cytology, and/or histology. The exact response criteria can be selected in any appropriate manner, provided that when comparing groups of tumors and/or patients, the groups to be compared are assessed based on the same or comparable criteria for determining response rate. One of ordinary skill in the art will be able to select appropriate criteria.

Risk: as will be understood from context, “risk” of a disease, disorder, and/or condition refers to a likelihood that a particular individual will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0, 1, 2, 3, 4, 5, 6, 7, 8, 9, 10, or more.

Sample: As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secreations, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not and/or does not comprise a protein or polypeptide (e.g., is not an oligopeptide or peptide). In some embodiments, a small molecule is not and/or does not comprise a polynucleotide (e.g., is not an oligonucleotide). In some embodiments, a small molecule is not and/or does not comprise a polysaccharide; for example, in some embodiments, a small molecule is not a glycoprotein, proteoglycan, glycolipid, etc.). In some embodiments, a small molecule is not a lipid. In some embodiments, a small molecule is a modulating agent (e.g., is an inhibiting agent or an activating agent). In some embodiments, a small molecule is biologically active. In some embodiments, a small molecule is detectable (e.g., comprises at least one detectable moiety). In some embodiments, a small molecule is a therapeutic agent. Those of ordinary skill in the art, reading the present disclosure, will appreciate that certain small molecule compounds described herein may be provided and/or utilized in any of a variety of forms such as, for example, crystal forms, salt forms, protected forms, pro-drug forms, ester forms, isomeric forms (e.g., optical and/or structural isomers), isotopic forms, etc. Those of skill in the art will appreciate that certain small molecule compounds have structures that can exist in one or more stereoisomeric forms. In some embodiments, such a small molecule may be utilized in accordance with the present disclosure in the form of an individual enantiomer, diastereomer or geometric isomer, or may be in the form of a mixture of stereoisomers; in some embodiments, such a small molecule may be utilized in accordance with the present disclosure in a racemic mixture form. Those of skill in the art will appreciate that certain small molecule compounds have structures that can exist in one or more tautomeric forms. In some embodiments, such a small molecule may be utilized in accordance with the present disclosure in the form of an individual tautomer, or in a form that interconverts between tautomeric forms. Those of skill in the art will appreciate that certain small molecule compounds have structures that permit isotopic substitution (e.g., ²H or ³H for H; ¹¹C, ¹³C or ¹⁴ C for 12C; , ¹³N or ¹⁵N for 14N; ¹⁷O or ¹⁸O for 16O; ³⁶Cl for XXC; ¹⁸F for XXF; 131I for XXXI; etc). In some embodiments, such a small molecule may be utilized in accordance with the present disclosure in one or more isotopically modified forms, or mixtures thereof. In some embodiments, reference to a particular small molecule compound may relate to a specific form of that compound. In some embodiments, a particular small molecule compound may be provided and/or utilized in a salt form (e.g., in an acid-addition or base-addition salt form, depending on the compound); in some such embodiments, the salt form may be a pharmaceutically acceptable salt form. In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source. Thus, in some embodiments, for example, a preparation of a single stereoisomer of a small molecule compound may be considered to be a different form of the compound than a racemic mixture of the compound; a particular salt of a small molecule compound may be considered to be a different form from another salt form of the compound; a preparation that contains only a form of the compound that contains one conformational isomer ((Z) or (E)) of a double bond may be considered to be a different form of the compound from one that contains the other conformational isomer ((E) or (Z)) of the double bond; a preparation in which one or more atoms is a different isotope than is present in a reference preparation may be considered to be a different form; etc.

Stable: The term “stable,” when applied to compositions herein, means that the compositions maintain one or more aspects of their physical structure and/or activity over a period of time under a designated set of conditions. In some embodiments, the period of time is at least about one hour; in some embodiments the period of time is about 5 hours, about 10 hours, about one (1) day, about one (1) week, about two (2) weeks, about one (1) month, about two (2) months, about three (3) months, about four (4) months, about five (5) months, about six (6) months, about eight (8) months, about ten (10) months, about twelve (12) months, about twenty-four (24) months, about thirty-six (36) months, or longer. In some embodiments, the period of time is within the range of about one (1) day to about twenty-four (24) months, about two (2) weeks to about twelve (12) months, about two (2) months to about five (5) months, etc. In some embodiments, the designated conditions are ambient conditions (e.g., at room temperature and ambient pressure). In some embodiments, the designated conditions are physiologic conditions (e.g., in vivo or at about 37° C. for example in serum or in phosphate buffered saline). In some embodiments, the designated conditions are under cold storage (e.g., at or below about 4° C., −20° C., or −70° C.). In some embodiments, the designated conditions are in the dark.

Subject: As used herein, the term “subject” or “test subject” refers to any organism to which a provided compound or composition is administered in accordance with the present disclosure e.g., for experimental, diagnostic, prophylactic, and/or therapeutic purposes. Typical subjects include animals (e.g., mammals such as mice, rats, rabbits, chickens, goats, cows, cattle, non-human primates, and humans; insects; worms; etc.) and plants. In some embodiments, a non-human animal may be a monogastric animal, for example, swine, poultry, or horses. In some embodiments, a non-human animal may be a ruminant animal, for example, cattle, sheep, and/or goats. In some embodiments, a subject may be suffering from, and/or susceptible to a disease, disorder, and/or condition.

Substantial identity: as used herein refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., Basic local alignment search tool, J. Mol. Biol., 215(3): 403-410, 1990; Altschul et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with and/or displays one or more symptoms of a disease, disorder, and/or condition.

Susceptible to: An individual who is “susceptible to” a disease, disorder, and/or condition is one who has a higher risk of developing the disease, disorder, and/or condition than does a member of the general public. In some embodiments, an individual is an animal of a particular species or breed of animal (e.g., a cow, chicken, goat, or sheep) that has a higher risk of developing a certain disease or disorder. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Symptoms are reduced: According to the present invention, “symptoms are reduced” when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom

Systemic: The phrases “systemic administration,” “administered systemically,” “peripheral administration,” and “administered peripherally” as used herein have their art-understood meaning referring to administration of a compound or composition such that it enters the recipient's system.

Therapeutic agent: As used herein, the phrase “therapeutic agent” refers to an agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Transformation: as used herein, refers to any process by which exogenous DNA is introduced into a host cell. Transformation may occur under natural or artificial conditions using various methods well known in the art. Transformation may rely on any known method for the insertion of foreign nucleic acid sequences into a prokaryotic or eukaryotic host cell. In some embodiments, a particular transformation methodology is selected based on the host cell being transformed and may include, but is not limited to, viral infection, electroporation, mating, lipofection, or using a chemical and/or nano- or micro-particle aid. In some embodiments, a “transformed” cell is stably transformed in that the inserted DNA is capable of replication either as an autonomously replicating plasmid or as part of the host chromosome (e.g., in a nucleus or chloroplast). In some embodiments, a transformed cell transiently expresses introduced nucleic acid for limited periods of time.

Transgenic plant: “Transgenic plant” as used herein, refers to a plant which comprises within its genome a heterologous polynucleotide. Preferably, the heterologous polynucleotide is stably integrated within the genome such that the polynucleotide is passed on to successive generations. The heterologous polynucleotide may be integrated into the genome alone or as part of a recombinant DNA construct.

Trait: As used herein, the term “trait” refers to a detectable attribute of an individual. Typically, expression of a particular trait may be fully or partially influenced by an individual's genetic constitution. In some embodiments, a trait is characteristic of a particular individual, line, breed or crossbreed, for example in that it can be relied upon (individually or as part of a set) to distinguish that individual, line, breed, or crossbreed from others.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Vaccination or Vaccine: As used herein, the term “vaccination” refers to the administration of a composition intended to generate an immune response, for example to a disease-causing agent. For the purposes of the present invention, vaccination can be administered before, during, and/or after exposure to a disease-causing agent, and in certain embodiments, before, during, and/or shortly after exposure to the agent. In some embodiments, vaccination includes multiple administrations, appropriately spaced in time, of a vaccinating composition. As used herein, the term “vaccine” refers to any composition intended to generate an immune response. In some embodiments a vaccine includes a transgene organism, engineered to express and antigen.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

Vector: as used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

### DETAILED DESCRIPTION OF CERTAIN EMBODIMENTS

The present description encompasses, inter alia, immunogenic compositions such as plant-based vaccine compositions including modified plants, or portions thereof, and methods of administering said compositions to animals, such as ruminant livestock. Methods of modifying plants to express an exogenous nucleic acid sequence, for example, encoding an antigen of interest, are also disclosed. In some embodiments, methods of introducing one or more exogenous nucleic acid sequence(s) into a host plant cell, including e.g., via transformation. In some embodiments, transformation of a plant cell includes transformation of the exogenous nucleic acid sequence into a plastome (e.g. a chloroplast genome) of the host plant cell. In some embodiments, an exogenous nucleic acid sequence is passed on to progeny. Methods of producing plant-based vaccines using certain plant host species (e.g., sorghum and millet) are described herein and methods for administering the same.

In cattle feedlot operations, macrolide antibiotics are widely deployed to control the negative effects of F. necrophorum, with tylosin being most effective (Brown et al., 1973; Brown et al., 1975; Tadepalli et al., 2009). However, since this class of antibiotic is also used as a human therapeutic to control a wide variety of ailments resulting from bacterial infection and inflammation, the beef industry is seeking alternative treatments.

Studies of the F. necrophorum vaccine Fusogard have shown some prevention of foot rot and decreased probability of liver abscesses in background fed cattle, but the protective effects seemed overwhelmed by high-grain diets (Checkley et al., 2005). Efforts to improve vaccine efficacy by Sun et al. (2009) led to the identification of two highly immunoprotective subregions of ltkA, named PL1 and PL4. In spite of this progress, the cumbersome and laborious nature of subcutaneous injection (e.g. need for trained medical personal, logistical challenge when large numbers of animals are involved), has limited the practical number of vaccinations and, as such, has limited the effectiveness of this treatment. Additionally, other challenges are associated with conventional vaccines including cost, stability of a vaccine during a storage period, and the need for intensive processing and storage. These factors make it particularly challenging to use conventional vaccines and administration methods to vaccinate large numbers of livestock animals in a feedlot.

The ease and relative safety of using a plant-based vaccine as described herein can make the production of such crops attractive for controlling, or in some cases, preventing disease. Edible vaccines pose an alternative path to immunity, particularly in maladies where the site of invasion are the mucosal surfaces of the gastrointestinal tract. Plant-based edible vaccines have been shown to stimulate mucosal and systemic humoral responses and their bioencapsulation by the cell wall protects from premature digestion in the stomach (Lakshmi et al., 2013). In addition, they can be administered through simple pasture/trough feeding and are overall, a low-cost option (Kwon and Daniell, 2015).

Transgenic plants conduct all relevant post-translational processes (folding, glycosylation, etc.) for proper three dimensional assembly of immunogenic antigens. Research into the use of transgenic crops for oral immunization of animals has resulted in a number of limited successes, including immunization of mice fed with transgenic tomato expressing enterovirus protein (Chen et al., 2006) induction of immunoprotective responses in sheep fed with maize expressing rabies virus glycoprotein (Loza-Rubio et al., (2012), and oral vaccination of ruminants (cattle and sheep) fed with liver fluke antigen produced by transgenic lettuce (Wesolowska et al., 2018).

Some success of using plant-based edible vaccines in veterinary studies has also been shown (for review, see Jacob et al., 2013; and Takeyama et al., 2015), and the first effective plant-based oral vaccine for ruminants was reported by Loza-Rubio et al. (2012) and more recent successes were shown by Wesolowska et al. (2018).

Among the most important challenges to developing an efficacious edible vaccine in plants is sufficiently high ectopic expression levels of antigenic material (Rybicky, 2009; Rojas-Anaya et al., 2009). Chloroplast transformation is an attractive approach for overcoming expression shortfalls in that each plant cell has 10,000 copies of chloroplast genome from which to express constructs (Shahid and Daniell, 2016), and transplastomic expression studies have shown to produce as much as >70% of total soluble protein (Ruhlman et al., 2010, see also McBride et al., 1995). Other advantages include maternal inheritance that decreases transgene dispersal (Heifetz, 2000), polycistronic expression per transformation event (Hanson et al., 2013), and reduced gene silencing resulting from homologous recombination (see Adem et al., 2017 and references therein).

While most of the successes in chloroplast transformation have been reported in the Nicotiana genus (Rigano et al., 2012), in addition to other dicotyledonous crops like soybean, lettuce, and alfalfa (Cardi et al., 2010, Wei et al., 2011), similar successes are rarer in cereals of the family Poaceae, as monocotyledonous plants, appear to be obdurate with current transgenic methods.

Herein, several methods are described for introducing exogenous genes into host plant genomes, such as cereal species of sorghum and millet. One method is targeting the chloroplast genome of the host plant for e.g., site-specific integration.

Thus, in some embodiments, the current approach to synthesizing immunogenic compositions (e.g., plant-based compositions), capable of expressing certain antigens (e.g. leukotoxin or fragments thereof) described here will be directed towards integration of exogenous nucleic acid material into the host species' plastid genome (e.g., chloroplast genome), inter alia, via homologous recombination, resulting in a putative vaccine suitable for oral administration to ruminant livestock.

Certain challenges remain in the development of plant-based vaccines including species-specific challenges, particularly when using chloroplast transformation. For example, stability, storage, formulation, and/or dosing and administration, as well as public opinions of genetically-modified plants have and do pose challenges in the development and use of plant-based vaccines. Additionally, certain cereal crops, including sorghum and millet, have proved resistant to many methods of transformation, including Agrobacterium-mediated nuclear transformation, and successfully targeting and integrating one or more transgenes into a chloroplast genome for expression of an exogenous antigen sequence has not been demonstrated in these species.

In general, successful chloroplast transformation relies on, inter alia, detailed plastomic sequence information to identify regions of the chloroplast genome suitable for homologous recombination and safe transgene incorporation, for example. However, in addition to the raw sequence information, considerable work must be done in order to determine optimal, or even viable, sites for the integration of an exogenous nucleic acid (e.g., a transgene of interest). Other challenges associated with developing and/or administering plant-based vaccines are addressed herein and include development of target-specific nucleic acid constructs that can integrate into a particular location within the chloroplast genome of, for example, sorghum, millet, and triticeae species, and lead to expression of an antigen (e.g. leukotoxin or fragments thereof).

Another feature of the methods and compositions encompassed by the present disclosure is the ability to monitor the degree and nature of successful transformation and/or expression of exogenous nucleic acid sequence(s) in plants. For example, one approach to monitoring expression of exogenous genes in plants is to co-express one or more markers (“selection markers”), for example, those which emit fluorescence under appropriate conditions. Such markers include green fluorescent proteins (GFP) which has been identified in the jellyfish Aequorea victoria (Ormo et al., 1996), along with A. victoria mutants that result in cyan fluorescent proteins (Goedhard et al., 2012) and yellow fluorescent proteins (YFP, Nagai et al., 2002), as well as red fluorescent protein identified in the mushroom anemone discosoma species (DsRED, Bevis et al., 2002). In some embodiments, the present disclosure provides for the use of one or more of such proteins to confirm incorporation and/or expression of transgenic material.

In order to properly express foreign proteins, it is necessary to equip the genes coding for these proteins with appropriate DNA signatures to facilitate normal cellular processing of genetic material. In general, three major classes of DNA signatures are necessary for foreign protein expression; two at the 5′ end of the coding regions, and one at the 3′ end. At the 5′ end are: the promoter—a DNA signature that serves as an RNA binding site, and the 5′ untranslated region (also called a leader sequence) which has assists the newly produced RNA in binding to the ribosome. At the 3′ end, a transcription terminator sequence is necessary to disengage the transcriptional complex and mark the end of transcription.

Taken together, in some embodiments, a nucleic acid material is designed to deliver exogenous nucleic acid sequence(s) encoding e.g., an antigen of interest, to a plastome (e.g., a chloroplast genome) for homologous recombination integration and may comprise 1) DNA signatures that complement the host specie's chloroplast, 2) one or more transgenes encoding one or more antigens of interest, and 3) one or more genetic markers, along with 4) the genetic machinery to properly to translate and express the transgenes. In some embodiments, such machinery may be exogenously supplied and/or under the control of a non-native control mechanism, in whole or in part.

In some embodiments, such machinery may be endogenous to the plant and/or plant organelle, in whole or in part.

### Plant Species

In accordance with various embodiments, any of a wide variety of plant species used in accordance with methods encompassed by the present disclosure, for example, to integrate and express an exogenous nucleic acid (e.g., encoding an antigen of interest). A plant species or host species of the present disclosure may include, without limitation, whole plants, mature plants, plant organs, plant tissues, seeds and plant cells and progeny of same. Plant cells may include, without limitation, one or more of cells from seeds, seedlings, suspension cultures, embryos, meristematic regions, callus tissue, leaves, roots, shoots, gametophytes, sporophytes, pollen, and microspores. Plants of the present disclosure may include, without limitation, food crops, economic crops, vegetable crops, legumes, fruits, flowers, grasses, trees, industrial raw material crops, feed crops or medicine crops.

Food crops, such as cereal crops, can include rice, maize, soybean, beans, yams, potato, hulless barley, broad bean, wheat, barley, garlic, millet, rye, oat, triticale, sudangrass, soybeans, and sorghum. Economic crops can include, without limitation, oil tea, canola, grapeseed, flax, false flax (Camelina sativa), peanut, oil flax (Linum usitatissimum), marijuana (Cannabis sativa), sunflower, tobacco, cotton, beet, sugarcane.

Vegetable crops can include, but are not limited to, radish, Chinese cabbage, tomato, cucumber, onion, corn, leafy greens (e.g., spinach, kale, collard, chard, and lettuce), mustard, sweet potato, cabbage, celery, beet, beets, radish, turnip, hot pepper, carrot, asparagus, broccoli, cabbage, cauliflower, eggplant, pepper, and potato.

Fruits can include, without limitation, pear, apple, walnut, cherry, strawberry, jujube or peach. Flowers include flowers for view, for example, orchid, chrysanthemum, carnation, rose, and green plants.

Grasses and trees can include, without limitation, populus, hevea brasiliensis, taxus chinensis, and those for urban greening or those living in deserts and harsh conditions such as drought.

Feed crops can include any plant used to feed domesticated livestock, such as cattle, rabbits, sheep, horses, chickens and pigs, for example, for livestock grazing, or the foodstuff for livestock. Examples include, but are not limited to, millet, sorghum, oats, wheat, alfalfa, barley, duckweed, clover, grass, corn, hay, straw, silage, sprouted grains, legumes (such as bean sprouts, fresh malt, or spent malt).

Example drug crops include, but are not limited to, ginseng, angelica and ganoderma.

In some embodiments, a plant species of the present disclosure may be a cross of any of plant species of sub-species. For example, in some embodiments, a cereal species can include a cross of two sorghum species. In some embodiments, a sorghum species includes sorghum sudangrass, resultant from a cross of (Sorghum bicolor ((L.) Moench)×(Sorghum×drummondii) (Nees ex. Steud.)).

### Nucleic Acid Material

Nucleic acid material of the present disclosure may include nucleic acids alone or in combination with one or more other agents or compositions. In some embodiments, a nucleic acid material can also refer to a DNA construct. In accordance with various embodiments, components of a nucleic acid material can include, without limitation, one or more targeting sequence(s), selection sequence(s), exogenous DNA sequence(s), enhancer sequence(s), promoter sequence(s), and termination sequence(s).

In some embodiments, a nucleic acid material is or comprises a RNA oligonucleotide, a DNA oligonucleotide, a plasmid, or any combination thereof. A DNA oligonucleotide can be a single-stranded DNA oligonucleotide, a double-stranded DNA oligonucleotide. In some embodiments, a DNA oligonucleotide can be from any DNA source, including, but not limited to, genomic DNA, plasmid DNA, phage DNA, cDNA, synthetic DNA sequence, or any other appropriate source of DNA. In some embodiments, an RNA oligonucleotide may comprise one or more of mRNA, snRNA, siRNA, or miRNA oligonucleotide.

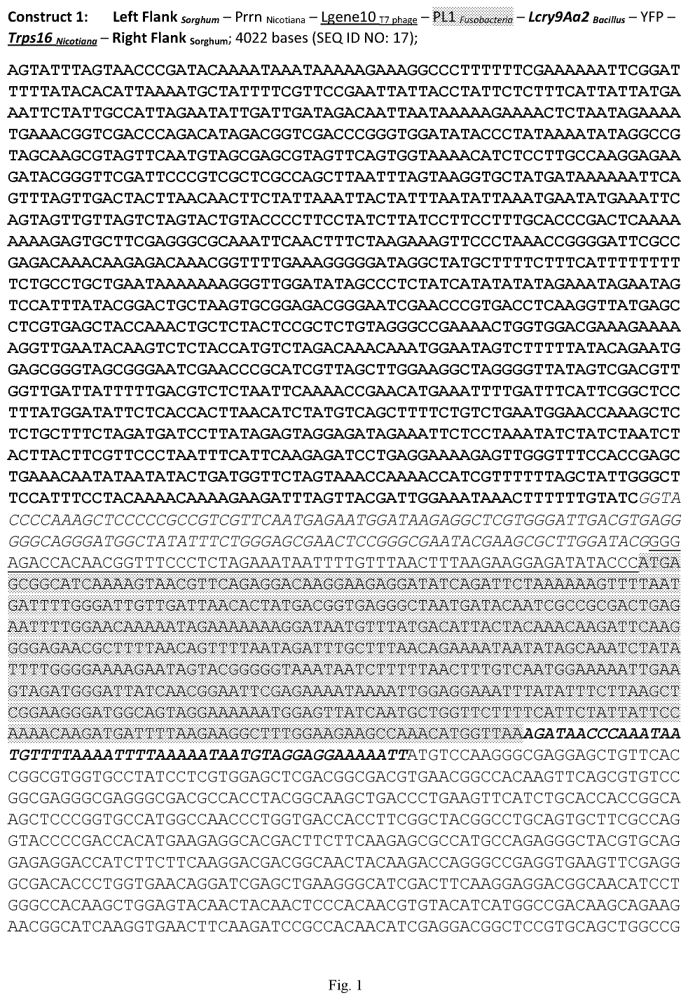

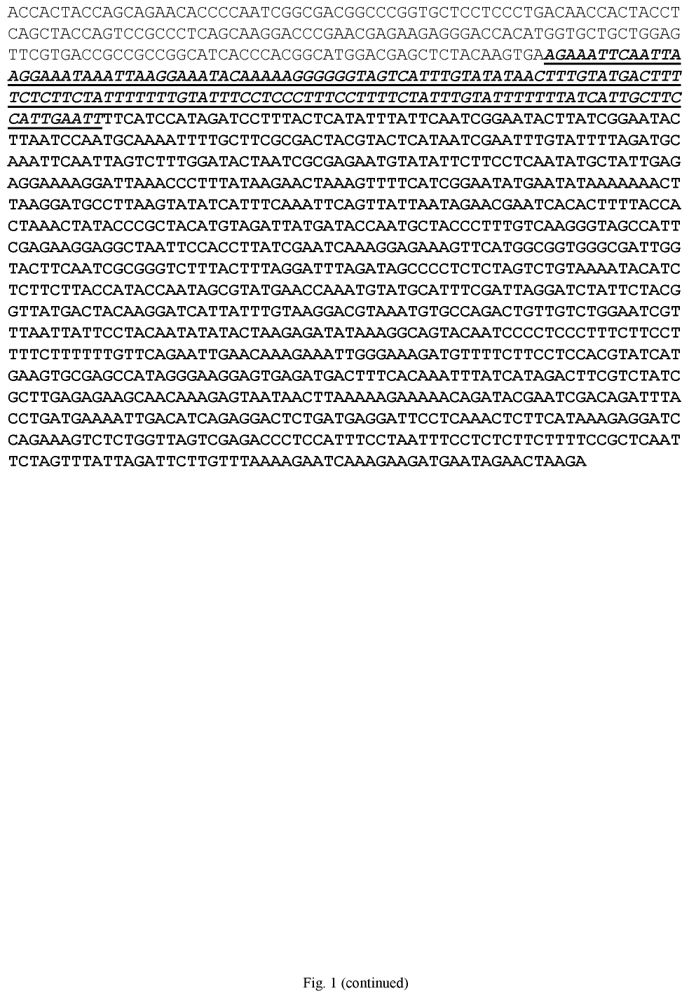

In some embodiments, a nucleic acid material may include a DNA construct that is at least 70%, 75%, 80%, 85%, 90%, 95%, or 100% identical to a DNA sequence including elements as described above, and shown, e.g., in constructs 1-4 in FIGS. 1-4 (SEQ ID NOs: 17-20). A DNA construct of the present disclosure can include a DNA construct that includes any combination of the components shown in constructs 1-4 in FIGS. 1-4.

### Exogenous Nucleic Acid Sequence

An exogenous nucleic acid sequence, as the term is used herein, refers to any nucleic acid that is non-native to an organism or host cell (i.e. is not normally expressed in a particular organism, also referred to as a “transgene”).

In some embodiments, an exogenous nucleic acid sequence may be or comprise a nucleic acid sequence encoding more than one transgene of interest. In some embodiments, an exogenous nucleic acid sequence may encode a polypeptide of interest, for example, monoclonal antibodies, fragment antigen binding (Fab) fragments, cytokines, receptors, antigens, human vaccines, animal vaccines, and plant polypeptides. In some embodiments, a transgene is an immunogenic portion of an antigen of interest.

Antigens

In some embodiments, an exogenous nucleic acid sequence may encode a particular antigen or antigenic fragment. In some embodiments, an exogenous nucleic acid sequence encoding an antigen or antigenic fragment, when introduced into a plant cell, may function as a vaccine when consumed by a subject, such as a human or animal. In some embodiments, an exogenous nucleic acid sequence of interest may include, without limitation, a sequence encoding a virus (e.g., a pathogenic virus, for example, including a virulence factor) or portion such as a fragment or variant thereof, a bacteria (e.g., a pathogenic bacteria) or portion such as a fragment or variant thereof, or a fungi (e.g., a pathogenic fungi) or portion such as a fragment or variant thereof, or protozoa (e.g., a pathogenic protozoa) or portion such as a fragment or variant thereof. In some embodiments, an antigen may be or comprise an immunogenic portion or fragment of a full length protein or peptide provided by or otherwise associated with a pathogenic virus (including a virulence factor), a pathogenic bacteria, pathogenic fungi, and/or a pathogenic protozoa.

Examples of pathogenic viruses may include, without limitation, single stranded RNA viruses (with and without envelope), double stranded RNA viruses, and single and double stranded DNA viruses such as (but not limited to) tobacco mosaic virus, tobacco rattle virus, pea enation mosaic virus, barley stripe mosaic virus, potato viruses X and Y, carnation latent virus, beet yellows virus, maize chlorotic virus, tobacco necrosis virus, turnip yellow mosaic virus, tomato bushy stunt virus, southern bean mosaic virus, barley yellow dwarf virus, tomato spotted wilt virus, lettuce necrotic yellows virus, wound tumor virus, maize streak virus, and cauliflower mosaic virus.

In some embodiments, an antigen is or comprises a bacterium or portion such as a fragment or variant thereof, for example, a virulence factor produced from a bacterium, or a fragment or variant thereof. In some embodiments, a virulence factor could be produced from bacterium that commonly infects ruminant livestock, or another non-human animal. In some embodiments, a bacterium can include, without limitation, Fusobacterium necrophorum (including e.g. one of its subspecies F. necrophorum subsp. necrophorum and F. necrophorum subsp. Funduliforme), Mannheimia (Pasteurella) haemolytica, Actinobacillus actinomycetemcomitans, P. haemolytica, A. actinomycetemcomitans, Examples of bacterial pathogens include bacteria from the following genera and species: Chlamydia (e.g., Chlamydia pneumoniae, Chlamydia psittaci, Chlamydia trachomatis), Legionella (e.g.,Legionella pneumophila), Listeria (e.g., Listeria monocytogenes), Rickettsia (e.g., R. australis, R. rickettsii, R. akari, R. conorii, R. sibirica, R. japonica, R. africae, R. typhi, R. prowazekii), Actinobacter (e.g., Actinobacter baumannii), Bordetella (e.g., Bordetella pertussis), Bacillus (e.g., Bacillus anthracis, Bacillus cereus), Bacteroides (e.g., Bacteroides fragilis), Bartonella (e.g., Bartonella henselae), Borrelia (e.g., Borrelia burgdorferi), Brucella (e.g., Brucella abortus, Brucella canis, Brucella melitensis, Brucella suis), Campylobacter (e.g., Campylobacter jejuni), Clostridium (e.g., Clostridium botulinum, Clostridium difficile, Clostridium perfringens, Clostridium tetani), Corynebacterium (e.g., Corynebacterium diphtherias, Corynebacterium amycolatum), Enterococcus (e.g., Enterococcus faecalis, Enterococcus faecium), Escherichia (e.g., Escherichia coli), Francisella (e.g., Francisella tularensis), Haemophilus (e.g., Haemophilus influenzae), Helicobacter (e.g., Helicobacter pylori), Klebsiella (e.g., Klebsiella pneumoniae), Leptospira (e.g., Leptospira interrogans), Mycobacteria (e.g., Mycobacterium leprae, Mycobacterium tuberculosis), Mycoplasma (e.g., Mycoplasma pneumoniae), Neisseria (e.g., Neisseria gonorrhoeae, Neisseria meningitidis), Pseudomonas (e.g., Pseudomonas aeruginosa), Salmonella (e.g., Salmonella typhi, Salmonella typhimurium, Salmonella enterica), Shigella (e.g., Shigella dysenteriae, Shigella sonnei), Staphylococcus (e.g., Staphylococcus aureus, Staphylococcus epidermidis, Staphylococcus saprophyticus), Streptococcus (e.g., Streptococcus agalactiae, Streptococcus pneumoniae, Streptococcus pyogenes), Treponoma (e.g., Treponoma pallidum), Vibrio (e.g., Vibrio cholerae, Vibrio vulnificus), and Yersinia (e.g., Yersinia pestis).

In some embodiments, a virulence factor can include generally, without limitation, an endotoxin and/or an exotoxin. In some embodiments, a virulence factor can include, without limitation, Cholera toxin, Tetanus toxin, Botulinum toxin, Diphtheria toxin, Streptolysin, Pneumolysin, Alpha-toxin, Alpha-toxin, Phospholipase C, Beta-toxin, Streptococcal mitogenic exotoxin, Streptococcal pyrogenic toxins, Leukotoxin A, hemagglutinin, hemolysin, hyaluronidase, protease, coagulase, lipases, deoxyribonucleases and enterotoxins, M protein, lipoteichoic acid, hyaluronic acid capsule, destructive enzymes (including streptokinase, streptodornase, and hyaluronidase), streptolysin, alin A, internalin B, lysteriolysin O, actA, and Cytolethal distending toxin.

Examples of protozoal pathogens include the following organisms: Cryptosporidium parvum, Entamoeba (e.g., Entamoeba histolytica), Giardia (e.g., Giardia lambila), Leishmania (e.g., Leishmania donovani), Plasmodium spp. (e.g., Plasmodium falciparum, Plasmodium vivax, Plasmodium ovale, Plasmodium malariae), Toxoplasma (e.g., Toxoplasma gondii), Trichomonas (e.g., Trichomonas vaginalis), and Trypanosoma (e.g., Trypanosoma brucei, Trypanosoma cruzi). Libraries for other protozoa can also be produced and used according to methods described herein.

Examples of fungal pathogens include the following: Aspergillus, Candida (e.g., Candida albicans), Coccidiodes (e.g., Coccidiodes immitis), Cryptococcus (e.g., Cryptococcus neoformans), Histoplasma (e.g., Histoplasma capsulatum), and Pneumocystis (e.g., Pneumocystis carinii).

In some embodiments, a transformed plant cell, for example functioning as or producing a plant-based vaccine, may be used to treat and/or prevent a common disease in ruminant livestock including, but not limited to Acetonaemia, acidosis, Acorn Poisoning, Anaplasmosis, Anthrax, Blackleg, Bloat, Bluetongue, Botulism, Bovine Anaemia, Bovine Babesiosis, Bovine Respiratory Disease Complex (BRDC), Bovine spongiform encephalopathy (B SE), Bovine Trichomoniasis, Bracken Poisoning, BRSV (Bovine Respiratory Syncytial Virus), Brucellosis, BVD (Bovine Viral Diarrhea), Calf Diphtheria, Calf Pneumonia, Calf Scour, Clostridial Disease, Coccidiosis, Cold Cow Syndrome, Copper Poisoning, Cryptosporidiosis, Cystic ovaries, Digital Dermatitis, Displaced Abomasum, Epizootic Hemorrhagic Disease, Fatty Liver, Fog Fever, Foot and Mouth, Foot Rot, foot thrush, Gut Worms, Haemophilus Somnus, Hypermagnesaemia, IBR (Infectious Bovine Rhinotracheitis), Infectious Bovin Rhinotracheitis (IBR), Johnes, Joint Ill, Lead Poisoning, Leptospirosis, Lice, Listeriosis, liver abscess, Liver Fluke, Mange, Mastitis, Molybdenum Toxicity, Necrotic Enteritis, Neosporosis, New Forest Eye, Nitrate poisoning, Pasteurella Haemolytica Pasteurella Multocida , Peri-Weaning Diarhheoa, Photosensitisation, PI3 (Parainfluenza Type 3), Pruritus/Pyrexia/Haemorrhagic Syndrome, pseudocowpox, Rabies, Ragwort Poisoning, Rain Scald, Repeat Breeding Syndrome, Retained Fetal Membranes, Rift Valley Fever, Ringowrm, Rotaviral Diarrhoea, Rumen Acidosis, rumenitis, Samonella, Schmallenberg, Selenium Deficiency, Sole Ulcer, Summer Mastitis, Tetanus, Thrombosis, Traumatic Reticuliti, Trypanosomosis, Tuberculosis (TB), Ulcerative Mammillitis, Vibriosis, and Wooden Tongue.

In some embodiments, an antigen may include an immunogenic fragment, variant, or truncation of a sequence encoding any one of the above-identified antigens and/or antigens from any of the above identified organisms. In some embodiments, truncations of leukotoxin A (e.g., as identified in Sun et al. 2009) can be used to elicit immunoprotective effects in organisms challenged with Fusobacterium infection. In some embodiments, an exogenous nucleic acid sequence encodes a peptide comprising a sequence that is at least 70%, 75%, 80%, 85%, 90%, 95% or 100% identical to a leukotoxin A (ltkA) protein represented by GenBank: DQ672338.1, or a fragment or variant thereof. In some embodiments an immunogenic fragment of ltkA can include a sequence encoding a region of ltkA selected from the group consisting of PL1 (GenBank: DQ672338.1 1-501), PL4 (DQ672338.1 5637-6606, and a combination of P1 and PL4 (as shown in the DNA constructs 1-4 in FIGS. 1-4), or any fragment or variant thereof. In some embodiments an immunogenic fragment of ltkA can include a sequence that is at least 70%, 75%, 80%, 85%, 90%, 95% or 100% identical to a sequence encoding at least one region of ltkA selected from the group consisting of PL1 (DQ672338.1 1-498), PL2 (DQ672338.1 946-1911), PL3(DQ672338.1 3950-6052), PL4 (DQ672338.1 5637-6606), PL5 (DQ672338.1 9226-9721) (e.g., as shown in the DNA constructs 1-4 in FIGS. 1-4), or any fragment or variant thereof.

In some embodiments, an exogenous nucleic acid sequence may comprise a sequence that encodes an immunogenic fragment, variant, or truncation of a full native antigen sequence. In some embodiments, an exogenous nucleic acid sequence may include a sequence that encodes an immunogenic fragment variant, or truncation of a native antigen sequence that is at least 70%, 75%, 80%, 85%, 90%, 95% or 100% identical to a native antigen sequence, or a fragment thereof.

In some embodiments, an exogenous nucleic acid sequence can include a sequence of one or more different transgenes, encoding e.g., one or more proteins, e.g., one or more antigens. In some embodiments, an exogenous nucleic acid sequence can include a sequence of one or more immunogenic fragments from one antigen. In some embodiments, an exogenous nucleic acid sequence can include a sequence of one or more immunogenic fragments from multiple antigens.

In addition to the exogenous nucleic acid sequence, a nucleic acid material may include one or more control elements operably linked to an exogenous nucleic acid in a manner that permits and/or enhances its transcription, translation and/or expression in a cell transformed with a nucleic acid material. Expression control sequences can include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A number of expression control sequences, including promoters that are native, constitutive, inducible and/or tissue-specific, are known in the art and may be included in a vector described herein.

### Promoters

In addition to an exogenous nucleic acid sequence encoding a transgene of interest, a nucleic acid material (e.g., a DNA construct), may include one or more promotors in proximity (upstream) to the exogenous nucleic acid sequence, to initiate transcription of a protein encoded by the exogenous nucleic acid sequence (e.g., an antigen). A promoter may be “operably linked,” e.g., associated with one or more DNA fragments (e.g., an exogenous nucleic acid) in a nucleic acid material so that the function of one or more DNA fragments, e.g. protein-encoding DNA, are controlled by the promoter.

In some embodiments, a promoter is naturally occurring in the genome of a host cell, also referred to as an endogenous promoter. In some embodiments, an endogenous promoter may be used to control a gene that is not normally associated with that promoter (e.g., a transgene). In some embodiments, a promoter sequence may have at least 70%, 80%, 85%, 90%, 95%, 98% or 99% identity to a native or endogenous promoter. In some embodiments, a promoter is a non-natural or exogenous promoter.

In some embodiments, a nucleic acid material may include a constitutive promoter. In some embodiments, a constitutive promoter can comprise a native or non-native promoter that is operably linked to an exogenous nucleic acid sequence, for example, encoding a transgene of interest. In some embodiments, a constitutive promotor is part of a constitutive expression construct and may include a recombinant expression vector described herein.

In some embodiments, a nucleic acid material may include a regulated promoter. In some embodiments, a regulated promoter can comprise a native or non-native promoter that is operably linked to an exogenous nucleic acid sequence encoding a transgene of interest. In some embodiments, a regulated promotor is part of a regulatable expression construct and may include a recombinant expression vector described herein.

In some embodiments, a promoter can be a plant promoter, capable of initiating transcription in a host plant. In some embodiments, promoters can include any promoter DNA obtained from plants, plant viruses and/or bacteria such as Agrobacterium and Bradyrhizobium bacteria. Examples of promoters under developmental control can include promoters that preferentially initiate transcription in certain tissues, such as leaves, roots, or seeds, i.e., “tissue preferred” promoters. In some embodiments, a promoter can be a “tissue specific”, i.e. promoters that initiate transcription only in certain tissues are referred to as “tissue specific”. In some embodiments, a promoter can be a “cell type” specific promoter, i.e., a promoter that primarily drives expression in certain cell types in one or more organs, for example, vascular cells in roots or leaves.

Example promoters include, without limitation, common CMV, ElF, VAV, TCRvbeta, MCSV, PGK, PpsbA, Prrn, Prna, psaA, PrbcL, CaMV35S, rbcS,Patpl and PatpB, or an A3 or RS324 promoter. Additional types of promoter may be used, and may depend, for example, on the species of the host plant. In some embodiments, a plant promoter can be derived from any known plant including for example, food crops, economic crops, vegetable crops, legumes, fruits, flowers, grasses, trees, industrial raw material crops, feed crops or medicine crops.

In some embodiments, where, e.g., a nucleic acid material such as a DNA construct includes more than one exogenous nucleic acid sequence, a promoter can be operably linked to each exogenous nucleic acid sequence. In some embodiments where a nucleic acid material includes multiple promoters, each of the promoters may be the same or different promoters.

### Targeting Sequences

A nucleic acid material may include one or more targeting sequences, e.g., in order to be integrated into a particular location within the host genome. In some embodiments, more than one targeting sequence may be used, for example, a first and a second targeting sequence. Targeting sequences, in some embodiments, are nucleic acid sequences that are complementary, e.g., at least 80, 85, 90, 95, 98 or 99% complementary, e.g., fully complementary, to a target sequence on a nucleic acid of interest in, for example, a plant e.g., a sequence that is complementary to an endogenous nucleic acid sequence to the host cell (e.g., a sequence that is adjacent to a desired integration point).

In some embodiments, a first and/or second targeting sequence are designed to be complementary to regions of a host genome that flank (e.g., are adjacent to) a target endogenous nucleic acid sequence and/or target integration site for a transgene. In some embodiments, the host is a plant cell and the endogenous nucleic acid sequence is a sequence that is an endogenous sequence within a host genome (e.g., a plastome). In some embodiments, a plant cell is from any of the plants described above. In some embodiments, targeting sequences are complementary to sequences within a nuclear genome. In some embodiments, targeting sequences are complementary to sequences within a chloroplast genome. In some embodiments, a chloroplast genome can be the chloroplast genome of sorghum plant species (as represented by sorghum (Sorghum bicolor (L.) Moench, Genbank: NC_008602.1), the chloroplast of millet (e.g. “Broomcorn Millet” Panicum miliaceum L., GenBank: KU343177.1; “Little millet” Panicum sumatrense, NCBI accession number KX756177; “Pearl millet” Cenchrus americanus/Pennisetum americanum/P. glaucum, NCBI accession number KJ490012; “Foxtail millet” Setaria italic, NCBI accession number NC_022850)or the chloroplast genome of any Triticeae species (e.g., as described in Middleton et al. 2013 PLoS One 9.3 (2014): e85761; e.g., Triticum aestivum, Genbank: FN645450.1, KC912694.1, or NC 002762.1).

In some embodiments, targeting sequences may flank a target region (e.g., a site of desired transgene integration) or endogenous region that is between two genes within a nuclear genome.

In some embodiments, targeting sequences may flank a target region or endogenous region that is between two genes within a chloroplast genome. For example, in some embodiments the two genes may include a first and second gene within the genome of a chloroplast and/or nuclear genome. In some embodiments, the first chloroplast gene may be selected from the following genes trnI, trnA, trnM, trnG, rrn16, rps12/7, tscA, psac, trnV, trnA, rbcL, accD, rp132, trnL, 3′rps12/7, trnV, petA, psbJ, Trn16/V, 16srrnA, trnfM, trnG, atpB, rbcL, trN, trnR, Ycf3, trnS, Rps7, ndhB, trnY, GUA, trnD, GUC, trnG, UCC, trnM, trnT, and/or CAU. In some embodiments, the second chloroplast gene may be selected from the following genes trnl, trnA, trnM, trnG, rrn16, rps12/7, tscA, psac, trnV, trnA, rbcL, accD, rp132, trnL, 3′rps12/7, trnV, petA, psbJ, Trn16/V, 16srrnA, trnfM, trnG, atpB, rbcL, trN, trnR, Ycf3, trnS, Rps7, ndhB, trnY, GUA, trnD, GUC, trnG, UCC, trnM, trnT, and/or CAU.

In some embodiments, a first and second targeting sequence are directed to a target sequence located between chromosomal coordinates of two genes selected from trnI-trnA, trnM-trnG, rrn16-rps12/7, tscA-psac, trnV-trnA, rbcL-accD, rp132-trnL, 3′rps12/7-trnV, petA-psbJ, Trn16/V-16srrnA, trnfM-trnG, atpB-rbcL, trN-trnR, Ycf3-trnS, Rps7-ndhB, trnY-GUA-trnD-GUC, trnG-UCC-trnM-CAU, and trnT-trnL.

A targeting sequence may be described by the position (i.e., coordinates) of the complementary region it targets within a host chloroplast genome (i.e., sorghum chloroplast genome, millet chloroplast genome, etc). One of skill in the art will appreciate that the same targeting sequence may be described by different coordinates dependent upon the particular version of the sequenced genome obtained. For many plant species, their chloroplast genome has been sequenced by different groups, and there exists several versions that vary to some degree, be it from species variation or even local variations within a particular species due to known rearrangements of genetic material over time. In this case, a targeting sequence may be described based on its sequence or the sequence it aims to target, rather than the particular position (i.e., coordinates) within the host genome that it targets. It is contemplated that one of skill in the art could ascertain the coordinates within a particular version of the sequenced chloroplast genome based on the unique targeting sequence.

In some embodiments, targeting sequences of a nucleic acid material as disclosed herein, can include sequences that have at least 70%, 80%, 85%, 90%, 95%, 98% or 99% identity to SEQ ID NOs: 1, 8, 15, 16, and 23).

In some embodiments, a first and second targeting sequence correspond to bases 47318 through 48218 and 48219 through 49116, respectively of the millet chloroplast genome (Genbank accession KU343177). In some embodiments, a first and second targeting sequence correspond to 16408 through 16845, and 16846 through 17960, respectively of the millet chloroplast genome (Genbank accession KU343177). In some embodiments, a first and second targeting sequence correspond to bases 46391 through 47746 and 47747 through 49115 for the millet chloroplast genome (Genbank accession KU343177).

In some embodiments, a first and second targeting sequence correspond to bases 14048 through 14793 and 14794 through 15561 of the sorghum chloroplast genome (Genbank accession NC_008602). In some embodiments, a first and second targeting sequence correspond to bases 13151 through 14490 and 14491 through 15560 of the sorghum chloroplast genome (Genbank accession NC_008602).

### Enhancer Elements

In various aspects of the disclosure, a nucleic material of the present disclosure may include one or more enhancer sequences, for example, to increase transcription of an exogenous nucleic acid. For example, in some embodiments, one or more enhancer sequences can be included at the 5′ untranslated region (also called a leader sequence) which may assist the newly produced RNA in binding to the ribosome.

In some embodiments, an enhancer sequence can include one or more enhancer sequences selected from: ggagg, rrn 5′UTR, T7genel0 5′ UTR, LrbcL 5′UTR, LatpB 5′UTR, Tobacco mosaic virus omega prime 5′UTR (GenBank: KM507060.1), Lcry9Aa2 5′UTR, atpl 5′UTR, psbA 5′UTR, cry2a, rrnB, rps16, petD, psbA, pabA, and any combination or variant thereof.

In some embodiments, a nucleic acid material of the present disclosure may include one or more termination sequences. In some embodiments, a termination sequence can include tobacco Trps16 (GenBank accession MF580999), TpsbA, TrbcL, TrpL32, and TpetD.

In some embodiments, the one or more enhancers included in a nucleic acid material can include any one of the enhancer sequences identified in SEQ ID NOs: 3, 5, 9, 11, and 13 (and as shown in the DNA constructs of FIGS. 1-4).

### Selection Sequences

In accordance with various embodiments, nucleic acid materials, e.g., DNA constructs as described herein, can include one or more selection sequences. In some embodiments, selection sequences may be used to provide an efficient system for identification of those cells that have been successfully transformed and transiently and/or stably express an exogenous nucleic acid sequence, for example, after receiving and integrating a DNA construct into their genomes. In some embodiments, a selection sequence may provide (e.g., facilitate or allow the expression of) one or more selection markers which confer resistance to a selection agent, such as an antibiotic or herbicide. Then, for example, potentially transformed cells may be exposed to the selection agent, and the population of surviving cells will be those cells where, generally, the resistance-conferring gene is integrated and expressed at sufficient levels to permit cell survival. In some embodiments, cells may be tested further to confirm stable integration of the exogenous DNA. Commonly used selection sequences may encode genes conferring resistance to antibiotics such as kanamycin and paromomycin (nptII), hygromycin B (aph IV) and gentamycin (aac3 and aacC4), spectinomycin and streptomycin resistance gene (aadA) or resistance to herbicides such as glufosinate (bar or pat) and glyphosate (aroA or EPSPS). In some embodiments, a gene conferring resistance to antibiotics is a 16S rRNA gene, e.g., a 16SrRNA gene with one or more mutations. In some embodiments, resistance to antibiotics is passive resistance. In some embodiments, resistance to antibiotics is “binding-type” resistance. Examples of such selection sequences and/or selection agents are illustrated in U.S. Pat. Nos. 5,550,318; 5,633,435; 5,780,708 and 6,118,047, all of which are incorporated herein by reference. In some embodiments, an antibiotic selection sequence can include a nucleic acid sequence encoding a spectinomycin resistance gene, a gentamycin resistance gene, a streptomycin resistance gene, a Kanamycin resistance gene, a neomycin resistance gene, a Beta lactam resistance gene, or any combination thereof.

In some embodiments, a selection sequence may also provide an ability to visually identify transformants (e.g., by encoding an observable moiety), for example, a nucleic acid sequence encoding a colored or fluorescent protein such as a luciferase or green fluorescent protein (GFP), yellow fluorescent protein (YFP), red fluorescent protein (RFP), cyan fluorescent protein (CFP), a His tag, GUS uidA lacz, or a gene expressing a beta glucuronidase or uidA gene (GUS) for which various chromogenic substrates are known, or any combination thereof.

In some embodiments, a selection sequence can be or include one or more of the selection sequences encoding yellow fluorescent protein (YFP, GenBank: GQ221700.1 or SEQ ID NO: 6), red fluorescent protein (DsRED, GenBank: KY426960.1 or SEQ ID NO: 12), or cyan fluorescent protein (CFP, GenBank: HQ993060.1 or SEQ ID NO: 14) (as shown in the constructs of FIGS. 1-4).

### Vectors

In some embodiments, a vector is used for expression and/or integration of a nucleic acid material (i.e., DNA construct) in a host cell. In some embodiments, a vector has a copy number that is more than 25, 50, 75, 100, 150, 200, or 250 copies per cell. In accordance with various embodiments, useful vectors for polypeptide expression in plants include viral vectors or plasmids. Examples, without limitation include lentiviral vectors, adenoviral vectors, adeno-associated viral vectors (AAVs), pET vectors (Novagen), Gateway® pDEST vectors (Invitrogen), pGEX vectors (Amersham Biosciences), pPRO vectors (BD Biosciences), pBAD vectors (Invitrogen), pLEX vectors (Invitrogen), pMAL™ vectors (New England BioLabs), pGEMEX vectors (Promega), and pQE vectors (Qiagen). Vector systems for producing phage libraries are known and include Novagen T7Select° vectors, pMX vector plasmid (Invitrogen's GeneArt Gene Synthesis), and New England Biolabs Ph.D.™ Peptide Display Cloning System. In some embodiments, a vector may be or comprise a plant-specific vector. In some embodiments, a plant-specific vector can be or include Ti plasmid of Agrobacterium tumefaciens, tobacco mosaic virus (TMV), potato virus X, cauliflower mosaic virus (CaMV) 35S promoter, Bean yellow dwarf virus, geminiviruses, Wheat dwarf virus (WDV), Wheat streak mosaic virus (WSMV), Barley stripe mosaic virus (BSMV), Cabbage leaf curl virus (CaLCuV), Tobacco rattle virus (TRV), and cowpea mosaic virus.

### Methods for Introducing Nucleic Acid Material

Various methods may be used for introducing (i.e., transforming, transducing and/or transfecting) a nucleic acid material into a plant cell. The introduction of a nucleic acid material into a plant may occur via any suitable technique, including, but not limited to, direct DNA uptake, chemical treatment, electroporation, microinjection, cell fusion, infection, vector mediated DNA transfer, bombardment (e.g., gene gun), nanoparticle-guided biomolecule delivery, liposome, protoplast, callus, silicon carbide fiber, and pollen tube transformation, or Agrobacterium mediated transformation. Methods including some form of bombardment can include, without limitation, methods known in the art, including using the biolistic device PDSI000/He (Bio-Rad) as described in U.S. Patent Publication No.: US20060117412A1, and Daniell 1997 (Nature Biotech, (16):345-348).

In some embodiments, it may be useful to introduce recombinant DNA randomly, i.e. at a non-specific location, in the genome of a target plant. In some embodiments, after introduction (i.e. transformation) of a nucleic acid material into a plant cell, a portion of the exogenous nucleic acid sequence in the nucleic acid material is removed, such as a selection marker. In some embodiments, it may be useful to target insertion of the nucleic acid material in order to achieve site-specific integration, for example to replace an existing gene in the genome, to use an existing promoter in the plant genome, or to insert a recombinant polynucleotide at a predetermined site known to be active for gene expression. Several site specific recombination systems exist which are known to function in plants include cre-lox as disclosed in U.S. Pat. No. 4,959,317 and FLP-FRT as disclosed in U.S. Pat. No. 5,527,695, both incorporated herein by reference.

In some embodiments, an exogenous nucleic acid sequence is introduced (e.g., transformed, transduced, and/or transfected) into a plastome. In some embodiments, an exogenous nucleic acid sequence is introduced into a chloroplast genome of a plant cell. In some embodiments, an exogenous nucleic acid sequence is introduced into a nuclear genome of a plant cell. In some embodiments, introducing an exogenous nucleic acid sequence is performed such that the plant cell is stably, that is, permanently transformed with the exogenous nucleic acid sequence (e.g., through site-specific homologous recombination), including the progeny thereof. In some embodiments, a stably transformed exogenous nucleic acid material is capable of autonomous expression of a nucleotide coding region in a plant cell to produce at least one polypeptide (e.g., antigen). In such instances, introducing an exogenous nucleic acid sequence into a plant cell is performed so that the plant cell may transiently express an exogenous nucleic acid sequence (i.e., an antigen).

In some embodiments, transformation methods encompassed by this disclosure may be practiced in vitro and/or in a controlled environment. Recipient cell targets can include, but are not limited to, meristem cells, callus, immature embryos and gametic cells such as microspores, pollen, sperm and egg cells. In accordance with various embodiments, it is contemplated that any cell from which a fertile plant may be regenerated is useful as a recipient cell. Callus may be initiated from tissue sources including, but not limited to, immature embryos, seedling apical meristems, microspores and the like. Cells capable of proliferating as callus are also recipient cells for genetic transformation. Practical transformation methods and materials for making transgenic plants of this invention, for example various media and recipient target cells, transformation of immature embryo cells and subsequent regeneration of fertile transformed plants are disclosed in U.S. Pat. Nos. 6,194,636 and 6,232,526, which are incorporated herein by reference.

In some embodiments, plants comprising one or more nucleic acid materials in accordance with the present disclosure may be self-pollinated to provide homozygous transformed plants. In other embodiments, pollen obtained from a plant comprising one or more nucleic acid materials is crossed to seed-grown plants of agronomically important lines. In still other embodiments, pollen from plants comprising one or more nucleic acid materials may be used to pollinate naturally occurring plants. A transformed plant of the present invention comprising an exogenous nucleic acid sequence encoding, e.g., an antigen, may be cultivated using methods known to one skilled in the art.

### Viral Vectors

As is described herein, various methods of delivering nucleic acid material to a host cell may be used. In some embodiments, an exogenous nucleic acid sequence as described herein can be introduced into a plant cell in a viral vector.

Vector Design

In some embodiments, a viral vector can be derived from any known plant-based or plant-compatible viral vector. A viral vector may be chosen based on a number of factors, for example, the plant species being transformed, size of the exogenous nucleic acid and location targeted within the host genome. Viral DNA of a viral vector for modifying plants is, for example, designed and constructed to optimize infectivity, movement throughout the plant host cell, and high multiplication.

In some embodiments, an exogenous nucleic acid sequence as described herein can be cloned into a number of types of vectors. For example, a nucleic acid can be cloned into a plasmid, a phagemid, a phage derivative, an animal virus, a plant virus, or a cosmid.

In some embodiments, a virus can include, for example, Ti plasmid of Agrobacterium tumefaciens, tobacco mosaic virus (TMV), potato virus X, cauliflower mosaic virus (CaMV) 35S promoter, Bean yellow dwarf virus, geminiviruses, Wheat dwarf virus (WDV), Wheat streak mosaic virus (WSMV), Barley stripe mosaic virus (BSMV), Cabbage leaf curl virus (CaLCuV), Tobacco rattle virus (TRV), Tomato golden mosaic virus (TGMV), Alfalfa Mosaic Virus (A1MV), ilarviruses, cucumoviruses such as Cucumber Green Mottle Mosaic virus (CGMMV), Tobacco Etch Virus (TEV), Cowpea Mosaic virus (CMV), and viruses from the brome mosaic virus group such as Brome Mosaic virus (BMV), broad bean mottle virus, cowpea chlorotic mottle virus, Rice Necrosis virus (RNV), Cassaya latent virus (CLV) and maize streak virus (MSV). Alternative vectors can include expression vectors, replication vectors, probe generation vectors, and sequencing vectors, and non-plant derived viral vectors.

In some embodiments, vectors may have one or more transcription termination regions. A transcription termination region is a sequence that controls formation of the 3′ end of the transcript, e.g., polyadenylation sequences and self-cleaving ribozymes. Termination signals for expression in other organisms are well known in the literature. Sequences for accurate splicing of the transcript may also be included. Examples are introns and transposons.

Viral vector design and technology is well known in the art as described in Sambrook et al, (Molecular Cloning: A Laboratory Manual, 2001), and in other virology and molecular biology manuals.

Viral Transduction

Viruses are highly efficient at nucleic acid delivery to specific cell types, while often avoiding detection by the infected host immune system. These features make certain viruses attractive candidates as vehicles for introduction of nucleic acid material into target cells (e.g., plant cells). A number of viral based systems have been developed for gene transfer into mammalian and plant cells. In general, a suitable vector comprises an origin of replication functional in at least one organism, a promoter sequence, convenient restriction endonuclease sites, and one or more selectable markers. A viral vector described herein can be in DNA or RNA form.

In some embodiments, a viral vector can be used to deliver exogenous nucleic acid sequences of various sizes to a host cell (e.g., a plant cell). In some embodiments, a viral vector can accommodate an exogenous nucleic acid sequence that is greater than 50, 100, 200, 400, 500, 1000 nucleotides in length.

In some embodiments, an exogenous nucleic acid sequence can be cloned into a viral vector and then introduced into a host cell (e.g., a plant cell). In some embodiments, viral vectors can be introduced into a plant host cell using bombardment (e.g., gene gun), Agrobacterium mediated transformation, or any other method encompassed by the present disclosure.

Any of a variety of methods for facilitating infection of a target plant can be applied to cell(s) of the plant according to any technique known to those skilled in the art. For example, in some embodiments, suitable techniques include, but are not limited to, hand inoculations such as abrasive inoculations (leaf abrasion, abrasion in a buffer solution), mechanized spray inoculations, vacuum infiltration, particle bombardment and/or electroporation.

In some embodiments, a viral vector can be delivered to a plant at different growth stages such as seedling stage, leaf stage, flowering, seed formation and maturation stages through roots, cotyledons, leaves, seed coat, seeds, pods, stem inoculations, etc. In some embodiments, a viral vector can be applied at one or more locations of a host plant. For example, a viral vector can be applied on leaves and roots either simultaneously or successively. In some embodiments, a viral vector can be applied at the same location (e.g., on a given leaf) more than once at successive intervals. The time intervals can depend on the experimental conditions and the target gene to be silenced. Two types of vectors (e.g. local and systemic) capable of introducing two different genes can be mixed and applied at a given location or more than one location. Once applied, samples can be collected and screened for virus infection.

In some embodiments, a viral vector may be designed and constructed for systemic infection. In some embodiments, a viral vector can also be engineered in a manner that initiation of target gene silencing also initiates destruction and elimination of the vector from plant (approximately 15-20 days after inoculation). In some embodiments, a viral vector may be designed and constructed for localized infection, e.g., if a leaf is infected, the infection does not spread beyond said leaf.

### Nanoparticles and Nanotubes

In some embodiments, nucleic acid materials as described herein may be delivered to and/or transformed into a host cell (e.g., a plant cell) via a nanoparticle.

In some embodiments, a nanoparticle is a particle having a diameter of less than 1000 nanometers (nm), less than 300 nm, or less than 100 nm. In some embodiments, nanoparticles are micelles in that they comprise an enclosed compartment, separated from the bulk solution by a micellar membrane, typically comprised of amphiphilic entities which surround and enclose a space or compartment (e.g., to define a lumen). In some embodiments, a micellar membrane is comprised of at least one polymer, such as for example a biocompatible and/or biodegradable polymer.

In some embodiments, a nanoparticle may have or comprise a nanoparticle membrane or boundary or interface between a nanoparticle outer surface and a surrounding environment. In some embodiments, the nanoparticle membrane is a polymer membrane having an outer surface and bounding lumen.

In some embodiments, a nanoparticle is conjugated to the nucleic acid material. In some embodiments, a nanoparticle is conjugated to an exogenous nucleic acid sequence to be delivered to a host cell (e.g., plant cell).

In some embodiments, a nanoparticle can be a nanotube. It has been demonstrated that certain nanotubes have the ability to traverse rigid cell walls in plant cells, including the double lipid bilayers of chloroplasts. In some embodiments, a nanoparticle, such as a nanotube, is sized and dimensioned so that the nanoparticle can penetrate the cell membrane and, for example, a chloroplast envelope in a plant cell. In some embodiments, nanoparticle size and surface charge are selected based on the where an exogenous nucleic acid is integrated in a plant cell (e.g., using the lipid exchange envelope penetration (LEEP) model described in Kwak, Seon-Yeong, et al. (2019 Nature nanotechnology (14.5): 447)). In some embodiments, a nanotube is a carbon nanotube. In some embodiments, a nanotube is a single-walled nanotube or a single-walled carbon nanotube (SWCNT). Methods of conjugating an exogenous nucleic acid sequence can be any known method including, but not limited to, those described in Kwak, Seon-Yeong, et al. (2019 Nature nanotechnology (14.5): 447). Conjugating a nucleic acid material to a nanoparticle (e.g., SWCNT) can include incubation of the nanoparticle with the nucleic acid material (e.g., in a dialysis cartridge).

In some embodiments, nucleic acid materials may be delivered to a particular organelle within a plant host genome. In accordance with various embodiments, an organelle may be any organelle within a plant host cell, including a nucleus or chloroplast. In some embodiments, a nanotube may be modified to promote delivery to a particular organelle and/or to promote efficient delivery. In some embodiments, a nanotube or nanoparticle may be covalently modified. In some embodiments, a nanotube or nanoparticle may be non-covalently modified. In some embodiments, a nanotube may be a chitosan-wrapped nanotube and/or a chitosan-wrapped single-walled nanotube (CS-SWNT). In some embodiments, a nanoparticle (e.g., a nanotube) may be PEGylated. In some embodiments, a nanotube may be non-covalently bonded to a 5,000 Mw PEG. In some embodiments, a nanoparticle (e.g., a nanotube) may be modified such that the modifications protect the exogenous nucleic acid from nuclease degradation. In some embodiments, a modified nanoparticle (e.g., a nanotube) has a radius of less than 200 nm, less than 150 nm, less than 100 nm, or less than 50 nm.

In some embodiments, a nanoparticle is designed and constructed (e.g., using chitosan) so that the nucleic acid material is conjugated to a nanoparticle in one location within a plant cell (e.g., within the plant cytosol) and be release from the nanoparticle in another location (e.g., within the chloroplast stroma). In some embodiments, a nanoparticle is designed and constructed so that the nanoparticle is released from the nucleic acid material upon exposure to an environment that has a pH of greater than 6.0, greater than 6.5, greater than 7.0, greater than 7.5, or greater than 8.0.

In some embodiments, a nanoparticle conjugated to a nucleic acid material is delivered to a plant cell using localized infiltration. In some embodiments, a solution containing a nanoparticle conjugated to a nucleic acid material is infused into a part or parts of a plant.

In some embodiments, a solution is infused in an amount of about 1-1,000 μl, 20-1,500 μl, 30-1,000 μl, 40-750 μl, 50-500 μl, 100 μl-10 ml. In some embodiments, a solution is infused in an amount of at least 1 μl, 10 μl, 100 μl, 1000 μl, 2 ml, 3 ml, 4 ml, 5 ml, 6 ml, 7 ml, 8 ml, 9 ml, or 10 ml. In some embodiments, the amount of nucleic acid material that is delivered to a plant cell is about 1 ng, 5 ng, 10 ng, 20ng, 50 ng, or greater. In some embodiments, the amount of nucleic acid material that is delivered to a plant cell is about 1 μg, 5 μg, 10 μg, 20 μg, 50 μg, or greater. In some embodiments, the ratio of nanoparticle to nucleic acid material is at least 1:1, 3:1, or 6:1 (w/w).

In some embodiments, a nanoparticle conjugated to a nucleic acid material is delivered to a plant cell with or without the use of biolistic force. In some embodiments, nanoparticle conjugated to a nucleic acid material is delivered to a plant cell using methods that include, e.g., abaxial surface leaf infusion through a needleless syringe and/or stem injection through a needled syringe.

### Expression of Exogenous Nucleic Acid Sequence(s)

In some embodiments, the present disclosure includes a plant that has been transformed such that the plastome (e.g., chloroplast genome) of the plant or plant cell has been stably, that is, permanently transformed in accordance with methods of the invention (e.g., through site-specific homologous recombination), including the progeny thereof. In some embodiments, a nucleic acid material comprises one or more cloning or expression vectors; for instance, a vaccine comprising one or more of the compositions or transformed plants as described herein may comprise a plurality of expression vectors each capable of autonomous expression of a nucleotide coding region in a plant cell to produce at least one immunogenic polypeptide. In such instances, a transformed plant may transiently express an exogenous nucleic acid sequence (i.e., an antigen). In some embodiments, a transformed plant contains an exogenous nucleic acid sequence where the expression of the sequence (i.e., an antigen) is driven by a promoter that is constitutively expressed. In some embodiments, a transformed plant contains an exogenous nucleic acid sequence where the expression of the sequence (i.e., an antigen) is driven by a promoter that is differentially expressed, e.g., in the absence or presence of light.

In some embodiments, expression of exogenous nucleic acid material is detectable 1 hour after transformation/inoculation of the host species. In some embodiments, expression of exogenous nucleic acid material remains detectable for at least 1, 2, 3, 4, 5, 6, 7, 14, or 21 days after transformation/inoculation of the host species. In some embodiments, expression of exogenous nucleic acid material remains detectable for at least 1, 2, 3, 4, 5, 6, or 12 months after transformation/inoculation of the host species.

In some embodiments, detecting transformation of a plant cell can be determined when the expression of an exogenous nucleic acid sequence is greater than the expression in a control cell (i.e., a non-transformed cell). In some embodiments, detecting transformation of a plant cell can be determined when the expression of an exogenous nucleic acid sequence is greater than at least 0%, 10%, 20%, 30%, 40%, 50%, 60%, 70%, 80%, or 90% or greater than the expression in a control cell (i.e., a non-transformed cell).

Methods of measuring expression may include, without limitation, southern blot analysis using probes that can detect a particular nucleotide sequence, or amplification of a transgene by PCR. Methods of measuring/detecting expression of an exogenous protein (e.g., an antigen) produced by a transformed plant as encompassed by the present disclosure include, without limitation, ELISA (enzyme-linked immunosorbent assay), Western blotting, competition assay, and spot-blot. Means of detection may be or include, for instance, chemiluminesce, fluorescence, or colorimetric detection. One suitable method for measuring binding of the antigen, using a known antibody, is the Luminex xMAP system, where peptides are conjugated to a dye-containing microsphere. In some embodiments, other systems are used to assay a plurality of markers, for example, profiling may be performed using any of the following systems: antigen microarrays, bead microarrays, nanobarcodes particle technology, arrayed proteins from cDNA expression libraries, protein in situ array, protein arrays of living transformants, universal protein array, lab-on-a-chip microfluidics, and peptides on pins. Another type of clinical assay is a chemiluminescent assay to detect antigen-antibody binding.

### Production

In accordance with various embodiments, any of a variety of methods for growing/producing transformed plants, and formulating said transformed plants into immunogenic compositions (e.g. plant-based vaccines) may be used. As used herein, the term “plant-based vaccine” or “plant-based vaccine composition” includes compositions comprising one or more parts of a plant or one or more components produced in a plant (e.g., an exogenous nucleic acid sequence). Method of production and/or formulation may depend e.g., on the species of the subject the immunogenic composition is being administered to, the type of plant, or the antigen of interest to be expressed in the transformed plant.

Various methods of growing and propagating transformed plants may include any systems or procedures used in farming and agriculture, and may depend on the plant species used in a particular application. In some embodiments, seeds of a transformed plant can be harvested from fertile transformed plants, and can be used to grow progeny generations of transformed plants. In some embodiments, a selection sequence is used to select the plants that have been transformed with the exogenous nucleic acid sequence. In addition to direct transformation of a plant with a nucleic acid material, transformed plants can be prepared by crossing a first transformed plant with a second non-transformed plant. For example, an exogenous nucleic acid sequence encoding an antigen protein can be introduced into first plant line that is amenable to transformation to produce a transgenic plant, which can be crossed with a second plant line to introduce the exogenous nucleic acid into the second plant line.

In some embodiments, a transformed plant expressing an exogenous nucleic acid sequence encoding an antigen of interest (or fragment thereof) is grown to a certain confluence and/or maturity, and then subsequently harvested. In some embodiments, a transformed plant is cut and harvested wet (e.g., containing about 65% moisture). In some embodiments, harvested plant material is treated and/or preserved, e.g., by sun-drying (to cure the plant material). In some embodiments, the harvested plant material is processed (e.g., by dehydration and e.g. further baled). In some embodiments, the harvested plant material is baled and used as dry (e.g., sun-cured) feed for livestock animals.

In some embodiments, a transformed plant is harvested as hay (e.g., air dried 85-90% dry matter). In some embodiments, a transformed plant is harvested as hay is ground through a screen (e.g., a 2-3″ screen). In some embodiments, harvested hay is mixed into a ration to be feed to a non-human animal, e.g., to be 35-45% of the total roughage in the ration.

In some embodiments, when the harvested plant material is processed, e.g., by dehydration, the dried material is further processed, e.g., compressed into pellet form or into a larger block so that it can be fed to livestock animals. The pellets can be administered as supplements, e.g., by a trained professional. In some embodiments, the plant material in compressed, block form, can be placed in a living area of one or more livestock animals so that they can access the block and ingest the plant material by licking the block throughout the day (e.g., have free access to the plant material).

In some embodiments, harvested plant material is processed into silage (crop ensiled). In some embodiments, the harvested plant material is ensiled without drying and the harvested, wet (e.g., containing about 65% moisture) plant material may be fed to livestock animals e.g., daily, every other day, weekly, monthly, or intermittently. In some embodiments, harvested plant material is not ensiled before it is fed to a livestock animal e.g., daily, every other day, weekly, monthly, or intermittently. In some embodiments, the transformed plants are harvested and then directly fed to a livestock animal (e.g., without further processing, e.g., a “green chop”).

In some embodiments, a plant cell producing an antigen of interest (i.e., has been transformed with an exogenous nucleic acid sequence), can be administered to livestock animals by allowing the livestock animal to graze on the live plant cell line producing an antigen. As such, delivery to the animal via grazing is constant, i.e., throughout the day, several times per day, at regular or irregular intervals as grazing of the live plant occurs.

In some embodiments, a transformed plant cell line can be used to grow and expand the plant population expressing a particular antigen, so that it can be harvested and the antigen can be purified from the transformed plant cells, and further processed into a different form, e.g., in the form of a conventional vaccine. In some embodiments, an antigen purified from transformed plant cells can be a fragment of the antigen, such as an immunogenic fragment. In some embodiments, an antigen purified from transformed plant cells can be concentrated to a particular concentration and purity of antigen, depending, for example, on the use of the composition.

In some embodiments, a transformed plant is cultivated to produce a particular protein antigen of interest and can be compared with a control plant. As used herein a “control plant” means a plant that does not contain the exogenous nucleic acid sequence encoding a particular protein antigen of interest or a “non-transformed” plant. A control plant may be used to identify and select a transformed plant that is producing (e.g., expressing) a particular antigen of interest. In some embodiments, a suitable control plant can be a non-transformed plant of the parental line used to generate a transformed plant, i.e. devoid of the exogenous nucleic acid sequence encoding a particular antigen of interest. A suitable control plant may, in some embodiments, be a progeny of a transformed plant line that does not contain an exogenous nucleic acid encoding a particular antigen of interest, known as a negative segregant. Cultivated transformed plants can be harvested and quantified in order to prepare a specific concentration of antigen for an immunogenic composition (e.g., plant-base vaccine i.e. dosage) to be provided to a non-human animal for treatment.

### Immunogenic Compositions

In some embodiments, one or more plants (e.g., a mixture of plants) may be formulated into an immunogenic composition (e.g., a plant-based vaccine) and administered to a subject. By way of a further non-limiting example, specified amounts of a transformed plant (e.g., transgenic plant) can be diluted with a non-transformed plant, for example, to achieve a particular ratio of transformed plant mass to non-transformed plant mass to achieve, inter alia, a desired concentration (or concentration range) of an antigen in the immunogenic composition. In some embodiments, a desired concentration will depend on any of several factors, for example, the timing of use of an immunogenic composition (i.e., whether used prophylactically or for therapeutic treatment), the particular subject (e.g., species, age, size), the progression of the disease or infection being treated, and also the particular dosing regimen desired.

In some embodiments, immunogenic compositions (e.g., plant-base vaccines) may include a delivery system for use in administering a provided immunogenic composition to a subject (e.g., a ruminant animal). In some embodiments a delivery system may comprise a material and/or coating that will resist degradation due to gastric and enteric environments. In some embodiments, a delivery system may include, but is not limited to, a liposome, a proteasome, cochleates, virus-like particles, immune-stimulating complexes, microparticles and nanoparticles (e.g., nanotubes).

In some embodiments, immunogenic compositions may include a transformed plant produced using a system and/or method described herein and an application-appropriate carrier or excipient.

Formulations of immunogenic compositions described herein may be prepared by any method known or hereafter developed in the art. In general, such preparatory methods include the step of bringing a transformed plant into association with a diluent (e.g., a non-transformed plant), a carrier, and/or one or more other accessory ingredients, and then, if necessary and/or desirable, shaping and/or packaging the product into a desired single- or multi-dose unit (e.g., into a pellet or block).

An immunogenic composition in accordance with the present disclosure may be prepared, packaged, and/or sold in bulk, as a single unit dose, and/or as a plurality of single unit doses. As used herein, a “unit dose” is discrete amount of a composition comprising a predetermined amount of at least one plant-based product produced using a system and/or method described herein.

Relative amounts of transformed plant produced using a system and/or method described herein, a carrier, and/or any additional ingredients in a immunogenic composition can vary, depending upon the subject to be treated (e.g., species of non-human animal, age, size), target cells, diseases or disorders, and may also further depend upon the route by which the composition is to be administered.

### Pharmaceutical Compositions

According to some embodiments, an immunogenic composition can include an antigen purified from transformed plant cells that is concentrated to a particular concentration and purity. A purified and/or concentrated antigen may be combined with an additional component e.g., a pharmaceutically effective carrier or excipient into a pharmaceutical composition (e.g., a vaccine).

Pharmaceutical compositions may comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by the United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition.

Pharmaceutical compositions may be formulated such that they are suitable for administration to a human and/or non-human animal subject. In some embodiments, a pharmaceutical composition is substantially free of either endotoxins or exotoxins. Endotoxins include pyrogens, such as lipopolysaccharide (LPS) molecules. A pharmaceutical composition may also be substantially free of inactive protein fragments. In some embodiments, a pharmaceutical composition has lower levels of pyrogens than industrial water, tap water, or distilled water. Other components of a pharmaceutical composition may be purified using methods known in the art, such as ion-exchange chromatography, ultrafiltration, or distillation. In other embodiments, the pyrogens may be inactivated or destroyed prior to administration to a subject. Raw materials for a pharmaceutical composition, such as water, buffers, salts and other chemicals may also be screened and depyrogenated. A pharmaceutical composition may be sterile, and each lot of the pharmaceutical composition may be tested for sterility. Thus, in certain embodiments the endotoxin levels in the a pharmaceutical composition fall below the levels set by the USFDA, for example 0.2 endotoxin (EU)/kg of product for an intrathecal injectable composition; 5 EU/kg of product for a non-intrathecal injectable composition, and 0.25-0.5 EU/mL for sterile water. It is preferred that a pharmaceutical composition has low or no toxicity, within a reasonable risk-benefit ratio.

The formulations suitable for introduction of a pharmaceutical composition vary according to route of administration. Formulations suitable for parenteral administration, such as, for example, by intraarticular (in the joints), intravenous, intramuscular, intradermal, intraperitoneal, intranasal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampoules and vials.

Injection solutions and suspensions can be prepared from sterile powders, granules, and tablets of the kind previously described.

Formulations suitable for oral administration of a pharmaceutical composition can include (a) liquid solutions, such as an effective amount of the polypeptides or packaged nucleic acids suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, tragacanth, microcrystalline cellulose, acacia, gelatin, colloidal silicon dioxide, croscarmellose sodium, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art. In some embodiments, a pharmaceutical composition can be encapsulated, e.g., in liposomes, or in a formulation that provides for slow release of the active ingredient.

A pharmaceutical composition can be made into aerosol formulations (e.g., they can be “nebulized”) to be administered via inhalation. Aerosol formulations can be placed into pressurized acceptable propellants, such as dichlorodifluoromethane, propane, nitrogen, and the like.

Suitable formulations for vaginal or rectal administration of a pharmaceutical composition can include, for example, suppositories, which consist of the pharmaceutical composition with a suppository base. Suitable suppository bases include natural or synthetic triglycerides or paraffin hydrocarbons. In addition, it is also possible to use gelatin rectal capsules, which consist of a combination of the pharmaceutical composition with a base, including, for example, liquid triglycerides, polyethylene glycols, and paraffin hydrocarbons.

### Components of Immunogenic Compositions

In certain embodiments, immunogenic compositions, including e.g., one or more transformed plants or a pharmaceutical composition comprising an antigen purified from transformed plant cells, may be formulated as described above and/or additionally with one or more additional components. In some embodiments an additional component may be or comprise one or more of the following: an adjuvant, stabilizer, buffer, surfactant, controlled release component, salt, preservative, and an antibody specific to said antigen.

Adjuvants

In some embodiments, an immunogenic composition and/or a transformed plant can include or be administered with an adjuvant. In some embodiments where the immunogenic composition comprises one or more transformed plants, the transformed plant cells, containing lignins and HSPs (heat shock proteins) can act as an adjuvant in a subject (e.g., a non-human animal) being administered the immunogenic composition. For example, plant species such as sorghum and millet contain high quantities in saponins, and can act as an adjuvant in a subject being administered an immunogenic composition comprising transformed sorghum or millet.

In some embodiments, immunogenic compositions may additionally include or be administered with a biological adjuvant. Examples of biological adjuvants can include cholera toxin subunit B (CTB), hepatitis B virus core antigen (HBcAg), Escherichia coli heat labile enterotoxin subunit B (LTB), and monophosphoryl lipid A.

In some embodiments, an adjuvant can include inorganic adjuvants. Examples of inorganic adjuvants include alum salts such as aluminum phosphate, amorphous aluminum hydroxyphosphate sulfate, and aluminum hydroxide.

In some embodiments, an adjuvant can include a saponin. Typically, a saponin is a triterpene glycoside, such as those isolated from the bark of the Quillaja saponaria tree. A saponin extract from a biological source can be further fractionated (e.g., by chromatography) to isolate the portions of the extract with the best adjuvant activity and with acceptable toxicity. Typical fractions of extract from Quillaja saponaria tree used as adjuvants are known as fractions A and C. An exemplary saponin adjuvant is QS-21, which is available from Antigenics. QS-21 is an oligosaccharide-conjugated small molecule. Optionally, QS-21 may be admixed with a lipid such as 3D-MPL or cholesterol.

A particular form of saponins that may be used in immunogenic compositions described herein is immunostimulating complexes (ISCOMs). ISCOMs are an art-recognized class of adjuvants, that generally comprise Quillaja saponin fractions and lipids (e.g., cholesterol and phospholipids such as phosphatidyl choline).

In some embodiments, an adjuvant can include a TLR (Toll-like receptor) ligand. TLRs are proteins that may be found on leukocyte membranes, and recognize foreign antigens (including microbial antigens). An exemplary TLR ligand is IC-31, which is available from Intercell. IC31 comprises an anti-microbial peptide, KLK, and an immunostimulatory oligodeoxynucleotide, ODN1a. IC31 has TLR9 agonist activity. Another example is CpG-containing DNA, and different varieties of CpG-containing DNA are available from Prizer (Coley): Vaxlmmune is CpG 7909 (a (CpG)-containing oligodeoxy-nucleotide), and Actilon is TLR9 agonist, CpG 10101 (a (CpG)-containing oligodeoxy-nucleotide).

In some embodiments, an immunogenic composition (e.g., a pharmaceutical composition as described above) may include adjuvants that are covalently bound to antigens (e.g., purified from transformed plants, as described above). In some embodiments, an adjuvant can be recombinantly fused with an antigen. Other exemplary adjuvants that may be covalently bound to an antigen include, without limitation, polysaccharides, synthetic peptides, lipopeptides, and nucleic acids.

In some embodiments, an adjuvant can be co-expressed and part of the exogenous nucleic acid sequence encoding an antigen. In some embodiments, an adjuvant, can be co-expressed in a transformed plant cell with any antigen of interest (e.g., using a 2A sequence).

An adjuvant can be included in or administered with an immunogenic composition alone or in combination with another adjuvant. Adjuvants may be combined to increase the magnitude of the immune response to the antigen. In some embodiments, the same adjuvant or mixture of adjuvants is present in each dose of immunogenic composition. In some embodiments, an adjuvant may be administered with the first dose of immunogenic composition and not with subsequent doses. In some embodiments, a strong adjuvant may be administered with the first dose of immunogenic composition and a weaker adjuvant or lower dose of the strong adjuvant may be administered with subsequent doses. An adjuvant can be administered before the administration of an immunogenic composition, concurrent with the administration of an immunogenic composition or after the administration of an immunogenic composition to a subject (sometimes within 1, 2, 6, or 12 hours, and sometimes within 1, 2, or 5 days). Certain adjuvants are appropriate for human patients, non-human animals, or both.

Additional Components of Immunogenic Compositions

In some embodiments, an immunogenic composition, including e.g., pharmaceutical compositions, may include one or more optional additional components.

In some embodiments, an immunogenic composition can include one or more stabilizers such as sugars (such as sucrose, glucose, or fructose), phosphate (such as sodium phosphate dibasic, potassium phosphate monobasic, dibasic potassium phosphate, or monosodium phosphate), glutamate (such as monosodium L-glutamate), gelatin (such as processed gelatin, hydrolyzed gelatin, or porcine gelatin), amino acids (such as arginine, asparagine, histidine, L-histidine, alanine, valine, leucine, isoleucine, serine, threonine, lysine, phenylalanine, tyrosine, and the alkyl esters thereof), inosine, or sodium borate.

In some embodiments, an immunogenic composition can include one or more buffers such as a mixture of sodium bicarbonate and ascorbic acid. In some embodiments, the vaccine formulation may be administered in saline, such as phosphate buffered saline (PBS), or distilled water. In certain embodiments, an immunogenic composition includes one or more salts such as sodium chloride, ammonium chloride, calcium chloride, or potassium chloride. In certain embodiments, a preservative is included in the immunogenic composition. In other embodiments, no preservative is used. In certain embodiments, a preservative is 2-phenoxyethanol, methyl and propyl parabens, benzyl alcohol, and/or sorbic acid.

In certain embodiments, an immunogenic composition or pharmaceutical composition is a controlled-release formulation.

### Administration

Various methods of administering a transformed plant and/or a particular immunogenic composition (e.g., a plant-based vaccine) to a subject, (e.g., a non-human animal such as a ruminant livestock) can be used.

### Routes of Administration

In some embodiments, a transformed plant (e.g., a plant expressing an exogenous nucleic acid sequence encoding an antigen of interest) or an immunogenic composition (e.g., a plant-based vaccine) is fed to a non-human animal (e.g., a livestock animal).

In some embodiments, immunogenic compositions herein can be delivered by administration to an individual, typically by systemic administration (e.g., intravenous, intraperitoneal, intramuscular, intradermal, subcutaneous, transdermal, subdermal, intracranial, intranasal, mucosal, anal, vaginal, oral, sublingual, buccal route or they can be inhaled) or they can be administered by topical application.

In some embodiments, an immunogenic composition can be administered via the intramuscular route. Typically, in this route, the vaccine is injected into an accessible area of muscle tissue. Intramuscular injections are, in some embodiments, given in the deltoid, vastus lateralis, ventrogluteal or dorsogluteal muscles. The injection is typically given at an approximately 90° angle to the surface of the skin, so the vaccine penetrates the muscle.

An immunogenic composition may also be administered subcutaneously. The injection is typically given at a 45° angle to the surface of the skin, so the vaccine is administered to the subcutis and not the muscle.

In some embodiments, an immunogenic composition is administered intradermally. Intradermal administration is similar to subcutaneous administration, but the injection is not as deep and the target skin layer is the dermis. The injection is typically given at a 10-15° angle to the surface of the skin, so the vaccine is delivered just beneath the epidermis.

### Timing of Administration

In some embodiments, a transformed plant is harvested and included in a formulation or feed composition before administration. In some embodiments, a transformed plant may be produced to stably express an antigen of interest, and is then harvested and further cultivated in order to generate progeny expressing the antigen of interest (e.g. a leukotoxin A protein).

In some embodiments, a non-human animal self-administers a transformed plant and/or immunogenic composition, e.g., is subject to grazing the transformed plant and/or immunogenic composition. In some embodiments, where a transformed plant is transiently expressing an antigen of interest, expression of the antigen sequence may be tested before administration.

In some embodiments, administration may be or comprise one or more doses of a transformed plant and/or immunogenic composition. By way of specific example, a non-human animal may be administered (e.g., fed) the transformed plant multiple time over an extended period of time. In some embodiments, an extended period of time may be a period of time that is greater than 1 hour, 2 hours, 4 hours, 8 hours, 12 hours, 24 hour, or 1, 2, 3, 4, 5, 6, or 7 days. In some embodiments, administration of the transformed plant and/or immunogenic composition occurs over a period of 1, 2, 3, 4, 5, 6, 7 days, or more.

In some embodiments, a non-human animal is administered (e.g., fed) a transformed plant and/or immunogenic composition over a period time of greater than 1, 2, 3, 4, 5, 6, 7, 8, 9, 10, 11, or 12 weeks (e.g., consecutive weeks). In some embodiments, a non-human animal is administered (e.g. fed) a transformed plant and/or immunogenic composition hourly, daily, multiple times a day (e.g., 2-4), weekly, monthly, or yearly. In some embodiments, a non-human animal is administered a transformed plant and/or immunogenic composition for 1 or 2 days per week. In some embodiments, a non-human animal is administered (e.g. fed) a transformed plant and/or immunogenic composition at least 1, 2, 3, 4, 5, 6, or 7 days per month (e.g., consecutive days). In some embodiments, only one dose of the transformed plant and/or immunogenic composition (e.g., plant-based vaccine) is administered to achieve the results described above. In other embodiments, following an initial dosing, subjects receive one or more additional doses, for a total of two, three, four or five doses. A second or additional dose may be administered, for example, about 1 month, 2 months, 4 months, 6 months, or 12 months after the initial dose, for example, one dosing regimen can involve administration at 0, between 0.5-2, and between 4-8 months. It may be advantageous to administer split doses of an immunogenic composition by the same or different routes.

In some embodiments, a non-human animal is administered (e.g., fed) a transformed plant and/or immunogenic composition continuously (e.g., allowed to graze continually). In some embodiments, a non-human animal is (e.g., fed) a transformed plant and/or immunogenic composition continuously for at least 1 hour, 2 hours, 4 hours, 8 hours, 12 hours, 24 hour, or 1, 2, 3, 4, 5, 6, or 7 days. In some embodiments, a non-human animal is (e.g., fed) a transformed plant and/or immunogenic composition continuously for at least 1, 2, 3, 4, 5, 6, 7, 8, 9, 10, 11, or 12 weeks. As used herein, the term “continuously” means each meal of a particular hour, day, or week.

In some embodiments, a dose is administered (i.e., fed) to a non-human animal in a specified amount of feed or a non-human animal is allowed to feed for a specified period of time (i.e., “pulse” feeding). In some embodiments, a pulse feeding regimen includes weekly one-day pulses (e.g., at day 0, day 7, and day 14).

In some embodiments, a treatment regimen comprises a first dose of transformed plant and/or immunogenic composition (e.g., a plant-based vaccine) followed by a second, third or fourth dose. In some embodiments, a first dose of immunogenic composition comprises an immunogenic composition that contains one or more antigens of interest, or nucleic acids encoding one or more antigens of interest, or a combination of one or more antigens of interest and nucleic acids encoding the same or other antigens of interest. In some embodiments, a dose is formulated with the same antigens of interest, nucleic acids encoding the same, or a combination as the first dose. In some embodiments, a second or additional dose is formulated with different antigens of interest, nucleic acids encoding the same, or a combination with different antigens from the first dose. In some embodiments, an adjuvant is delivered concurrently or sequentially with one or more doses of transformed plant and/or immunogenic composition (e.g., a plant-based vaccine).

### Dosing

In some embodiments, the appropriate amount of antigen to be delivered will depend on the age, weight, and health (e.g., immunocompromised status) of a subject (e.g., a non-human animal such as a ruminant livestock).

Immunogenic compositions (e.g., plant-based vaccines) as described herein may take on a variety of dosage forms. In certain embodiments, the composition is provided in solid or powdered (e.g., lyophilized) form; it also may be provided in solution form. In certain embodiments, a dosage form is provided as a dose of lyophilized composition and at least one separate sterile container of diluent.

In some embodiments, a dose of immunogenic composition is calculated based on the amount of antigen desired to be delivered to a subject (i.e., a non-human animal). In some embodiments, and antigen is formulated in an amount of 1 μmol per dose. In some embodiments, the antigen is delivered at a dose ranging from 10 nmol to 100 nmol per dose. The appropriate amount of antigen to be delivered may be determined by one of skill in the art. In some embodiments, the appropriate amount of antigen to be delivered will depend on the age, weight, and health (e.g., immunocompromised status), and species of a non-human animal subject.

Immunogenic compositions disclosed herein are (in some embodiments) administered in amounts sufficient to elicit production of antibodies as part of an immunogenic response. In some embodiments, the composition may be formulated to contain 5 μg/0.5 ml or an amount ranging from 10 μg/1 ml to 200 μg/1 ml of an antigen. In other embodiments, the composition may comprise a combination of antigens. The plurality of antigens may each be the same concentration, or may be different concentrations. In some embodiments, immunogenic compositions formulated as plant-based vaccines will include a higher amount and/or concentration of antigen than an immunogenic composition formulated as a conventional vaccine or pharmaceutical composition. In some embodiments, immunogenic compositions formulated as plant-based vaccines will include at least 2×, 3×, 4×, or 5× the amount and/or concentration of antigen than an immunogenic composition formulated as a conventional vaccine or pharmaceutical composition. In some embodiments, the composition may be formulated as a ration of feed to be administered (i.e., fed) to a non-human animal. In some embodiments, the antigen(s) concentration to be included in the ration is based on antigen concentration as a percentage of total soluble protein in the ration. In some embodiments, a ration or composition includes an amount of antigen that is at least about 0.1% of the total soluble protein in the ration or composition. In some embodiments, a ration or composition includes an amount of antigen that is at least about 0.1%, 0.2%, 0.3%, 0.4%, 0.5%, 0.6%, 0.7%, 0.8%, 0.9%, 1,0%, 2.0%, 3.0%, 4.0%, or 5.0% or more of the total soluble protein in the ration or composition. In some embodiments, the amount of antigen in a ration or composition is within the range of about 0.5% to about 2% of the total soluble protein in the ration or composition.

In some embodiments, an immunogenic composition will be administered in a dose escalation manner, such that successive administrations of the immunogenic composition contain a higher concentration of composition than previous administrations. In some embodiments, an immunogenic composition will be administered in a manner such that successive administrations of an immunogenic composition contain a lower concentration of composition than previous administrations.

In some embodiments, only one dose (administration) of an immunogenic composition is administered. In other embodiments, the immunogenic composition is administered in multiple doses and/or multiple times. In various embodiments, the immunogenic composition is administered once, twice, three times, or more than three times. The number of doses administered to a subject can be dependent upon, for example, the antigen in the immunogenic composition, the extent of the disease or the expected exposure to the disease, and the response of a subject (e.g, a non-human animal) to the composition.

Use of Transformed Plants and/or Immunogenic Compositions

In some embodiments, a transformed plant and/or immunogenic composition (e.g., plant-based vaccine) described herein, may be used for prophylactic and/or therapeutic treatment of an infection (e.g., an infection caused by Fusobacterium). Use of transformed plants and/or immunogenic compositions may depend, for example, on many factors, including without limitation, the age, weight, and health (e.g., immunocompromised status) of a subject (e.g., a non-human animal such as a ruminant livestock), whether or not the subject has been exposed to a particular antigen, the symptoms or lack of symptoms presented by a subject, and other diseases present in the subject.

### Prophylactic Use

In prophylactic embodiments, a transformed plant and/or immunogenic composition described herein (e.g., plant-based vaccine) is administered to a subject to induce an immune response that can help protect against an infection (e.g., an infection common to livestock animals, e.g., Fusobacterium infection causing ruminal acidosis, rumenitis, and liver abscess).

In some embodiments, a transformed plant and/or immunogenic composition confers protective immunity, allowing a subject (e.g., a ruminant animal) to exhibit delayed onset of symptoms or reduced severity of symptoms of an infection as the result of exposure to the a transformed plant and/or immunogenic composition (e.g., a memory response). In certain embodiments, the reduction in severity of symptoms is at least 5%, 10%, 15%, 20%, 25%, 40%, 50%, 60%, 70%, 80% or even 90%. Some animals who have been administered a transformed plant and/or immunogenic composition of the present disclosure, may display no symptoms upon contact with and antigen, e.g., Fusobacterium, or even no infection by e.g., a Fusobacterium infection. In some embodiments, the IgG titer in serum of an animal that has been administered an a transformed plant and/or immunogenic composition can be raised by 1.5-fold, 2-fold, 3-fold, 4-fold, 5-fold, 10-fold, 20-fold, 50-fold, or even 100-fold or more following administration of a vaccine formulation described herein. In certain embodiments, the amount of IFN-γ released is 1.5-fold, 2-fold, 3-fold, 4-fold, 5-fold, 10-fold, 20-fold, 50-fold or even 100-fold greater. In some embodiments, the protective immunity conferred by presentation of antigen before exposure to said antigen will reduce the likelihood of a future infection.

The duration of protective immunity may vary in accordance with various embodiments. In some embodiments, protective immunity lasts for six months, one year, two years, five years, ten years, twenty years or even a lifetime. In some embodiments, protective immunity lasts only hours, days, or weeks (e.g., 1 day, 2 days, 3 days, 4 days, 5 days, 6 days, one week, two week, three weeks).

In some embodiments, a combination of specific polypeptide antigens (e.g., immunogenic fragments of ltkA) may prove efficacious for treating an infection (e.g., a Fusobacterium infection) or the onset of symptoms described above. An exemplary immunogenic composition (e.g., plant-based vaccine) for prophylactic use may comprise a carrier, any combination of immunogenic fragments of ltkA selected from PL1, PL2, PL3, PL4, and PLS, or any fragment or variant thereof.

### Therapeutic Use

In some embodiments including therapeutic applications, a transformed plant and/or immunogenic composition (e.g., a plant-based vaccine) comprising an antigen and/or nucleic acid encoding an antigen described herein may be administered to a non-human animal subject suffering from a disease, disorder, or condition (e.g., an infection common to livestock animals, e.g., a Fusobacterium infection) in an amount sufficient to treat the subject (e.g., a non-human animal, such as ruminant livestock). Treating the subject, in this case, may refer to delaying and/or reducing one or more symptoms of an infection. In certain embodiments, administration of a transformed plant and/or immunogenic composition as provided for herein, may result in the reduction of one or more symptoms by at least 5%, 10%, 20%, 25%, 30%, 40%, 50%, 60%, 70%, 80% or even 90%, as compared to the subject, prior to receiving the transformed plant and/or immunogenic composition.

The timing of administration of a first (or subsequent) dose of a plant or composition as provided for herein may vary in an application-appropriate manner (e.g., relative to timing of infection or symptom presentation). For example, an immunogenic composition may be administered shortly after infection, e.g. before symptoms manifest, or may be administered during or after manifestation of symptoms. In some embodiments, a transformed plant and/or immunogenic composition may prevent endogenous reactivation of earlier infection.

In some embodiments, a transformed plant and/or immunogenic composition is administered in an amount that results in an immune response in a non-human animal. Methods of assessing immune response include, e.g., testing antibody response. In some embodiments, antibody response is measured by obtaining serum from a non-human animal, (e.g., a non-human animal suffering from or susceptible to a Fusobacterium infection) treated with a transformed plant and/or immunogenic composition, and measuring antibodies specific for a particular antigen (e.g., a virulence factor of Fusobacterium or an immunogenic fragment thereof). In some embodiments an antibody titer assay (e.g., ELISA) is used to detect antibodies.

In some embodiments, an immune response is measured by determining level of expression and/or secretion of various inflammatory markers (e.g., haptoglobin, serum-amyloid A, fibrinogen, interleukin-6, and tumor necrosis factor-α).

### Transformed Plants for Treatment of Fusobacterium Infection

In some embodiments, a transformed plant and/or immunogenic composition of the disclosure can be used to treat a Fusobacterium infection. Fusobacterium is a genus of gram negative, anaerobic bacteria that includes several species, including Fusobacterium necrophorum and Fusobacterium nucleatum. Fusobacterium nucleatum is the most frequently isolated species from humans and includes 5 subspecies.

Fusobacterium necrophorum includes two subspecies (F. necrophorum ssp. necrophorum, and F. necrophorum ssp. funduliformis). F. necrophorum is frequently isolated from non-human animals and can cause a variety of infections (e.g. foot rot, hepatic abscess, stomatitis, and gangrenous dermatitis). In humans, a F. necrophorum commonly presents as “Lemierre's syndrome” (postanginal sepsis). Other infections caused by F. necrophorum include liver abscess, lung abscess, infections of the female genital tract, intra-abdominal infections, and skin-structure infections. The typical virulent diseases in animals and humans are caused by F. necrophorum ssp. necrophorum, (Citron, Diane M. Clinical infectious diseases 35.Supplement_1 (2002): S22-S27).

Colonization of F. necrophorum bacteria can lead to symptoms such as footrot, Footrot (in e.g., cattle or sheep) is caused by colonization of F. necrophorum bacteria in the area of a trauma site to the foot followed by exposure to a wet or damp environment and is characterized by painful inflammation of the interdigital skin of the infected animal. Implications of the disease include lameness, loss of appetite, loss of weight, and mortality.

F. necrophorum is additionally known to cause symptoms such as liver abscesses, which can result from an ulcerated rumen, through which the F. necrophorum bacteria (in some cases residing in the microflora of the gastrointestinal tract) travel to the bloodstream, and continue through the portal vein to invade the liver and cause an abscess.

In some embodiments, a transformed plant that is used to treat a Fusobacterium infection is a transformed plant that expresses an exogenous nucleic acid sequence encoding leukotoxin A, or a fragment or variant thereof. In some embodiments, a transformed plant that is used to treat a Fusobacterium infection is a transformed plant that expresses an exogenous nucleic acid sequence encoding another virulence factor of Fusobacterium, or a fragment or variant thereof. In some embodiments, a transformed plant and/or immunogenic composition confers protective immunity, allowing a subject (e.g., a ruminant animal) to exhibit delayed onset of symptoms or reduced severity of symptoms of an Fusobacterium infection as the result of exposure to the a transformed plant and/or immunogenic composition (e.g., a memory response). In some embodiments, a transformed plant and/or immunogenic composition results in increased weight/body mass of a subject (e.g., a ruminant animal).

In some embodiments including therapeutic applications, a transformed plant and/or immunogenic composition (e.g., a plant-based vaccine) comprising an antigen such as leukotoxin A and/or nucleic acid encoding an antigen described herein may be administered to a non-human animal subject suffering from a symptom resulting from a Fusobacterium infection in an amount sufficient to treat the subject (e.g., a non-human animal, such as ruminant livestock).

Other features of the invention will become apparent in the course of the following descriptions of exemplary embodiments, which are given for illustration of the invention and are not intended to be limiting thereof.

### EXAMPLES

The following examples disclose exemplary methods of transforming plant cells (e.g., from sorghum or millet plant species) with a nucleic acid material (e.g., a DNA construct) including an exogenous nucleic acid sequence encoding one or more immunogenic fragments of leukotoxin A. Methods described below further include formulating the transformed plant into an immunogenic composition and administering the immunogenic composition to a non-human animal subject (e.g. a ruminant livestock) suffering from a disease, disorder, or condition resulting from a Fusobacterium infection.

### Example 1: Nucleic Acid Constructs

The examples below utilize two immunodominant regions of leukotoxin, namely PL1 and PL4, to develop selected crop species that synthesize these proteins such that they may be used as a plant-based vaccine for pasture ruminants. For the purposes of this example, the chloroplast of sorghum (Sorghum bicolor (L.) Moench, Genbank: NC_008602.1) and the chloroplast of millet (Panicum miliaceum L., GenBank: KU343177.1) were selected as host plastomes for synthesizing exemplary plant-based vaccines. Three immunogenic protein operons will be produced by the sorghum plastome: PL1 (SEQ ID NO: 4), PL4 (SEQ ID NO: 10), and PL1+PL4. The additive effect of a plant transformed with a combination will be examined by comparing the plant producing both PL1 and PL4, compared to a plant transformed with only one of PL1 or PL4, to see if there are additive effects of having multiple immunogenic protein fragments in the plant vaccine. In this example, only the PL1+PL4 operon will be introduced into the millet chloroplast. Having decided the host species, what follows is the description of the DNA construct necessary to express an antigen in each the host species' plastomes.

### Targeting Sequences

In order to provide a successfully transformed and productive plant (e.g., for a plant-based vaccine), several variables must be considered. By way of non-limiting example, selection of a proper chromosomal location is critical, inter alia, to ensure normal gene expression occurs with minimal or no disruption, and also to ensure that therapeutic levels of the exogenous nucleic acid are produced. In addition, other factors, such as the length of the targeting sequence, can affect the efficiency and accuracy of the transformation. In this Example, the chloroplast genome of sorghum was selected for analysis. The sorghum chloroplast (Genbank: NC_008602.1, Saski et al., 2007) was analyzed and a region between the trnG-UCC and trnM-CAU genes was selected for transgenic insertion. Specifically, chloroplast bases 14048 through 14793 and 14794 through 15561 were designated as the ‘left flank’ and ‘right flank’ for the nucleic acid construct, respectively.

The millet chloroplast (GenBank: KU343177.1) was analyzed and a region between the trnY-GUA and trnD-GUC genes was identified for transgenic insertion. Also, millet chloroplast bases 16408 through 16845, and 16846 through 17960 were designated as the ‘left flank’ and ‘right flank’, respectively. These flanking regions will facilitate the homologous recombination to maneuver the exogenous nucleic acid sequence into the chloroplast genome. Regions of the sorghum and millet chloroplast genome were identified and based on regions known or suspected to be involved in tRNA synthesis. Further, regions were selected for having large spans of sequences unlikely to include a promoter, enhancer, and terminator sequence.

### Exogenous Nucleic Acid Sequence

As is known in the art, fusobacterium infection in ruminant livestock can lead to a number of symptoms, including ruminal acidosis, rumenitis, and liver abscess, and presents a costly problem for the livestock industry. Immunodominant Fusobacterium leukotoxin (Genbank: DQ672338) regions PL1 and PL4 were selected as antigens of interest to be formulated in a plant-based vaccine. PL1 and PL4 DNA sequences were translated separately in silico and their respective sequences were amended to include in-frame start (ATG) and stop (TAA) codons, to ensure correct genetic transcription of these sequences.

### Selection Sequence

In order to assess whether and how plants are transformed, as well as to assess the level of expression of the exogenous nucleic acid sequence, one or more selection sequences were used in this example. For ease of assessment, fluorescent selection sequences were used in this Example, though this need not always be the case. Specifically, three fluorescent proteins were selected to discretely confirm the expression of each of three immunogenic protein operons:
Yellow fluorescence protein (YFP, GenBank: GQ221700.1 or SEQ ID NO: 6) for PL1;
Red fluorescence protein (DsRED, GenBank: KY426960.1 or SEQ ID NO: 12) for PL4; and
Cyan fluorescence protein mTurquoise2 (CFP, GenBank: HQ993060.1 or SEQ ID NO: 14) for PL1+PL4.

### Enhancer Sequence

In order to increase transcription of the exogenous nucleic acid sequence, certain enhancer sequences were selected. Enhancer sequences are positioned relative to a promoter sequence and the antigen of interest to be expressed (in this example, PL1, PL4 or PL1+PL4). Each antigen to be expressed will be equipped with its own leader sequence:
T7phage genel0 leader sequence (Olins et al., 1988, GenBank: EU520588.1 or SEQ ID NO: 3) for PL1;
Bacillus thuringiensis Lcry9Aa2 gene leader (GenBank:MF461355.1 or SEQ ID NO: 5) for YFP;
Tobacco LrbcL leader (GenBank:EU224430.1 or SEQ ID NO: 9) for PL4;
Tobacco LatpB leader (GenBank:DQ672338.1 or SEQ ID NO: 11) for DsRED; and
Tobacco mosaic virus omega prime translation leader (GenBank: KM507060.1 or SEQ ID NO: 13) for CFP.

### Promoter Sequence

In addition to an enhancer sequence, the DNA constructs used in this example include a promoter sequence in proximity (upstream) of the 5′ end of the exogenous nucleic acid sequence, to initiate transcription of the antigen (in this example, PL1, PL4 or PL1+PL4). A single constitutively expressed rRNA promoter Prrn from tobacco (GenBank: MF580999.1 or SEQ ID NOs: 2, 21) or PpsbA (SEQ ID NO: 24) was selected and shown to be successful in synthesizing polycistronic operons in several plant species, including Arabidopsis.

### Termination Sequence

In the DNA construct of this example, a single terminator sequence was selected to cease transcription of the transgenic operon and to be placed within the DNA construct in a position relative to the exogenous nucleic acid sequence encoding the antigen (at the 3′ end of the sequence encoding PL1, PL4). Specifically, the tobacco gene rps16 (GenBank: MF580999.1 or SEQ ID NO: 7 or SEQ ID NO: 22) was selected as it has been successfully used in many chloroplast transformation vectors.

### Sorghum Nucleic Acid Constructs

The elements described above in this example were arranged and incorporated to form a DNA construct to be introduced (e.g., by transformation) into a host plant (in this particular example, the host plants are sorghum).

In this example, and in accordance with the above, three sorghum-targeted constructs were made:

Construct 1: Left Flank_{Sorghum}-Prrn_{Nicotiana}-Lgene10_{T7phage}-PL1_{Fusobacteria}-Lcry9Aa2_{Bacillus}-YFP-Trps16_{Nicotiana}Right Flank_{Sorghum}; 4022 bases (FIG. 1, SEQ ID NO: 17)

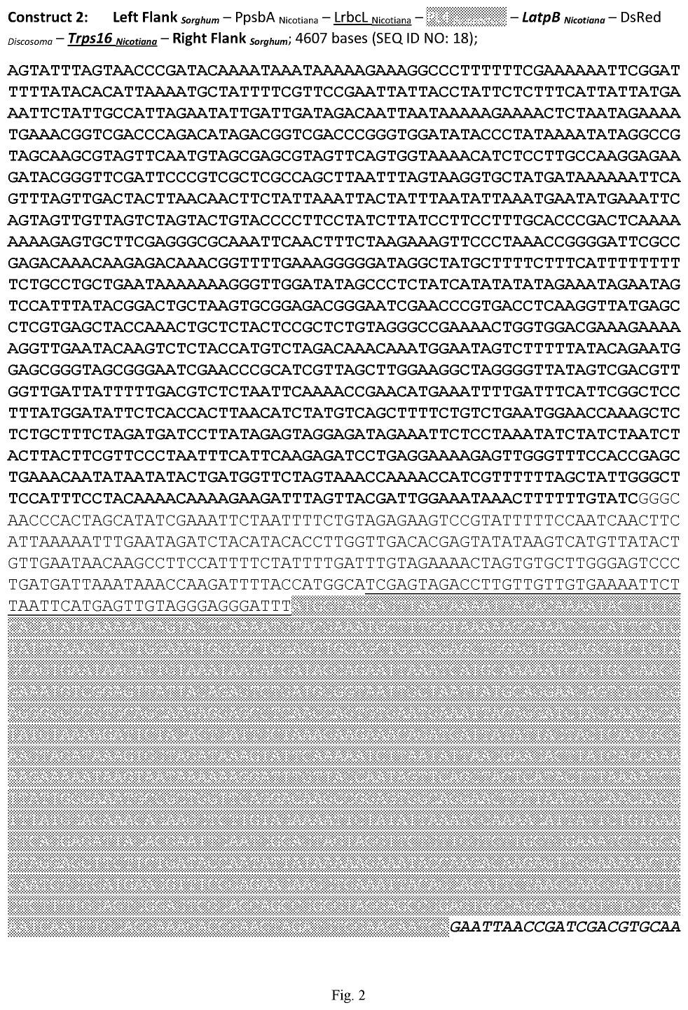

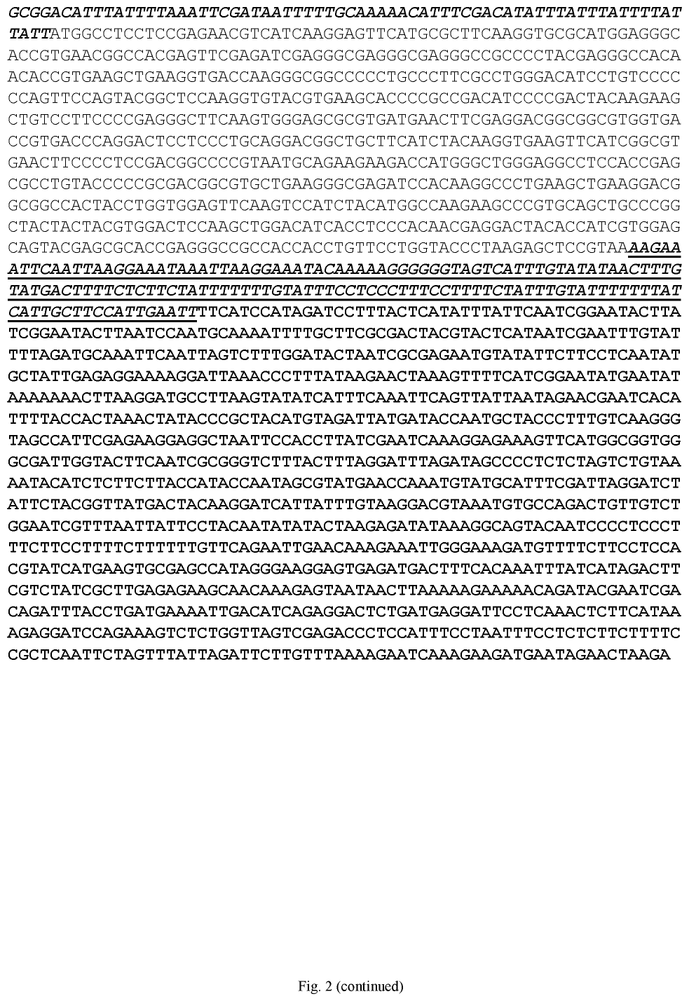

Construct 2: Left Flank_{Sorghum}-PpsbA_{Nicotiana}-LrbcL_{Nicotiana}-PL4_{Fusobacteria}-LatpB_{Nicotiana}-DsRed_{Discosoma}-[Trps16_{Nicotiana} or Trps16_{Nicotiana}alt]-[Right Flank_{Sorghum} or Right Flank_{Sorghum} alt] 4607 bases (FIG. 2; SEQ ID NO: 18)

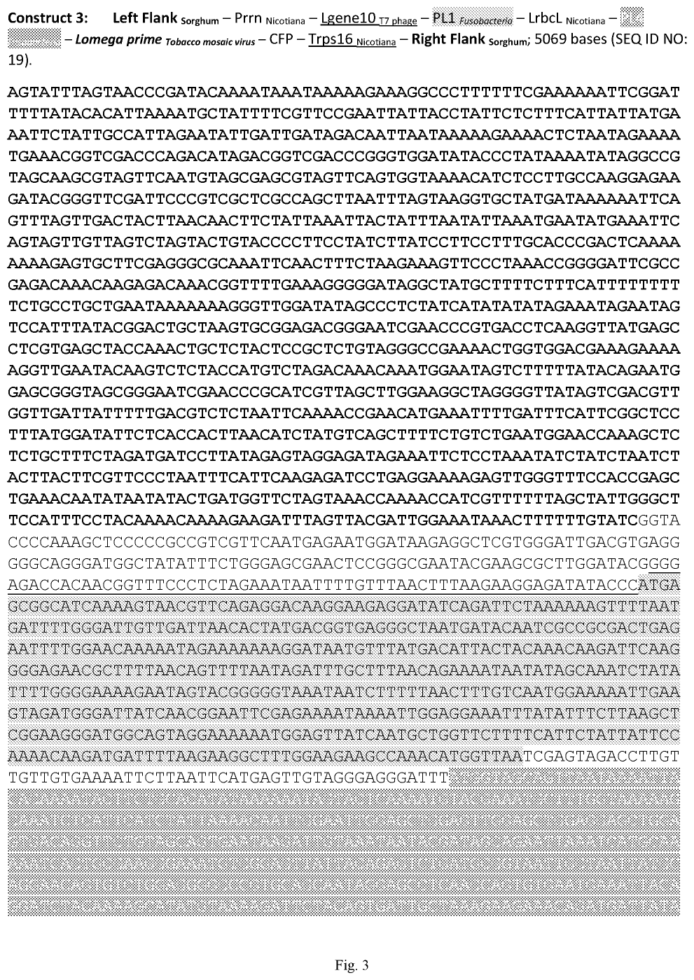

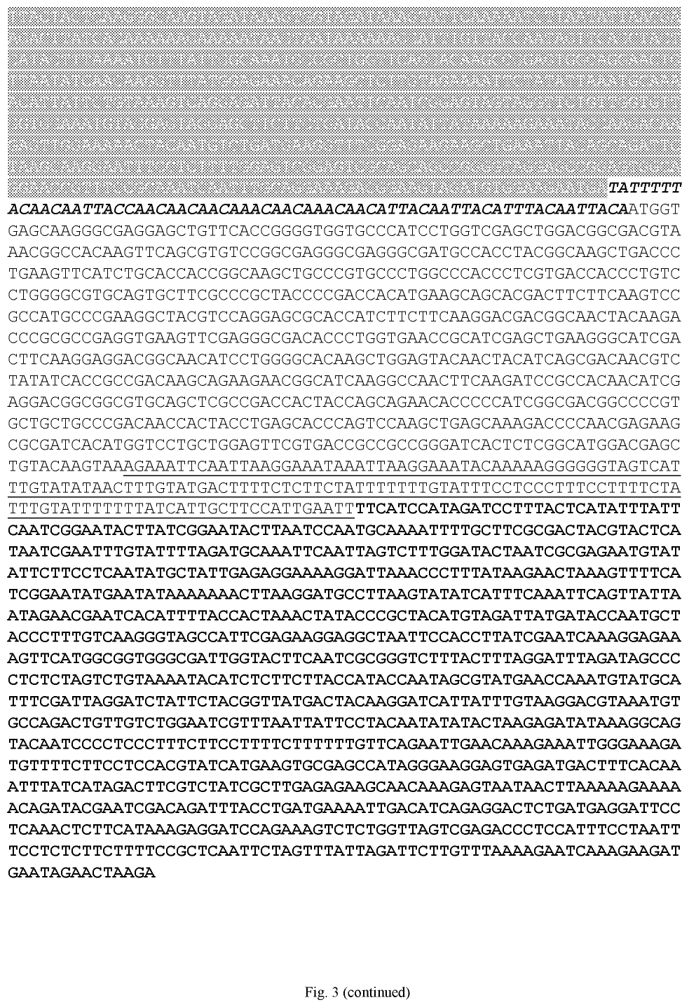

Construct 3: Left Flank_{Sorghum}-Prrn_{Nicotiana}-Lgene10_{T7phage}-PL1_{Fusobacteria}-LrbcL_{Nicotiana}-PL4_{Fusobacteria}-Lomega prime_{Tobacco mosaic virus}-CFP-Trps16_{Nicotiana}-Right Flank_{Sorghum} or Right Flank_{Sorghum} alt]; 5069 bases (FIG. 3, SEQ ID NO: 19)

### Millet Nucleic Acid Constructs

The elements described above in this example were arranged and incorporated to form a DNA construct to be introduced (e.g., by transformation) into a host plant (in this particular example, the host plants are millet).

In this example, and in accordance with the above, a millet-targeted construct was made:

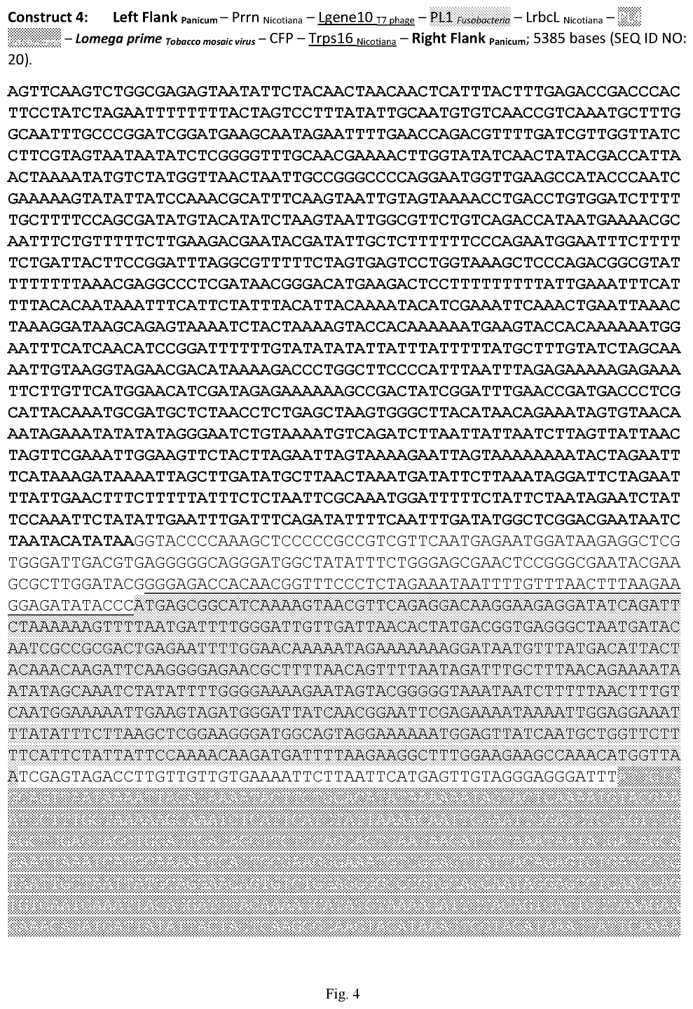

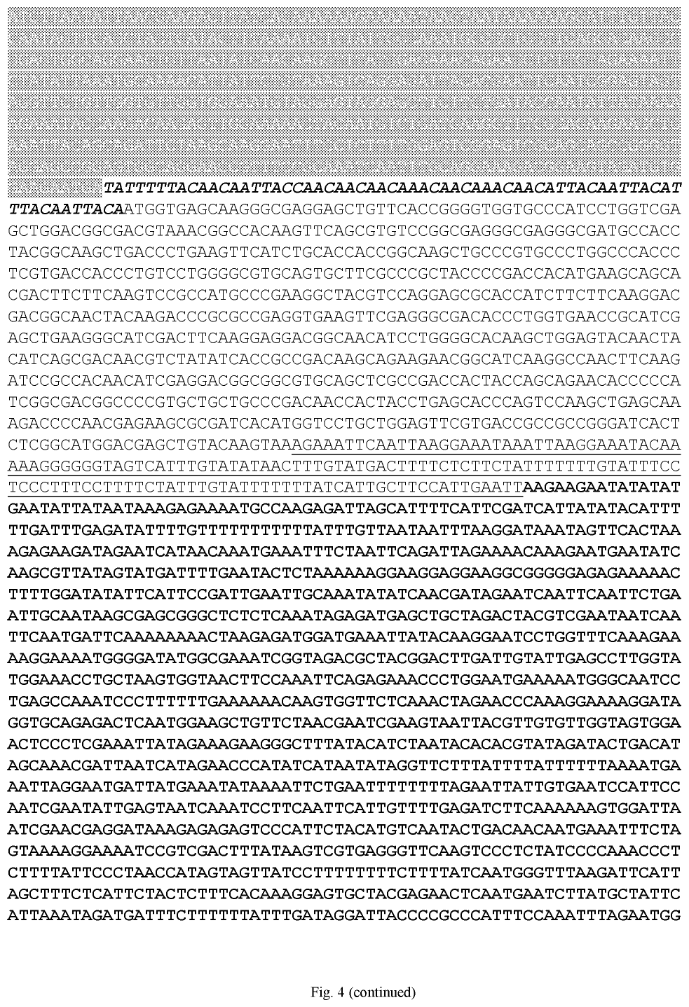

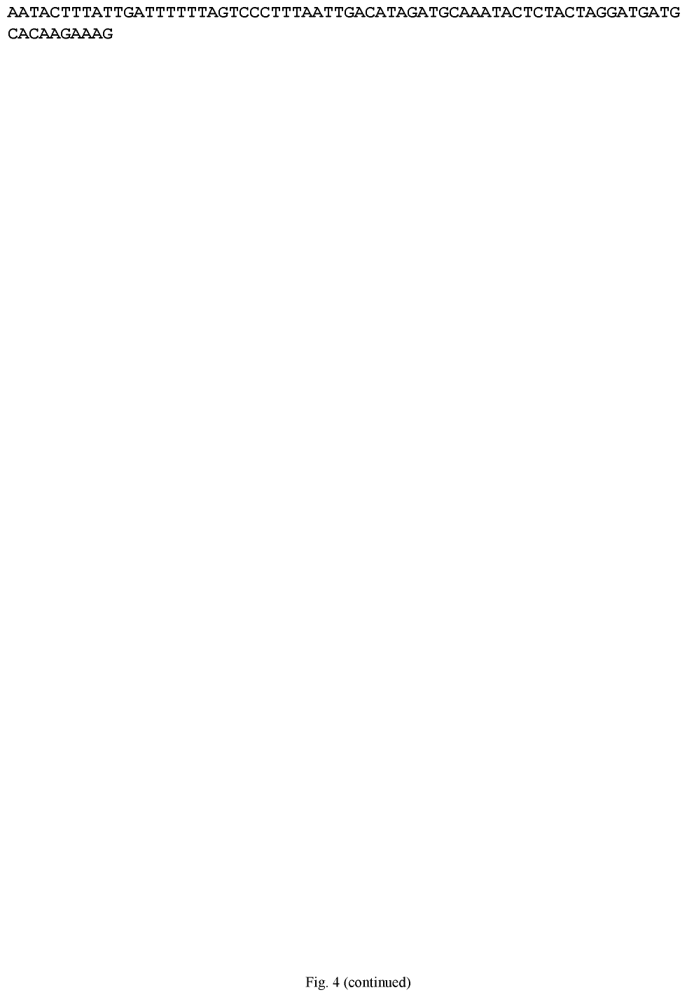

Construct 4: Left Flank_{Panicum}-Prrn_{Nicotiana}-Lgene10_{T7phage}-PL1_{Fusobacteria}-LrbcL_{Nicotiana}-PL4_{Fusobacteria}-Lomega prime_{Tobacco mosaic virus}-CFP-Trps16_{Nicotiana}-Right Flank_{Panicum}; 5940 bases. (FIG. 4, SEQ ID NO: 20)

### Example 2: Methods of Generating Nucleic Acid Constructs

In order to obtain sufficient copies of the DNA construct to be introduced into a host plant genome, the DNA construct was obtained and then copied using standard PCR reactions, to then be purified from the product and formulated to be delivered to a host plant genome.

The nucleic acid constructs were generated within the pMX vector plasmid through Invitrogen's GeneArt Gene Synthesis (www.thermofisher.com/ca/en/home/life-science/cloning/gene-synthesis/geneart-gene-synthesis) service. The dry DNAs supplied by the manufacturer will be resuspended to 100 ng DNA/μL 10 mM Tris, 1 mM EDTA pH 8.0.

An abundance of copies of each DNA construct will be generated by polymerase chain reaction (PCR) using specific forward and reverse primers (synthesized by Eurofins Genomics (Brussels, Belgium)) aligned to the 5′ end of the respective Left Flanking region and the 3′ of the Right Flanking region, respectively. The reaction components will be assembled as below:

| Reaction componenets | Final concentration | |
|---|---|---|
| Pyrococcus furiosus polymerase | 0.4 | units |
| 10X PCR Buffer (200 mM Tris HCl | 1x | |
| (pH 8.4), 500 mM KCl) | | |
| 10 mM dNTPs | 1 | mM |
| 50 mM MgCl2 | 50 | mM |
| Forward primer | 1 | pmol |
| Reverse primer | 1 | pmol |
| Plasmid DNA | 20 | ng |

| | |
|---|---|
| Double distilled H₂O | up to 20 μL |

Each PCR reaction targeting templates >1 kb will be thermocycled as described below in a BioRad CFX96 Optical Thermocycler (BioRad):

| Step | Number of cycles | Temperature (° C.) | Duration | |
|---|---|---|---|---|
| 1 | 1 | 98 | 1 | minute |
| 2 | 30 | 98 | 45 | seconds |
| | | 60 | 45 | seconds |
| | | 72 | 6 | minutes |
| 3 | 1 | 72 | 10 | minutes |

The resulting reactions will be size fractionated in 2% agarose and amplicons of appropriate sizes will be excised and cleaned using a QIAquick Gel Extraction Kit (Qiagen, Venlo, Netherlands) according to manufacturer's instructions. Cleaned amplicons will be sequence confirmed using the Applied Biosystems (AB) 3500XL capillary sequencer and analyzed using Sequence Analysis v5.4 software (ThermoFisher Scientific, Waltham, Mass.). At least 10 μg of each sequence-confirmed amplicons will be stored at 4° C. until processing.

### Example 3: Exemplary Delivery Methods

Once the DNA constructs are isolated and purified in amounts of, for example, 10 μg of each sequence, the DNA constructs (each of the four described above), are formulated with a carrier. The carrier, in this example, aids in the efficiency and accuracy of the transformation into the host plant cell.

In this example, single-walled carbon nanotubes (SWCNTs, Sigma) will be used to guide the construct to chloroplasts of sorghum and millet leaves using similar procedures to those described by Demirer et al. (2019). SWCNTs will be prepared in the following manner:
1. Resuspend dry SWCNT in water to a concentration of 1 mg/mL;
2. Add 1 mg/mL SWCNT to 2% sodium dodecyl sulfate:water (SDS);
3. Bath sonicate the mixture for 10 minutes (40% amplitude, ˜12 W);
4. Tip sonicate the mixture with a 6 mm tip fat 40% amplitude (˜12 W) for 60 minutes on ice;
5. Allow mixture to rest for 30 minutes at room temperature;
6. Centrifuge the mixture 16,100×g for 60 minutes; and
7. Transfer the supernatant to a fresh tube for spectral analysis using a UV-Vis-nIR spectrometer.

The desired concentration of SWCNT in the obtained supernatant is to be ˜1 mg SWCNT/mL 2% SDS (using the concentration of SWCNTs absorbance at 632 nm/extinction coefficient of 0.036). At least 1 mg of each sequence-confirmed amplicons will be stored at 4° C. until processing.

Once the mixture containing the SWCNTs is prepared, the SWCNTs can be contacted with and conjugated to the DNA constructs prepared above. The stored amplicons of the DNA constructs will be adsorbed onto the prepared SWCNTs by dialysis using a pore-sized dialysis cartridge (Slide-A-Lyzer, ThemorFisher) according to manufacturer's instructions, resulting in conjugated DNA-SWCNTs. Methods for conjugating the SWCNTs to the DNA constructs are as follows:
1. 1 ug of prepared SWCNT and 10 μg of prepared DNA construct will be added directly to the dialysis cartridge via syringe needle (provided);
2. Add 2% SDS until the dialysis cartridge is full;
3. Attach the cartridge to a float buoy (supplied);
4. Place dialysis cartridge with attached float buoy in a 1 L beaker filled with 0.1 M sodium chloride (NaCl) and magnetic stir bar; and
5. Magnetically stir dialysis cartridge continuously for four days and change the dialysis buffer daily.

Confirmation of DNA adsorption to SWCNTs will be conducted by comparing the infrared fluorescence of the DNA-conjugated SWCNTs to a control sample, being the SWCNTs dialyzed in the absence of the DNA constructs. DNA adsorption to the SWCNTs is observed by higher infrared fluorescence than the control sample.

### Example 4: Transformation of Chloroplasts

The prepared DNA constructs conjugated to the SWCNTs (DNA-SWCNT) are then to be formulated for introduction into a host plant cell (e.g., via transformation). In this example, the conjugated DNA-SWCNTs will be infiltrated into small, gently punctured abaxial surfaces of the plant leaf lamina (depending on the DNA construct, into plant leaves of millet or sorghum) using a pipette tip or razor. A total of 100 μL of conjugated DNA-SWCNT will be applied to the punctured areas using a needleless syringe by applying a gentle pressure. After 24-72 hours of homologous recombination, the success of transformation will be evaluated using:
Level of transgenic gene expression will be evaluated using quantitative real-time PCR, by extracting total RNA using the RNeasy plant mini kit (Qiagen), iScript cDNA synthesis kit (Bio-Rad) and Powerup SyBR green master mix (Applied Biosystems), and comparing quantification thresholds between reactions with gene specific primers to reactions with ‘housekeeping’ gene(s).
Fluorescence will be observed using a confocal microscope by excising a small section of the infiltrated leaf, placing it between glass slide and coverslip, and exposing slides to appropriate excitation wavelengths to observe the fluorescence of the selection sequence (YFP, DsRED, and CFP, depending on the construct).

The amount of antigen production from the transformed plant will be quantified using ELISA. Methods for quantifying the amount of antigen produced from the transformed plant include the following :
1. Fresh leaf tissue (100 mg) will be ground by motor and pistil;
2. Ground tissues will be resuspended in 500 μL of extraction buffer (100 mM NaH₂PO₄, 8 M Urea, and 0.5 M NaCl; pH 8);
3. A standard curve (1-10 μg) of pure recombinant PL1 and PL4 antigens, provided by ThermoFisher Scientific (www.thermofisher.com/ca/en/home/life-science/antibodies/primary-antibodies/polyclonal-antibodies), diluted in carbonate buffer (pH 9.6), will also be plated;
4. Samples will be placed in a microfuge tube and centrifuged at 14,000 rpm at 4° C. for 10 minutes.
5. Protein extractions (diluted in carbonate buffer) will be incubated in select ELISA plate wells overnight at 4° C.;
6. Wash plate with PBST (3.2 mM Na₂HPO₄, 0.5 mM KH₂PO_{4, 1.3} mM KCl, 135 mM NaCl, 0.05% Tween 20, pH 7.4.);
7. Block plate with 2% fat-free dry milk in carbonate buffer for 60 minutes;
8. Wash plate once with PBST;
9. Incubate plate with anti-PL1 and -PL4 polyclonal antibodies (1:500 2% fat-free dry milk) for 60 minutes;
10. Wash with PBST;
11. Incubate plate with secondary monoclonal antibodies (1:10000 2% fat-free dry milk) for 60 minutes;
12. Add 0.3 mg/L 2-20 Azino-bis-3 etilbenztiasoline-6-sulphuric acid (ABTS; Sigma, Missouri, USA) and 0.1 M citric acid, pH 4.35;
13. Using a Multiskan Ascent (Thermo Scientific, Massachusetts, USA) microplate reader, record the optical density at 405 nm; and
14. Expressed PL1 and PL4 will be quantified by comparing OD₄₀₅ in 100 mg of total protein to OD₄₀₅ of standard curve.

### Example 5: Chloroplast Transformation

In this example, host plant chloroplast genomes were transformed with PL1, PL4, and PL1+PL4, immunodominant regions of Fusobacterium necrophorum leukotoxin A for, among other things, the purposes of engineering an edible vaccine to protect grazing cattle from F. necrophorum leukotoxin A. Host chloroplasts genomes were targeted by inoculating functionalized single-walled carbon nanotubes pre-loaded with plastid expression cassettes. Initial results show successful cassette infusion, integration of plastid expression cassettes, and expression of F. necrophorum immunogenic subunit transcripts were observed.

### Host Plant Material

In this example, host plant cereal species Sorghum sudangrass (Sorghum bicolor ((L.) Moench)×(Sorghum×drummondii) (Nees ex. Steud.)) seeds were germinated in Jiffy Peat Pellets and one-week-old seedlings were transplanted to pots filled with Vigoro All-Purpose Potting Soil for three weeks in an ambient and naturally lit room.

### Construct Development

DNA sequences coding for two immunogenic subunits Fusobacterium necrophorum leukotoxin A, namely PL1 and PL4 (see Sun et al., 2009), were sourced from Genbank accession DQ672338 and correspond to SEQ ID NOs: 4 and 10, respectively.

Sorghum sudangrass chloroplast transformation vectors were designed to facilitate integration of transgenic material between trnG and trnM genes. Left and right flanking regions of the vector correspond to bases 13151 through 14490 and 14491 through 15560 of the Sorghum bicolor chloroplast complete genome (Genbank accession NC_008602). Expression vector “PL1” (which is shown in Example 1 as “Construct 1” and identified in SEQ ID NO: 17) was designed with tobacco promoter Prrn, identified in Genbank MF580999, to drive the polycistronic expression of PL1, equipped with enhancer T7phage gene10 leader sequence (Genbank accession EU520588), and yellow fluorescent protein (YFP; Genbank accession GQ221700), equipped with enhancer Bacillus thuringiensis cry9Aa2 gene leader (GenBank accession MF461355), and terminated with tobacco Trps16 (GenBank accession MF580999).

Expression vector “PL4” (which is shown in Example 1 as “Construct 2” and identified in SEQ ID NO: 18) was designed with tobacco promoter PpsbA, identified in Genbank DQ459069 to drive polycistronic expression of PL4, equipped with tobacco enhancer rbcL (Genbank accession EU224430), and red fluorescent protein (DsRED; Genbank accession KY426960), equipped with tobacco enhancer LatpB (GenBank accession EU224425), and terminated with tobacco Trps16.

Expression vector “PL1+PL4” (which is shown in Example 1 as “Construct 3” and identified in SEQ ID NO: 19) was designed with tobacco promoter Prrn to drive the polycistronic expression of PL1 and PL4, equipped with enhancers T7phage gene 10 and tobacco rbcL, respectively, and cyan fluorescent protein (MTurquoise2; Genbank accession HQ993060), equipped with the enhancer TMV omega prime translation leader (GenBank accession KM507060), and terminated with tobacco Trps16.

A millet chloroplast transformation vector were designed to integrate between genes trnT and trnL of the Genbank accession KU343177, where left and right flanking targeting regions correspond to bases 46391 through 47746 and 47747 through 49115, respectively. This polycistronic vector was designed to express both PL1 and PL4 (equipped with enhancers T7phage gene 10 and tobacco rbcL, respectively), along with cyan fluorescent protein (equipped with the enhancer TMV omega prime translation leader), and be driven by tobacco promoter Prrn, and have expression terminated by tobacco Trps16 (which is shown in Example 1 as “Construct 4” and identified in SEQ ID NO: 20).

Expression vector synthesis was outsourced to GenScript (New Jersey, U.S.A.). Each vector was received as dry DNA contained in a plasmid backbone. Expression vectors were PCR amplified in 50 μL reactions composed of Phusion U Hot Start DNA Polymerase (ThermoFisher, Massachusetts, U.S.A.), 1×PCR buffer, water, and 10 μM of sorghum or millet chloroplast primers (see Table 1.) to generate amplicons from 5 pg of sorghum or millet plasmid templates, respectively. These PCR reactions were conducted on a BioRad CFX 384 (BioRad Laboratories, California, U.S.A.) thermocycler using the following conditions: initial denaturation at 98° C. for three minutes, 98° C. for 10 seconds, 63° C. for 30 seconds, and 72° C. for 5 minutes, with a final extension of 72° C. for 10 minutes. Ten microliters of PCR product were size fractionated on a 1% gel, illuminated by GelStar Nucleic Acid Stain (ThermoFisher) on a Dark Reader Transilluminator (Clare Chemical Research, Colorado, U.S.A.). The remaining PCR products were cleaned with ExoSAP-IT PCR Product Cleanup Reagent (ThermoFisher).

TABLE 1
Primers used to amplify expression vectors.

| Primer | | Length |
|---|---|---|
| name | 5′-3′ Sequence | (bp) |
| Sorghum | GTT ACG ATT GGA AAT AAA CTT | 30 |
| Left | TTT TGT ATC | |
| flank | | |
| Sorghum | GAA TAA ATA TGA GTA AAG GAT | 32 |
| Right | CTA TGG ATG AA | |
| flank | | |
| Millet | GGC TCG GAC GAA TAA TCT AAT | 22 |
| Left | ACA TAT AA | |
| flank | | |
| Millet | CAT TTT CTC TTT ATT ATA ATA | 23 |
| Right | TTC ATA TAT ATT CTT CTT | |
| flank | | |

### Preparation of Nanomaterials

Single walled carbon nanotubes (SWCNTs) were functionalized with chitosan and non-covalently bonded M_{w} 5,000 polyethylene glycol using the methods described by Kwak et al. (2019). Briefly, 0.3% acetic acid was mixed with 0.3 g low molecular weight deacetylated chitosan (CS, Sigma, Missouri, U.S.A) and 0.15 g high-pressure carbon monoxide (HiPCO) synthesized SWCNTs (Nanointegris, Quebec, Canada). This mixture was placed in an ice bath and was tip-sonicated for 40 minutes at 40% amplitude, and was dialysed overnight with 100 kDa molecular weight cutoff membranes in deionized water. These CS-SWCNTs were centrifuged twice at 16,100 g for two hours. PEGylation of CS-SWCNT took place by mixing 0.1 equivalent HO-PEG5K-NHS (Sigma) with chitosan nanotubes for six hours at room temperature, followed by centrifugation at 16,100 g for 75 minutes. CS^{PEG5K}-SWCNTs were reconstituted in 2-(N-morpholino)ethanesulfonic acid (MES) buffer and diluted to 1.5 mg/L and mixed with cleaned PCR product to a 6:1 w/w ratio DNA-CS^{PEG5k}-SWCNT.

### Inoculation of Plants

Healthy, fully developed sorghum sudangrass plants were subject to inoculation of DNA-CS^{PEG5k}-SWCNT, either through abaxial surface leaf infusion through a needleless syringe (˜5 mL) or stem injection through a needled syringe (˜0.5 mL). Plants were maintained normally for two days prior to tissue collection. A total of 18 sorghum plants were inoculated with sorghum chloroplast targeting constructs. Six plants were separately inoculated with PL1 construct, PL4 construct, and PL1+PL4 construct, respectively, and three plants were grown without inoculation.

To evaluate plant endogenous DNAse activity, 20 μL of cattle genomic DNA (2.5 ng/μL) was separately injected into stems of three untreated sorghum plants, which were subsequently allowed to grow normally for one day, two days, and one week, respectively, prior to total DNA extraction.

### Tissue Collection and Nucleic Acid Preparations

Two-day post-inoculation, sorghum tissues were sectioned off of living plants and immediately submerged in adequate volumes of RNAlater (ThermoFisher) and subsequently frozen until nucleic acid extraction.

Total DNA was extracted using the MagBind Plant DNA Plus kit following manufacturer's instructions, and total RNA was extracted using the MagBind Total RNA kit following manufacturer's instructions. A portion of total RNAs were synthesized into cDNA using Onescript Reverse Transcriptase kit (ThermoFisher) using manufacturer's instructions. Oligo dT primers used in the PCR reactions are shown in Tables 2 and 3.

TABLE 2
Primer sets used Sybr-based PCR reactions to
detect immunogenic leukotoxin subunit coding
DNA and sorghum reference expression gene.

| | Forward primer | Reverse primer |
|---|---|---|
| Target | 5′-3′ | 5′-3′ |
| PL1 | GAT GGG ATT ATC AAC | CCG AGC TTA AGA AAT |
| | GGA ATT CG | ATA AAT TTC CTC C |
| PL4 | GTA GCA GTT AAT AAA | GAT TTG CTT TTT ACC |
| | ATT ACA CAA AAT ACT | AAA GCA TTT CG |
| | TC | |
| Sorghum | AAC CCG CAA AAC CCC | TAC AGG TCG GGC TCA |
| PP2a | AGA CTA | TGG AAC |

TABLE 3
Primer sets used Taqman-based PCR re-
actions to detect immunogenic leukotoxin
subunit coding DNA and sorghum and
millet reference expression gene.

| | Forward | Reverse | |
|---|---|---|---|
| | primer | primer | Probe |
| Target | 5′-3′ | 5′-3′ | 5′-3′ |
| PL1 | GTT TTA | CCA TTG | CTA TAT |
| | ATA GAT | ACA AAG | TTT GGG |
| | TTG CTT | TTA AAA | GAA AAG |
| | TAA CAG | AGA TTA | AAT AGT |
| | AAA ATA | TTT ACC | ACG G |
| | ATA TAG C | | |
| PL4 | GGA TCT | ACT TTA | CAG TGA |
| | ACA AAA | TCT ACT | TTG CTA |
| | GCA TAT | TGC CCT | AAG AAG |
| | GTA AAA | TGA GTA | AAA CAG |
| | GAT TC | G | AT |
| Sorghum | AAC CCG | TAC AGG | FAM-CCT |
| PP2a | CAA AAC | TCG GGC | TAA CTT |
| | CCC AGA | TCA TGG | ACT GGT |
| | CTA | AAC | GTT GAT |
| | | | GCT CCT |
| | | | CTC- |
| | | | BHQ1 |
| Millet | TGA GAG | AAG AGC | FAM-CTT |
| PP2a | CAG ACA | TGT GAG | CTA TGA |
| | AAT CAC | AGG CAA | TGA ATG |
| | TCA A | ATA A | CTT AAG |
| | | | AAA ATA |
| | | | TGG- |
| | | | BHQ1 |

TABLE 4
Primer sets used for PCR reaction to confirm
insertion of transgenic construct into the
sorghum and millet chloroplast genome.
and millet chloroplasts.

| | Forward | Reverse |
|---|---|---|
| | primer | primer |
| Target | 5′-3′ | 5′-3′ |
| Sorg | GGT AGC TAT | CAGGAAACAGC |
| PL1PL4 | TCT GAA TTC | TATGACCACGT |
| left | TCT TAT TTC | TACTTTTGATG |
| insert | TTG | CCGCT |
| Sorg | TGTAAAACGAC | GTT TGG TAA |
| PL1PL4 | GGCCAGTCAA | TGG TTC TCT |
| right | AGACCCCAACG | ATG CTC |
| insert | AGAAGC | ATT ATT TTC |
| Millet | GCT AGG TGA | TGT TAA AGC |
| PL1PL4 | ACG GGA AAA | AAA TCT ATT |
| left | TAC G | AAA ACT G |
| insert | | |

### Realtime PCR

Nucleic acid preparations were used as templates in realtime PCR reactions using primer sets detailed in Tables 2 and 3. Serine/threonine-Protein Phosphatase (PP2A, Genbank accession XM_002453490) was used as the calibrator gene for sorghum studies given its demonstrated stable expression levels under stress conditions (see Reddy et al., 2016). Sybr-based Realtime PCR reactions of cDNAs were carried out in a BioRad CFX384 (BioRad Laboratories) using PowerUp SYBR Green Master Mix (Applied Biosystems, Massachusetts, U.S.A.), 10 μM of forward and reverse primers, and 1 μL of template (either DNA, RNA, cDNA, or water), in Armadillo PCR plates (ThermoFisher) with Absolute qPCR optical tape seals (ThermoFisher), and analyzed with BioRad CFX Manager Software v3.1 (BioRad) using linear regression to determine quantitation cycle (Cq). Taqman-based Realtime PCR reactions of DNA and cDNA were carried out in a BioRad CFX384 (BioRad Laboratories) using 0.4 U of Accustart II Taq polymerase (Quantabio, New Jersey, U.S.A.), 10 μM of forward and reverse primers, 0.4 μM of gene-specific probe, and 1 μL of template (either DNA, RNA, cDNA, or water), in Armadillo PCR plates (ThermoFisher) with Absolute qPCR optical tape seals (ThermoFisher), and analyzed with BioRad CFX Manager Software v3.1 (BioRad) using linear regression to determine quantitation cycle (Cq).

### Sequencing

PCR products were sequenced directly using the original reactions' gene-specific primers with BigDye Terminator v1.1 Cycle Sequencing Kit (Applied Biosystems, Foster City, Calif., USA) using an Applied Biosystems 3500xL Genetic Analyzer and POP-7 polymer protocol (Applied Biosystems User Guide-4337036).

### Results

### Verification of Constructs

Plasmids containing chloroplast transformation vectors arrived as dry DNA, which were subsequently reconstituted in TE to 5 pg/μL working stocks to be used as templates to PCR amplify expression vectors. Size fractionated PCR products show that amplicons are of appropriate sizes (see FIG. 5), confirming that the samples contained the expression vector.

Evaluation of RNA Preparations and cDNA Synthesis

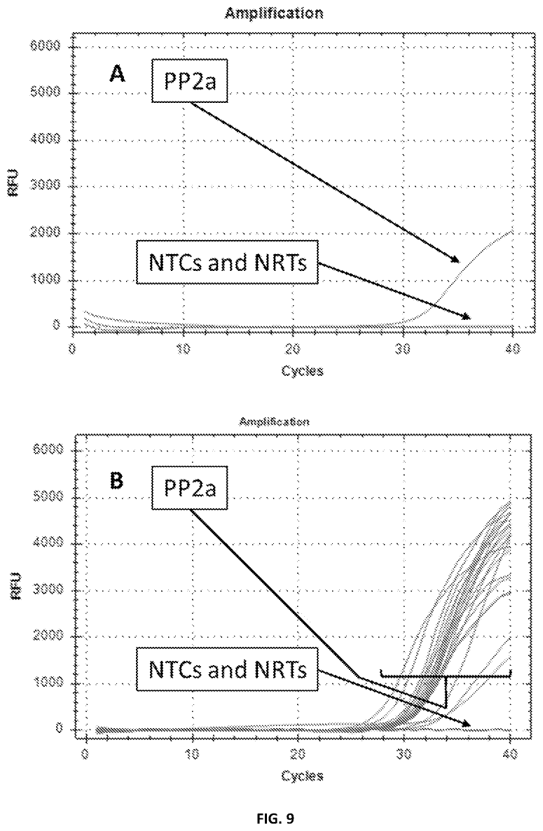

To ensure appropriate handling of total RNA and faithful synthesis of cDNAs from expressed transcripts, PCR assays targeting PP2a, a gene known to be stably expressed in above-ground tissues of millet (Saha and Blumwald, 2014) and sorghum (Reddy et al., 2016) was conducted on cDNAs prepared from both uninoculated and inoculated millet (FIG. 9A) and sorghum (FIG. 9B) plants. Results showed that RNAs were suitably handled and cDNAs were properly synthesized from all plants used in this study.

### Construct Persistence in Living Plant Tissues

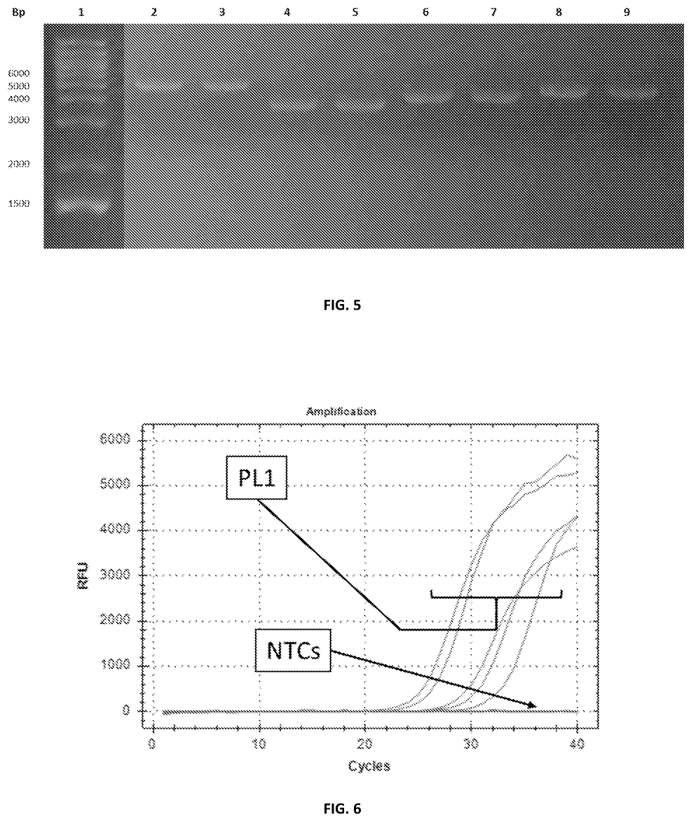

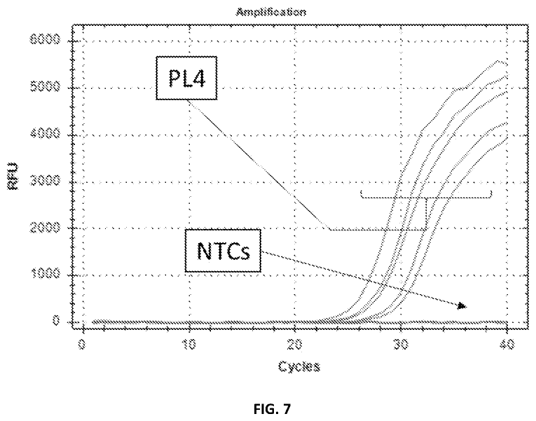

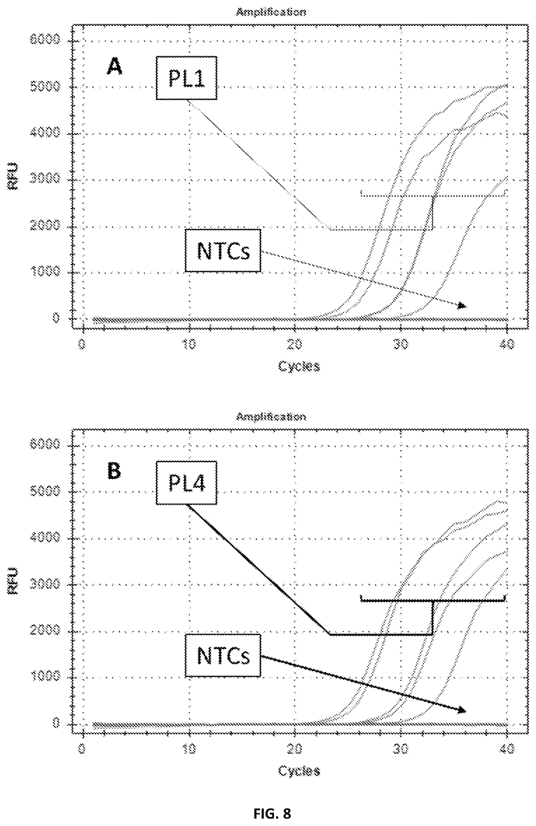

To assess whether expression vector DNA is present and persists in inoculated plant tissues, indicating successful chloroplast transformation, a series of PCR reactions on DNA extracts of uninoculated and inoculated plants were performed. DNAs from all inoculated plants were tested for PL1 and PL4 expression constructs two days after they were inoculated. Results of each test are shown in FIGS. 6-8. These results confirm the presence of PL1 (FIG. 6), PL4 (FIG. 7), and PL1+PL4 (FIG. 8) in all five of the transformed sorghum plants that were tested.

### Evidence for Expression and Chloroplast Chromosome Integration of Immunogenic Leukotoxin A Subunits

To gather evidence that constructs were effectively shuttled to sorghum chloroplasts, successfully recombined with the plastid genome through homologous recombination, and expressed as leukotoxin mRNAs, a series PCRs were conducted using primers from native sorghum chloroplast region, and PL1 and PL4 assays, with DNA and cDNA templates where applicable.

The PCR reactions were performed as described below in a BioRAD CFX96 Optical Thermocycler (BioRad):

| Step | Number of cycles | Temperature (° C.) | Duration | |
|---|---|---|---|---|
| 1 | 1 | 98 | 1 | minute |
| 2 | 30 | 98 | 15 | seconds |
| | | 58 | 30 | seconds |
| | | 72 | 2 | minutes |
| 3 | 1 | 72 | 2 | minutes |

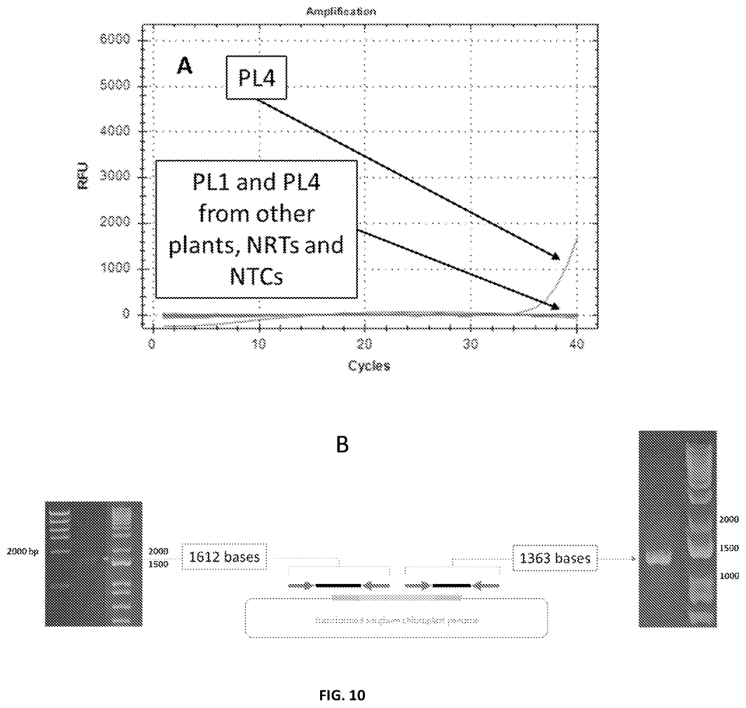

Results showed that one sorghum plant, inoculated with the PL1+PL4 construct, showed detectable levels of transcriptional expression (FIG. 10A). Furthermore, a PCR targeting DNA templates from this PL1+PL4 inoculated sorghum plant with primers positioned outside the left flank of the construct and inside the insert (the PL1 sequence, specifically) resulted in a product of expected size (1612 bases, FIG. 10A). Additionally, a product of expected size (1363 bases) was generated using primers positioned inside the insert (the MTurquoise sequence, specifically) and outside the right flank of the construct (FIG. 10B). This indicates the presence of a continuous template stretching from the native sorghum chloroplast through to F. necrophorum antigen, i.e., successful recombination occurred between the transformation vector and sorghum chloroplast genome. Confirmation by cDNA sequencing showed identical matching of 50 consecutive bases with this PCR product and the corresponding PL4 sequence (FIG. 11).

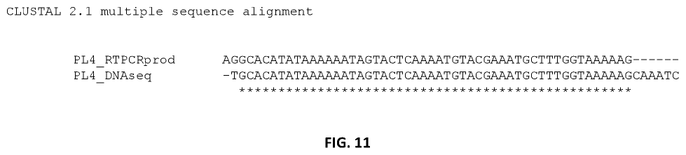

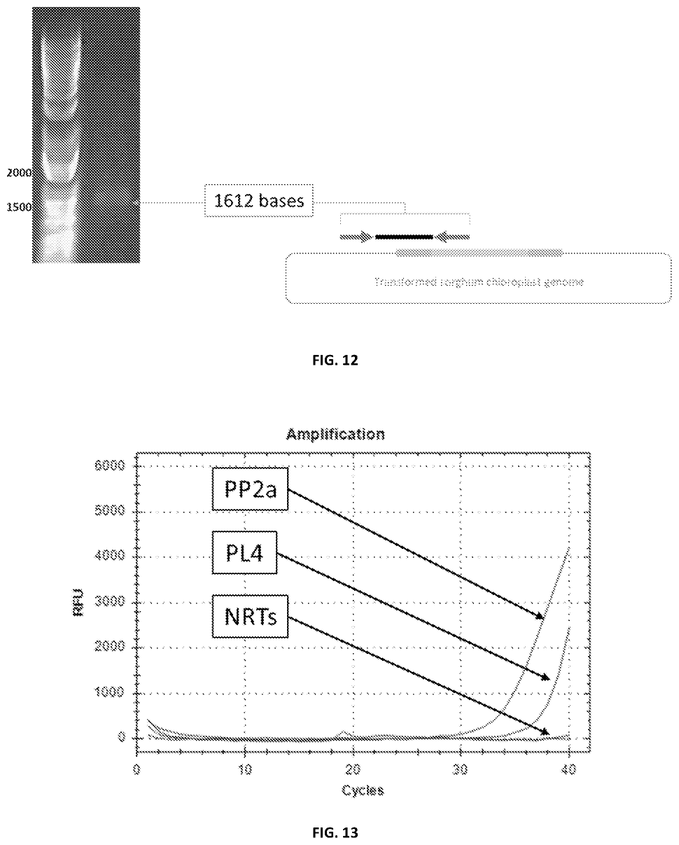

Subsequent efforts to develop mature sorghum plants expressing immunogenic leukotoxin subunits were inoculated with functionalized nanotubes (as described herein) conjugated to PL1+PL4 chloroplast transgenic constructs in a similar manner as described above. Results of a PCR to validate insertion of the transgenic construct into the sorghum chloroplast (i.e. transformation) showed a band of expected size (1612 bases, FIG. 12), and subsequent RT-qPCR results from mRNAs derived from that plant data showed transgenic expression of the PL4 subunit, along with PP2a reference gene and relevant controls (FIG. 13).

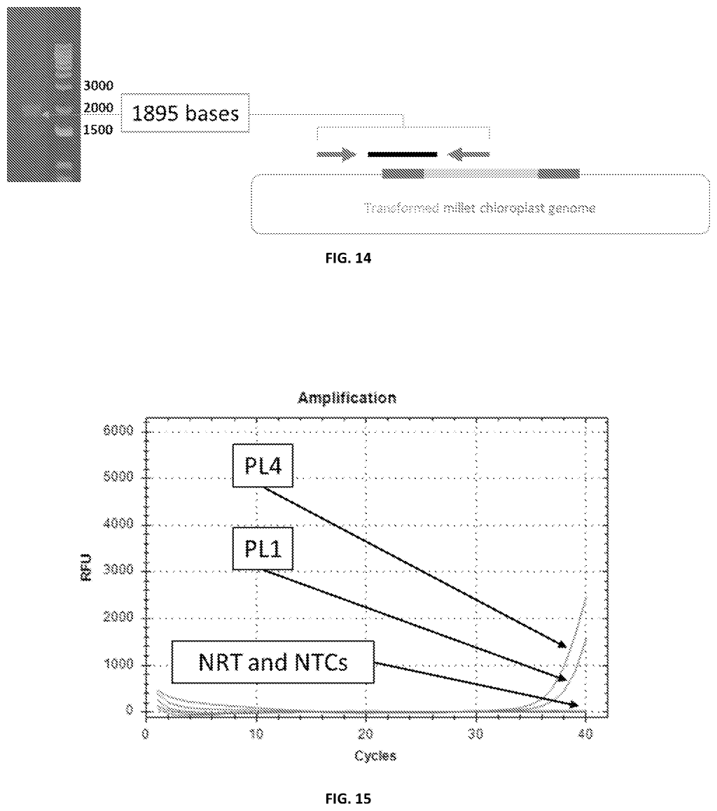

To confirm that constructs were effectively shuttled to millet chloroplasts and successfully recombined with the plastid genome through homologous recombination, PCRs targeting DNA templates from PL1+PL4 inoculated millet plant with primers positioned outside the left flank of the construct and inside the insert (the PL1 sequence, specifically). Results show a band of expected size (1895 bases, FIG. 14), indicating the presence of a continuous template stretching from the native millet chloroplast through to F. necrophorum antigen.

Subsequent RT-qPCRs of this inoculated millet plant shows amplification of PL1 and PL4 cDNAs (FIG. 15), providing evidence that transgenic construct DNA is inducing the millet plant to express the desired F. necrophorum mRNAs.

Evidence for Construct Presence Three Months after Inoculation

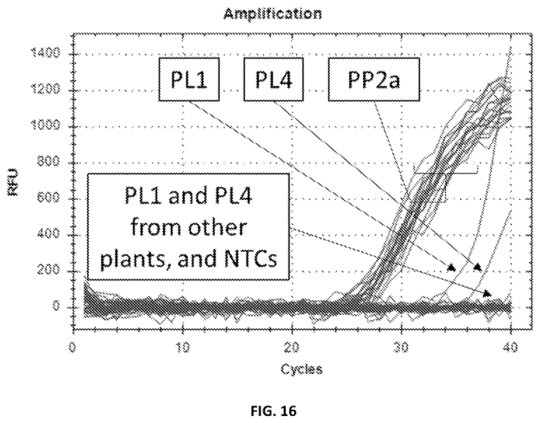

Since nucleic acid extraction involves sacrificing the sampled plant, a series of sorghum plants that were inoculated and set aside for several months such that they could be evaluated as to whether or not construct DNA persisted within the host plants' milieu. To assess this possibility, three month post-inoculated plant tissues above the stem injection sites (where inoculum potentially traveled and where meristematic tissue was presumed to be located) were harvested, had their nucleic acids extracted (RNA and DNA, separately), and prepared to be used as templates in PCR reactions with PL1 and PL4 assays. Plants that survived inoculation and tissue harvesting in the preceding months were also tested in this manner to see if additional evidence of construct persistence could be gathered. The results of all such experiments are shown in FIG. 16. Notably, one plant that was inoculated with a PL1+PL4 construct and not previously tested, showed evidence of both PL1 and PL4 targets. None of the above plants showed mRNA expression of PL1 and PL4 after RNA extractions and cDNA synthesis (results not shown).

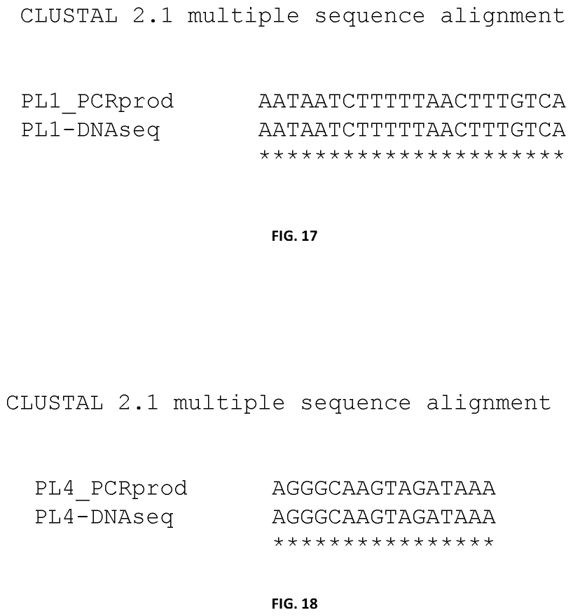

To further authenticate the identity of the PL1 and PL4 amplifications, the sequence of these respective PCR amplicons in FIG. 16 were aligned with their expected sequences (FIGS. 17 and 18). These alignments show 22-bases of identical sequence in the PL1 PCR product (PL1 PCRprod) aligned with the known PL1 sequence (PL1-DNAseq; FIGS. 17), and 16-bases of identical sequence in the PL4 PCR product (PL4_PCRprod) aligned with the known PL4 sequence (PL4-DNAseq; FIG. 18).

### Discussion

The results presented here show evidence that successful transformation and expression events occurred in sorghum seedlings. Such a finding confirms the design of the chloroplast transformation vector were, at least to result in chloroplast chromosome integration and ectopic expression in the host species. This result is the first known attempt and successful transformation of the sorghum chloroplast genome.

The results presented here also show evidence the successful transformation of millet chloroplast with the millet transgenic construct. The selection of genetic elements of the constructs disclosed herein, such as plastid genome targets for homologous recombination and the use of tobacco and phage regulatory elements, along with associated actions with nanotubes (functionalization, DNA conjugation, inoculation, etc.) have no established precedents in this field of research and represent a unique combination.

The amplifications shown in FIG. 10 panel (A) and FIG. 10 panel (B) confirm that the choices made regarding selection of sorghum flanking regions and cassette expressive components (such as promoters) were sufficient to induce homologous recombination and elicit mRNA expression from the cassette, respectively, while FIG. 14 confirm that choices regarding selection of flanking regions were sufficient to induce homologous recombination in the millet chloroplast genome

Further, the plant that showed expression of the PL4 subunit had been inoculated with PL1+PL4 construct, suggesting that PL1 is potentially being expressed as well, yet at a level below detection limit of the methods used in this example.

Another encouraging result is that targets within the DNA construct physically persisted in plants, as targets were observed in all plants two days post-inoculation (FIGS. 6-8), and one plant showed target amplification three months after inoculation (FIG. 16). Native plant DNAses are particularly induced in response to mechanical injury and leaf infiltration (Mittler and Lam, 1996). Since mechanical injury and leaf infiltration both occurred during injection and infusion inoculations, it supports that inoculum DNA reached their intended chloroplast destinations and escaped digestion by apoplastically released DNAses. To further explore this possibility, we injected sorghum plants with cattle genomic DNA in the same manner as the construct injections and tested whether these DNAs were observable over subsequent days, finding no evidence of cattle DNA at the injection site or the leaf tissues above as early as 24 hours post injection (data not shown).

The results presented here provide evidence that sorghum chloroplast-targeting DNA constructs coding for Fusobacterium leukotoxin immunogenic subunits were successfully introduced into plant tissues, where they evaded DNAse digestion for at least two days (and in one case over three months), and one such plant produced transcripts from the inoculant construct two days post-inoculation. Furthermore, plants retained detectible copies of Fusobacterium construct DNA over three months after injection, an observation that suggests that the construct successfully integrated and was retained in the sorghum chloroplast genome.

### Example 6: Administration

Once sufficient quantities of antigen (PL1, PL4, or PL1+PL4) are synthesized by the sorghum and millet plants transformed with constructs 1-4, transformed plants will be diluted into an immunogenic composition with non-transformed sorghum and/or millet to various concentrations of antigen, and fed to at least 5 cattle for each concentration. Cattle will be monitored for differences in behavior and symptoms, looking for any obvious adverse reactions. Ability of the immunogenic composition to elicit an immune response to the PL1 and PL4 immunogenic fragments will be determined by periodic blood serum samples collected for quantification of PL1/PL4 antibodies. Methods for measuring PL1 and PL4 antibodies in serum include:
1. Coat ELISA plates in PL1 and PL4 antigen (diluted in carbonate buffer):
2. Wash plate in PBST;
3. Block plate with 2% fat-free dry milk in carbonate buffer for 60 minutes;
4. Apply cattle blood serum (1:50 in carbonate buffer) to the wells;
5. Incubate the wells at 37° C. for 60 minutes;
6. Wash plate in PBST;
7. Add 1:5000 secondary antibody conjugated with horseradish peroxidase (Zymed, San Francisco, Calif., USA);
8. Wash plate in PBST;
9. Add 0.3 mg/L 2-20 Azino-bis-3 etilbenztiasoline-6-sulphuric acid (ABTS; Sigma, Mo., USA) and 0.1 M citric acid, pH 4.35;
10. Using a Multiskan Ascent (Thermo Scientific, Massachusetts, USA) microplate reader, record the optical density at 405 nm; and
11. Expressed PL1 and PL4 will be quantified by comparing OD405 in 100 mg of total protein to OD405 of standard curve.

Serum from unvaccinated cattle will be used as a negative control in ELISA. The immunogenic responses of cattle fed the immunogenic composition at various doses over time will be calibrated against a standard curve. To determine cattle serum results, we will convert OD values to ELISA units using the following formula:

(mean net sample OD−mean net negative-control OD)÷(mean net positive-control OD−mean net negative-control OD)×100

Finally, carcass information from these animals will be compared (to a control animal and among the various treatment groups), particularly to examine the potential reduction in Fusobacterium-related abscesses, and determine optimal dosing of the immunogenic composition, and the effects of using one or more ltkA fragments.

### Example 7: Development of a Treatment in Cattle for Liver Abscess

Previous examples have demonstrated the insertion of bacterial DNA constructs containing immunodominant regions of leukotoxin (PL1 and/or PL4) into the chloroplast of sorghum and millet plants and expression of the immunodominant regions. The example below utilizes these transformed sorghum and millet plants expressing bacterial antigens for in-feed vaccination of livestock. The examples below demonstrate methods of developing/using a liver abscess challenge model; methods of administering chloroplast-transformed plants to cattle; measuring adverse events and using an assay to measure seroconversion of cattle (antibody titer) to the plant-based antigen (i.e., PL1 and/or PL4 of leukotoxin), particular dosing regimens of administering chloroplast-transformed plants to cattle, and determining the efficacy of feeding chloroplast-transformed plants in preventing liver abscess development in cattle.

Liver Abscess Challenge Model

In order to provide a reliable measure of effectiveness of provided therapies, a liver abscess induction model is produced by injecting F. necrophorum into the hepatic portal vein or caudal vein through an ultrasound-guided percutaneous catheter in order to approximate natural development of liver abcess in cattle after F. necrophorum exposure (Lechtenberg et al., 1989 and 1991). One of skill in the art will appreciate that other forms of administration may achieve the same goal (e.g., distribution of the antigen into the circulation).

The challenge model will be capable of repeatable induction of a moderate prevalence and severity of liver abscesses in young calves. Ability to perform such a challenge will allow the subsequent testing of liver abscess preventions (vaccines) or treatments in a controlled setting while requiring a relatively small amount of test product.

Fusobacterium necrophorum culture. A virulent, high leukotoxin-producing strain of F. necrophorum subspecies necrophorum isolated from a liver abscess of beef cattle will be grown on VersaTREK REDOX 2 (Trek Diagnostic Systems, OH) anaerobically at 37° C., and we expect that after 12 h of growth, cells will be harvest at approximately 1.0×10¹² CFU/ml (NASEM 2016). Serial dilution plating and CFU counting will be performed using the Fusobacterium Selective Agar (Anaerobe Systems, CA) at anaerobic conditions at 37° C. for 24 to 48 h.

In this example, there will be two experimental phases:

Treatments Phase I. Twenty bull calves will be randomly assigned to four experimental treatments (n=5 calves per treatment).
CONPV: control calves will be inoculated with sterile saline
FUSOPV8: calves will be inoculated with 10 mL of sterile saline inoculated with 2×10⁷ CFU/ml of F. necrophorum. Total inoculated 2×10⁸ CFU of F. necrophorum.
FUSOPV9: calves will be inoculated with 10 mL of sterile saline inoculated with 2×10⁸ CFU/ml of F. necrophorum. Total inoculated 2×10⁹ CFU of F. necrophorum.
FUSOPV10: calves will be inoculated with 10 mL sterile saline inoculated with 2×10⁹ CFU/ml of F. necrophorum. Total inoculated 2×10¹⁰ CFU of F. necrophorum.

For each treatment, inoculation will be intraportally using an ultrasound-guided percutaneuous catherterization technique.

Treatments Phase II. Twenty bull calves will be randomly assigned to four experimental treatments (n=5 calves per treatment).
CONIV: control calves will be inoculated with sterile saline
FUSOIV9: calves will be inoculated with 10 mL sterile saline inoculated with 2×10⁸ CFU/ml of F. necrophorum. Total inoculated 2×10⁹ CFU of F. necrophorum.
FUSOIV10: calves will be inoculated with 10 mL sterile saline inoculated with 2×10⁹ CFU/ml of F. necrophorum. Total inoculated 2×10¹⁰ CFU of F. necrophorum.
FUSOIV11: calves will be inoculated with 10 mL sterile saline inoculated with 2×10¹⁰ CFU/ml of F. necrophorum. Total inoculated 2×10¹¹ CFU of F. necrophorum.

Doses evaluated are based on a previously established murine model, and adjusted for differences in body mass of mice and calves (see Nagaoka, K. et al. 2013). Doses will be administered through jugular infusion.

Data Collection. In both phases ultrasonography of the liver will be performed prior to inoculation and at 1, 2, 5, and 7 days after inoculation to evaluate the progression of liver abscesses. At the same time points, blood will be collected and analyzed for blood leukocyte counts and concentrations of the plasma acute-phase proteins haptoglobin and serum-amyloid A. These results will demonstrate both the development of liver abscess and measure the inflammatory response associated with F. necrophorum challenge. Throughout the study, calves will be monitored for adverse events and have rectal temperature recorded.

At the end of the study, calves will be euthanized and necropsy performed to confirm the presence and severity of liver abscesses and other pathology.

### Dosing Regimens

In order to calibrate provided exemplary therapies, a dose finding study is performed. The study will include determining the effective feeding level of immunogenic plants for producing an antibody response. Various dosing regimens will be tested that include continuous- and “pulse-fed” administration of the immunogenic plants to cattle, and the antibody response of the cattle will be observed as well as the monitoring for any adverse events.

Methods:

Animals. Ruminating calves (BW>300 lb.) will be utilized in this experiment. Calves will be housed in individual pens for the duration of the study, provided ad libitum access to water and fed a mixed ration formulated to meet or exceed nutrient requirements (NASAM 2016).

Treatments. During a 28-day experiment, immunogenic plants will be included in the ration as a portion of the total feed ration based on the antigen concentration as a percentage of total soluble protein in the immunogenic plant. A ration containing transgenic peanut expressing an active protein to provide 0.5% of total soluble protein will be used. Dose range will be targeted to provide 0.5%, 1%, and 2% of total soluble protein if possible.
Seven treatments (n=5 ruminating calves per treatment) are expected to be used (including in a 3 doses×2 timelines for administration+1 control).
   Negative control: no immunogenic plant fed.
   LOCON: Low dose (0.5%) immunogenic plant fed continuously for 28 days.
   MEDCON: Medium dose (1%) immunogenic plant fed continuously for 28 days.
   HICON: High dose (2%) immunogenic plant fed continuously for 28 days.
   LOPUL: Low dose (0.5%) immunogenic plant fed in 3 one-day pulses on day 0, 7, and 14.
   LOPUL: Medium dose (1%) immunogenic plant fed in 3 one-day pulses on day 0, 7, and 14.
   HIPUL: High dose (2%) immunogenic plant fed in 3 one-day pulses on day 0, 7, and 14.

Experimental Diets and Feeding. Immunogenic plants will be harvested as hay, dried (air dried 85-90% dry matter), and ground through a 2-3″ screen. Hay will be mixed as a portion of a growing ration (35-45% total roughage concentration expected) using mixing equipment appropriate for the batch size. Cattle will be fed daily, ad libitum, experimental rations throughout the experiment.

Measurements and Data Collection. Calves will be evaluated daily throughout the experiment for adverse events and signs of illness. On days 0, 7, 14, 21, and 28, blood will be collected for acute-phase protein and cytokine determination (haptoglobin, fibrinogen, interleukin-6, and tumor necrosis factor-α).

An antibody titer assay will be used to detect the presence of antibodies associated with the antigen utilized in the immunogenic plant. The antibody titer assay will include antibodies specific to the truncated regions of F. necrophorum included in the immunogenic plant and an assay to quantify antibody titers to leukotoxin A in its entirety. PL1 and PL4 antigens obtained from a bacterial model will be used to obtain and antigen-specific antibody.

Acute-phase protein data will be used to determine inflammatory response associated with feeding immunogenic plants and antibody titers will measure memory immune response. Antibody concentration on day 28 will be used to select dose for subsequent experiments.

Necropsy. Any animal that dies during the experiment will be necropsied. At the end of the experiment, cattle will be euthanized and submitted for full necropsy. Livers will be observed for the presence and severity of liver abscesses, and other pathogenesis will be noted. Specific tissues could be submitted for histopathology.

### Extended Immunity

The various dosing regions (e.g., continuous and pulse-fed regimens) will be evaluated for the ability to elicit extended immune protection and for safety over extended periods of administration. To do this, cattle will be feed according to the dosing regimens evaluated above, for periods of time of up to 16 weeks. Antibody response will be measured throughout the feeding period and cattle will be monitored for adverse events.

Methods:

Animals. Ruminating calves (BW=700 lb.) will be utilized in this experiment. Calves will be housed in individual pens for the duration of the study, provided ad libitum access to water and fed a mixed ration formulated to meet or exceed nutrient requirements.

Treatments. Five treatments groups (n=10 ruminating calves per treatment) will be fed over 16 weeks. Antibody titers and acute phase protein responses will be measured weekly.

### Treatment Groups:

1. CON: Negative control, no immunogenic plant fed
2. SHORT: Short-term continuous feeding of the immunogenic plant for 7 days
3. LONG: Continuous feeding of the immunogenic plant for 16 sequential weeks
4. SHORT PULSE: Short-term pulse of dosing of the immunogenic plant on d 0, 7, and 14.
5. WEEKLY: feeding the immunogenic plant once per week for 16 sequential weeks

Experimental Diets and Feeding. Immunogenic plants will be harvested as hay, dried (air dried 85-90% dry matter), and ground through a 2-3″ screen. Hay will be mixed as a portion of a growing ration (35-45% total roughage concentration expected) using mixing equipment appropriate for the batch size. Cattle will be fed daily, ad libitum, experimental rations throughout the experiment.

Measurements and Data Collection. Calves will be evaluated daily throughout the experiment for adverse events and signs of illness. On days 0, 7, 14, 21, and 28, 56, 84, and 112, blood will be collected for acute-phase protein and cytokine determination (haptoglobin, fibrinogen, interleukin-6, and tumor necrosis factor-a) measurement of antibody titers (Experiment 2 above). Acute-phase protein data will be used to determine inflammatory response associated with feeding immunogenic plants and antibody titers will measure memory immune response.

Necropsy. Any animal that dies during the experiment will be necropsied.

Harvest. At the end of the experiment, cattle will be harvested at a commercial abattoir where livers will be scored for the presence and severity of liver abscesses.

### Liver Abscess Challenge Model to Determine Protection

In order to evaluate the ability of the immunogenic plants to reduce abscess severity in cattle, the liver abscess challenge model will be used to develop liver abscess in cattle, which will be subsequently fed an immunogenic plant as described herein, including in the above Examples. Healthy cattle will be examined to determine if feeding of the immunogenic plant provides protection from abscess development. Cattle in both cases will be monitored throughout the treatment (i.e., during administration of the immunogenic plant).

Methods:

Various dosing regimens tested will be used in this study. Ruminating calves (at least 5 per treatment) will be used, and a negative control treatment (calves not fed the immunogenic plant) will be included. After feeding the experimental diets for at least 28 days, calves will be challenged using the F. necrophorum model described.

During the experiment, ultrasonography of the liver will be used to evaluate the progression of liver abscesses and performed prior to inoculation and at 1, 2, 5, and 7 days after inoculation. At the same time points, blood will be collected and analyzed for blood leukocyte counts and concentrations of plasma acute-phase proteins haptoglobin and serum-amyloid A. Serum analysis for blood leukocyte counts and concentrations of plasma acute-phase proteins haptoglobin and serum-amyloid A will measure the inflammatory response associated with F. necrophorum challenge. Throughout the study, calves will be monitored for adverse events and have rectal temperature recorded.

At the end of the study, calves will be euthanized and necropsy performed to confirm the presence and severity of liver abscesses and other pathologies.

### Immune Protection and Performance in Production Settings

Production settings involve specific conditions (i.e., size of pen, number of animals, age of animals, etc.). In order to test the safety and efficacy of immunogenic plants in a production setting, these settings will be modeled and finishing steers will be the fed immunogenic plant. Finishing steers will be monitored for adverse events. Additionally, finishing performance and carcass characteristics of cattle fed immunogenic plants will be measured. Through this monitoring, effects of immunogenic chloroplast-transformed plants on liver abscess prevalence and severity will be determined.

Methods:

Animals. Beef cattle, steers or heifers will be used in this study. Number will be determined based on chosen treatment design. Each treatment will have a minimum of 10 pen replications (n=70 steers per pen). Cattle will be housed in open, dirt-floored pens (60 ft wide×172 ft deep) of welded steel pipe construction. Each pen will have a continuous concrete feed bunk and share a float-controlled water tank with an adjacent pen.

Treatments. Treatments will include a negative control in which cattle are fed a no immunogenic chloroplast-transformed plants or tylosin, and 2 to 3 treatments containing immunogenic plants. Dosing regimens based on initial experiments will be utilized. Experimental diets will be formulated to meet or exceed nutrient requirements. Formulation between the treatment groups will be identical except for the inclusion of immunogenic plants for necessary treatment rations. The control diet will be formulated to contain non-transformed plant at equal concentration.

A generalized randomized block design or randomized complete block design will be used with the pens serving as the experimental unit (n=60 to 70 head per pen and a minimum of 10 pen replications per treatment). A pre-prepared chute-order randomization schedule created via a Microsoft Excel random number generator will assign each study candidate to a pen and each pen to a dietary treatment. Cattle determined to be±two standard deviations from the average pay weight (standard deviation determined from facility records) at the time of randomization to treatments and any animal or otherwise unsuitable for study (ill or injured) will be excluded from the trial. Each study candidate will be identified with duplicate, uniquely numbered individual identification tags. Cattle within a block will be housed in sequential pens within the same alley.

Feed Milling. Experimental diets will be prepared in the on-site feedmill, which is equipped for steam-flaking grains, has a computerized batching system and micro-ingredient weigh machine (Micro Beef Technologies, Amarillo, Tex.), and a horizontal paddle mixer. Immunogenic feed ingredient will be added directly to the feed truck either via the micro-ingredient machine or directly through the feed mill (depending on the amount that will need to be added). Mixed feed is conveyed to overhead bins where it is held until dispensed into trucks for delivery to the pens. Batch size will be approximately 8000 lb.

Feed will be delivered to the pens using trucks fitted with mixer boxes (Roto-Mix,Dodge City, Kans.) mounted on load cells and equipped with a GPS unit and computerized system (Read-N-Feed, Micro Beef Technologies) for scheduling, routing and recording feed deliveries. Daily feed records will maintained in a database.

Data Collection. Data to be collected:
Group weights collected on a pen scale on study day 0 (first day of feeding treatment diets) prior to feeding. Platform scales are tested and certified by an independent company every 6 months, and zeroed between each draft of cattle.
Final weight collected on a pen scale on the day of shipment for harvest. Weight will be multiplied by 0.96 to account for gastrointestinal fill.
Daily feed offered and daily ration dry matter to calculated DMI.
Necropsy reports from dead animals and records for removal or treatment of cattle that may become sick or injured.
Carcass data—harvest dates will be coordinated with Beef Carcass Research Center

(BCRC), who will conduct tag transfer at the plant and collect HCW and liver score. Individual animal ID, recorded by BCRC, will then be correlated with plant data for USDA assigned Quality and Yield Grades, and camera data (LM area, FT, and REA).

### REFERENCES

Adem M, Beyene D, Feyissa T. Recent achievements obtained by chloroplast transformation. Plant Methods. 2017;13:30. Published 2017 Apr. 19. doi:10.1186/s13007-017-0179-1.
Am J Vet Res. 1982 Sep;43(9):1580-6. Studies of Fusobacterium necrophorum from bovine hepatic abscesses: biotypes, quantitation, virulence, and antibiotic susceptibility. Berg J N, Scanlan C M.
Bevis B J, Glick B S. Rapidly maturing variants of the Discosoma red fluorescent protein (DsRed). Nat Biotechnol. 2002 Jan; 20(1):83-7
Brown H, Bing R F, Grueter H P, McAskill J W, Cooley C O, Rathmacher R P. Tylosin and chloretetracycline for the prevention of liver abscesses, improved weight gains and feed efficiency in feedlot cattle. J Anim Sci. 1975 Feb; 40(2):207-13.
Brown H, Elliston N G, McAskill J W, Muenster O A, Tonkinson L V. Tylosin phosphate (TP) and tylosin urea adduct (TUA) for the prevention of liver abscesses, improved weight gains and feed efficiency in feedlot cattle. J Anim Sci. 1973 Nov; 37(5):1085-91.
Cardi T, Lenzi P, Maliga P. Chloroplasts as expression platforms for plant-produced vaccines. Expert Rev Vaccines 2010; 9:893-911; PMID:20673012; dx.doi.org/10.1586/erv.10.78.
Chen H F, Chang M H, Chiang B L, Jeng S T. Oral immunization of mice using transgenic tomato fruit expressing VP1 protein from enterovirus 71. Vaccine. 2006 Apr 5;24(15):2944-51. Epub 2006 Jan 17.
Checkly, Sylvia L., Janzen, Eugene D., Campbell, John R., and John J. McKinnon. Can Vet J. 2005 Nov; 46(11): 1002-1007.PMCID: PMC1259145PMID: 16363327. Efficacy of vaccination against Fusobacterium necrophorum infection for control of liver abscesses and footrot in feedlot cattle in western Canada.
Demirer G S, Zhang H, Matos J L, Goh N S, Cunningham F J, Sung Y, Chang R, Aditham A J, Chio L, Cho M J Staskawicz B, Landry M P. High aspect ratio nanomaterials enable delivery of functional genetic material without DNA integration in mature plants. Nature Nanotechnology. 2019. Feb 25 (14):456-464.
Goedhart J, von Stetten D, Noirclerc-Savoye M, Lelimousin M, Joosen L, Hink M A, van Weeren L, Gadella T W Jr, Royant A. Structure-guided evolution of cyan fluorescent proteins towards a quantum yield of 93%. Nat Commun. 2012 Mar 20;3:751. doi: 10.1038/ncomms1738.
Hanson M R, Gray B N, Ahner B A. Chloroplast transformation for engineering of photosynthesis. J Exp Bot. 2013 Jan;64(3):731-42. doi: 10.1093/jxb/ers325. Epub 2012 Nov 16.
Heifetz P B. Genetic engineering of the chloroplast. Biochimie. 2000 Jun-Jul;82(6-7):655-66.
Khandelwal, A., Sita, G. L., & Shaila, M. S. Oral immunization of cattle with hemagglutinin protein of rinderpest virus expressed in transgenic peanut induces specific immune responses. Vaccine 3812, 1-8 (2003).
Kwak S Y1, Lew T T S1, Sweeney C J1, Koman V B1, Wong M H1, Bohmert-Tatarev K2, Snell K D2, Seo J S 3, Chua N H3, Strano M S4. Nat Nanotechnol. 2019 May;14(5):447-455. doi: 10.1038/s41565-019-0375-4. Epub 2019 Feb 25. Chloroplast-selective gene delivery and expression in planta using chitosan-complexed single-walled carbon nanotube carriers.
Lakshmi, P. S., Verma, D., Yang, X., Lloyd, B. and Daniell, H. (2013) Low cost tuberculosis vaccine antigens in capsules: expression in chloroplasts, bio-encapsulation, stability and functional evaluation in vitro. PLoS ONE, 8, e54708.
Lawrence, Ty. www.bovinevetonline.com/article/liver-abscesses-beyond-just-liver-condemnation. Accessed February, 2020.
Lechtenberg, K. F., Nagaraja, T. G., Avery, T. B. & Hartke, G. T. Ultrasound-guided, percutaneous catheterization of the portal vein in cattle. Agri-Practice 10, 41-42 (1989).
Lechtenberg, K. F. & Nagaraja, T. G. Hepatic ultrasonography and blood changes in cattle with experimentally induced hepatic abscesses. Am. J. Vet. Res. 52, 803-809 (1991).
Lu Y, Stegemann S, Agrawal S, Karcher D, Ruf S, Bock R. Horizontal Transfer of a Synthetic Metabolic Pathway between Plant Species. Curr Biol. 2017 Oct 9;27(19):3034-3041.e3. doi: 10.1016/j.cub.2017.08.044. Epub 2017 Sep 21.
Loza-Rubio E, Rojas-Anaya E, López J, Olivera-Flores M T, Gómez-Lim M, Tapia-Pérez G. Induction of a protective immune response to rabies virus in sheep after oral immunization with transgenic maize, expressing the rabies virus glycoprotein. Vaccine. 2012 Aug 10;30(37):5551-6. doi: 10.1016/j.vaccine.2012.06.039. Epub 2012 Jun 27.
McBride K E, Svab Z, Schaaf D J, Hogan P S, Stalker D M, Maliga P. Amplification of a chimeric Bacillus gene in chloroplasts leads to an extraordinary level of an insecticidal protein in tobacco. Biotechnology (N Y). 1995 Apr;13(4):362-5.
Mittler, R., Lam, E. Characterization of nuclease activities and DNA fragmentation induced upon hypersensitive response cell death and mechanical stress. Plant Mol Biol 34, 209-221 (1997). https://doi.org/10.1023/A: 1005868402827
Nagai T, Ibata K, Park E S, Kubota M, Mikoshiba K, Miyawaki A. A variant of yellow fluorescent protein with fast and efficient maturation for cell-biological applications. Nat Biotechnol. 2002 Jan; 20(1):87-90.
Nagaoka, K. et al. Establishment of a new murine model of liver abscess induced by Fusobacterium necrophorum injected into the caudal vein. J. Med. Microbiol. 62, 1755-1759 (2013).
Nagaraja T G, Sun Y, Wallace N, Kemp K E, Parrott C J. Effects of tylosin on concentrations of Fusobacterium necrophorum and fermentation products in the rumen of cattle fed a high-concentrate diet. Am J Vet Res 1999;60:1061-5.
Narayanan, S. K. a, G. C. Stewart b, M. M. Chengappa a, T. G. Nagaraja a,* Fusobacterium necrophorum: A ruminal bacterium that invades liver to cause abscesses in cattle. S. Tadepalli a,lAnaerobe 15 (2009) 36-43.
Narayanan S K, Nagaraja T G, Chengappa M M, Stewart G C. Leukotoxins of gram-negative bacteria. Vet Microbiol 2002;84:337-56.
Narayanan S K, Stewart G C, Chengappa M M, Nagaraja T G. Fusobacterium necrophorum leukotoxin induces activation and apoptosis of bovine leukocytes. Infect Immun 2002;70:4609-20.
Narayanan S K, Nagaraja T G, Chengappa M M, Stewart G C. Cloning, sequencing, and expression of the leukotoxin gene from Fusobacterium necrophorum. Infect Immun. 2001;69(9):5447-5455. doi:10.1128/iai.69.9.5447-5455.2001.
NASEM. Nutrient Requirements of Beef Cattle: 8^{th} Revised Edition. National Academies of Science Engineering and Medicin. (2016).
Ormö M, Cubitt A B, Kallio K, Gross L A, Tsien R Y, Remington S J. Crystal structure of the Aequorea victoria green fluorescent protein. Science. 1996 Sep 6;273(5280):1392-5.
Palakolanu Sudhakar Reddy*, Dumbala Srinivas Reddy, Kaliamoorthy Sivasakthi, Pooja Bhatnagar-Mathur, Vincent Vadez and Kiran K. Sharma. Frontiers in Plant Science 25 April 2016 volume 7 article 529 Evaluation of Sorghum [Sorghum bicolor (L.)] Reference Genes in Various Tissues and under Abiotic Stress Conditions for Quantitative Real-Time PCR Data Normalization Rigano, M. Manuela,1 Nunzia Scotti2 and Teodoro Cardi. Bioengineered 3:6, 329-333; November/December 2012;© 2012 Landes Bioscience Unsolved problems in plastid transformation.
Rubio-Infante N1, Govea-Alonso D O, Alpuche-Solis Á G, Garcia-Hernández A L, Soria-Guerra R E, Paz-Maldonado L M, Ilhuicatzi-Alvarado D, Varona-Santos J T, Verdín-Terán L, Korban S S, Moreno-Fierros L, Rosales-Mendoza S. A chloroplast-derived C4V3 polypeptide from the human immunodeficiency virus (HIV) is orally immunogenic in mice. Plant Mol Biol. 2012 Mar;78(4-5):337-49. doi: 10.1007/s11103-011-9870-1. Epub 2012 Jan 7.
Ruhlman T1, Verma D, Samson N, Daniell H. Plant Physiol. 2010 Apr;152(4):2088-104. doi: 10.1104/pp.109.152017. Epub 2010 Feb 3. The role of heterologous chloroplast sequence elements in transgene integration and expression.
Rybicky E P. Plants-produced vaccines: promise and reality. Drug Discov Today. 2009;14:16-24.
Saski C, Lee S B, Fjellheim S, Guda C, Jansen R K, Luo H, Tomkins J, Rognli O A, Daniell H, Clarke J L. Complete chloroplast genome sequences of Hordeum vulgare, Sorghum bicolor and Agrostis stolonifera, and comparative analyses with other grass genomes. Theor Appl Genet. 2007 Aug;115(4):571-90. Epub 2007 May 30.
Shahid, Naila, Daniell, Henry. Plant Biotechnology JournalVolume 14, Issue 11 Review Article Open Access. Plant-based oral vaccines against zoonotic and non-zoonotic diseases. 2016.
Sun D B, Wu R, Li G L, Zheng J S, Liu X P, Lin Y C, Guo D H. Identification of three immunodominant regions on leukotoxin protein of Fusobacterium necrophorum. Vet Res Commun. 2009 Oct;33(7):749-55. doi: 10.1007/s11259-009-9223-6.
Wei Z, Liu Y, Lin C, Wang Y, Cai Q, Dong Y, et al. Transformation of alfalfa chloroplasts and expression of green fluorescent protein in a forage crop. Biotechnol Lett 2011; 33:2487-94; PMID:21785988; dx.doi.org/10.1007/s10529-011-0709-2
Wesolowska A, Kozak Ljunggren M, Jedlina L, Basalaj K, Legocki A, Wedrychowicz H, Kesik-Brodacka M. A Preliminary Study of a Lettuce-Based Edible Vaccine Expressing the Cysteine Proteinase of Fasciola hepatica for Fasciolosis Control in Livestock. Front Immunol. 2018 Nov 13;9:2592. doi: 10.3389/fimmu.2018.02592. eCollection 2018.
Yu Q, Lutz K A, Maliga P. Efficient Plastid Transformation in Arabidopsis. Plant Physiol. 2017 Sep;175(1):186-193. doi: 10.1104/pp.17.00857. Epub 2017 Jul 24.
Yukoh Hiei, Yuji Ishida and Toshihiko Komari* Front. Plant Sci., 07 November 2014 | doi.org/10.3389/fpls.2014.00628 Progress of cereal transformation technology mediated by Agrobacterium tumefaciens.
Zou C, Li L, Miki D, Li D, Tang Q, Xiao L, Rajput S, Deng P, Peng L, Jia W, Huang R, Zhang M, Sun Y, Hu J, Fu X, Schnable P, Chang Y, Li F, Zhang H, Feng B, Zhu X, Liu R, Schnable J C, Zhu J K, Zhang H. The genome of broomcorn millet. Nat Commun. 2019 Jan 25;10(1):436. doi: 10.1038/s41467-019-08409-5.

Sequence Listing

| SEQ ID | | |
|---|---|---|
| NO: | Description | Sequence |
| 1 | Left | AGTATTTAGTAACCCGATACAAAATAAATAAAAAGAAAGGCCCTTTTTTCGAAAAA |
| | Flank_{Sorghum} | ATTCGGATTTTTATACACATTAAAATGCTATTTTCGTTCCGAATTATTACCTATTC |
| | | TCTTTCATTATTATGAAATTCTATTGCCATTAGAATATTGATTGATAGACAATTAA |
| | | TAAAAAGAAAACTCTAATAGAAAATGAAACGGTCGACCCAGACATAGACGGTCGAC |
| | | CCGGGTGGATATACCCTATAAAATATAGGCCGTAGCAAGCGTAGTTCAATGTAGCG |
| | | AGCGTAGTTCAGTGGTAAAACATCTCCTTGCCAAGGAGAAGATACGGGTTCGATTC |
| | | CCGTCGCTCGCCAGCTTAATTTAGTAAGGTGCTATGATAAAAAATTCAGTTTAGTT |
| | | GACTACTTAACAACTTCTATTAAATTACTATTTAATATTAAATGAATATGAAATTC |
| | | AGTAGTTGTTAGTCTAGTACTGTACCCCTTCCTATCTTATCCTTCCTTTGCACCCG |
| | | ACTCAAAAAAAAGAGTGCTTCGAGGGCGCAAATTCAACTTTCTAAGAAAGTTCCCT |
| | | AAACCGGGGATTCGCCGAGACAAACAAGAGACAAACGGTTTTGAAAGGGGGATAGG |
| | | CTATGCTTTTCTTTCATTTTTTTTTCTGCCTGCTGAATAAAAAAAGGGTTGGATAT |
| | | AGCCCTCTATCATATATATAGAAATAGAATAGTCCATTTATACGGACTGCTAAGTG |
| | | CGGAGACGGGAATCGAACCCGTGACCTCAAGGTTATGAGCCTCGTGAGCTACCAAA |
| | | CTGCTCTACTCCGCTCTGTAGGGCCGAAAACTGGTGGACGAAAGAAAAAGGTTGAA |
| | | TAGAAGTCTCTACCATGTCTAGACAAACAAATGGAATAGTCTTTTTATACAGAATG |
| | | GAGCGGGTAGCGGGAATCGAACCCGCATCGTTAGCTTGGAAGGCTAGGGGTTATAG |
| | | TCGACGTTGGTTGATTATTTTTGACGTCTCTAATTCAAAACCGAACATGAAATTTT |
| | | GATTTCATTCGGCTCCTTTATGGATATTCTCACCACTTAACATCTATGTCAGCTTT |
| | | TCTGTCTGAATGGAACCAAAGCTCTCTGCTTTCTAGATGATCCTTATAGAGTAGGA |
| | | GATAGAAATTCTCCTAAATATCTATCTAATCTACTTACTTCGTTCCCTAATTTCAT |
| | | TCAAGAGATCCTGAGGAAAAGAGTTGGGTTTCCACCGAGCTGAAACAATATAATAT |
| | | ACTGATGGTTCTAGTAAACCAAAACCATCGTTTTTTAGCTATTGGGCTTCCATTTC |
| | | CTACAAAACAAAAGAAGATTTAGTTACGATTGGAAATAAACTTTTTTGTATC |
| 2 | Prrn_{Nicotiana} | GGTACCCCAAAGCTCCCCCGCCGTCGTTCAATGAGAATGGATAAGAGGCTCGTGGG |
| | | ATTGACGTGAGGGGGCAGGGATGGCTATATTTCTGGGAGCGAACTCCGGGCGAATA |
| | | CGAAGCGCTTGGATACG |
| 3 | Lgene10 T7 | GGGAGACCACAACGGTTTCCCTCTAGAAATAATTTTGTTTAACTTTAAGAAGGAGA |
| | phage | TATACCC |
| 4 | PL1_{Fusobacteria} | ATGAGCGGCATCAAAAGTAACGTTCAGAGGACAAGGAAGAGGATATCAGATTCTAA |
| | | AAAAGTTTTAATGATTTTGGGATTGTTGATTAACACTATGACGGTGAGGGCTAATG |
| | | ATACAATCGCCGCGACTGAGAATTTTGGAACAAAAATAGAAAAAAAGGATAATGTT |
| | | TATGACATTAGTACAAACAAGATTCAAGGGGAGAACGCTTTTAACAGTTTTAATAG |
| | | ATTTGCTTTAACAGAAAATAATATAGCAAATCTATATTTTGGGGAAAAGAATAGTA |
| | | CGGGGGTAAATAATCTTTTTAACTTTGTCAATGGAAAAATTGAAGTAGATGGGATT |
| | | ATCAACGGAATTCGAGAAAATAAAATTGGAGGAAATTTATATTTCTTAAGCTCGGA |
| | | AGGGATGGCAGTAGGAAAAAATGGAGTTATCAATGCTGGTTCTTTTCATTCTATTA |
| | | TTCCAAAACAAGATGATTTTAAGAAGGCTTTGGAAGAAGCCAAACATGGTTAA |
| 5 | Lcry9Aa2_{Bacillus} | AGATAACCCAAATAATGTTTTAAAATTTTAAAAATAATGTAGGAGGAAAAATT |
| 6 | YFP | ATGTCCAAGGGCGAGGAGCTGTTCACCGGCGTGGTGCCTATCCTCGTGGAGCTCGA |
| | | CGGCGACGTGAACGGCCACAAGTTCAGCGTGTCCGGCGAGGGCGAGGGCGACGCCA |
| | | CCTACGGCAAGCTGACCCTGAAGTTCATCTGCACCACCGGCAAGCTCCCGGTGCCA |
| | | TGGCCAACCCTGGTGACCACCTTCGGCTACGGCCTGCAGTGCTTCGCCAGGTACCC |
| | | CGACCACATGAAGAGGCACGACTTCTTCAAGAGCGCCATGCCAGAGGGCTACGTGC |
| | | AGGAGAGGACCATCTTCTTCAAGGACGACGGCAACTACAAGACCAGGGCCGAGGTG |
| | | AAGTTCGAGGGCGACACCCTGGTGAACAGGATCGAGCTGAAGGGCATCGACTTCAA |
| | | GGAGGACGGCAACATCCTGGGCCACAAGCTGGAGTACAACTACAACTCCCACAACG |
| | | TGTACATCATGGCCGACAAGCAGAAGAACGGCATCAAGGTGAACTTCAAGATCCGC |
| | | CACAACATCGAGGACGGCTCCGTGCAGCTGGCCGACCACTACCAGCAGAACACCCC |
| | | AATCGGCGACGGCCCGGTGCTCCTCCCTGACAACCACTACCTCAGCTACCAGTCCG |
| | | CCCTCAGCAAGGACCCGAACGAGAAGAGGGACCACATGGTGCTGCTGGAGTTCGTG |
| | | ACCGCCGCCGGCATCACCCACGGCATGGACGAGCTCTACAAGTGA |
| 7 | Trps16_{Nicotiana} | AGAAATTCAATTAAGGAAATAAATTAAGGAAATACAAAAAGGGGGGTAGTCATTTG |
| | | TATATAACTTTGTATGACTTTTCTCTTCTATTTTTTTGTATTTCCTCCCTTTCCTT |
| | | TTCTATTTGTATTTTTTTATCATTGCTTCCATTGAATT |
| 8 | Right | TTCATCCATAGATCCTTTACTCATATTTATTCAATCGGAATACTTATCGGAATACT |
| | Flank_{Sorghum} | TAATCCAATGCAAAATTTTGCTTCGCGACTAGGTAGTCATAATCGAATTTGTATTT |
| | | TAGATGCAAATTCAATTAGTCTTTGGATACTAATCGCGAGAATGTATATTCTTCCT |
| | | CAATATGCTATTGAGAGGAAAAGGATTAAACCCTTTATAAGAACTAAAGTTTTCAT |
| | | CGGAATATGAATATAAAAAAACTTAAGGATGCCTTAAGTATATCATTTCAAATTCA |
| | | GTTATTAATAGAACGAATCACACTTTTACCACTAAACTATACCCGCTACATGTAGA |
| | | TTATGATACCAATGCTACCCTTTGTCAAGGGTAGCCATTCGAGAAGGAGGCTAATT |
| | | CCACCTTATCGAATCAAAGGAGAAAGTTCATGGCGGTGGGCGATTGGTACTTCAAT |
| | | CGCGGGTCTTTACTTTAGGATTTAGATAGCCCCTCTCTAGTCTGTAAAATACATCT |
| | | CTTCTTACCATACCAATAGCGTATGAACCAAATGTATGCATTTCGATTAGGATCTA |
| | | TTCTACGGTTATGACTACAAGGATCATTATTTGTAAGGACGTAAATGTGCCAGACT |
| | | GTTGTCTGGAATCGTTTAATTATTCCTACAATATATACTAAGAGATATAAAGGCAG |
| | | TACAATCCCCTCCCTTTCTTCCTTTTCTTTTTTGTTCAGAATTGAACAAAGAAATT |
| | | GGGAAAGATGTTTTCTTCCTCCACGTATCATGAAGTGCGAGCCATAGGGAAGGAGT |
| | | GAGATGACTTTCACAAATTTATCATAGACTTCGTCTATCGCTTGAGAGAAGCAACA |
| | | AAGAGTAATAACTTAAAAAGAAAAACAGATACGAATCGACAGATTTACCTGATGAA |
| | | AATTGACATCAGAGGACTCTGATGAGGATTCCTCAAACTCTTCATAAAGAGGATCC |
| | | AGAAAGTCTCTGGTTAGTCGAGACCCTCCATTTCCTAATTTCCTCTCTTCTTTTCC |
| | | GCTCAATTCTAGTTTATTAGATTCTTGTTTAAAAGAATCAAAGAAGATGAATAGAA |
| | | CTAAGA |
| 9 | LrbcL_{Nicotiana} | TCGAGTAGACCTTGTTGTTGTGAAAATTCTTAATTCATGAGTTGTAGGGAGGGATT |
| | | T |
| 10 | PL4_{Fusobacteria} | ATGGTAGCAGTTAATAAAATTACACAAAATACTTCTGCACATATAAAAAATAGTAG |
| | | TCAAAATGTACGAAATGCTTTGGTAAAAAGCAAATCTCATTCATCTATTAAAACAA |
| | | TTGGAATTGGAGCTGGAGTTGGAGCTGGAGGAGCTGGAGTGACAGGTTCTGTAGCA |
| | | GTGAATAAGATTGTAAATAATACGATAGCAGAATTAAATCATGCAAAAATCACTGC |
| | | GAAGGGAAATGTCGGAGTTATTACAGAGTCTGATGCGGTAATTGCTAATTATGCAG |
| | | GAACAGTGTCTGGAGGGGCCCGTGCAGCAATAGGAGCCTCAACCAGTGTGAATGAA |
| | | ATTACAGGATCTACAAAAGCATATGTAAAAGATTCTACAGTGATTGCTAAAGAAGA |
| | | AACAGATGATTATATTACTACTCAAGGGCAAGTAGATAAAGTGGTAGATAAAGTAT |
| | | TCAAAAATCTTAATATTAACGAAGACTTATCACAAAAAAGAAAAATAAGTAATAAA |
| | | AAAGGATTTGTTACCAATAGTTCAGCTACTCATACTTTAAAATCTTTATTGGCAAA |
| | | TGCCGCTGGTTCAGGACAAGCCGGAGTGGCAGGAACTGTTAATATCAACAAGGTTT |
| | | ATGGAGAAACAGAAGCTCTTGTAGAAAATTCTATATTAAATGCAAAACATTATTCT |
| | | GTAAAGTCAGGAGATTACACGAATTCAATCGGAGTAGTAGGTTCTGTTGGTGTTGG |
| | | TGGAAATGTAGGAGTAGGAGCTTCTTCTGATACCAATATTATAAAAAGAAATACCA |
| | | AGACAAGAGTTGGAAAAACTACAATGTCTGATGAAGGTTTCGGAGAAGAAGCTGAA |
| | | ATTACAGCAGATTCTAAGCAAGGAATTTCCTCTTTTGGAGTCGGAGTCGCAGCAGC |
| | | CGGGGTAGGAGCCGGAGTGGCAGGAACCGTTTCCGCAAATCAATTTGCAGGAAAGA |
| | | CGGAAGTAGATGTGGAAGAATGA |
| 11 | LatpB_{Nicotiana} | GAATTAACCGATCGACGTGCAAGCGGACATTTATTTTAAATTCGATAATTTTTGCA |
| | | AAAACATTTCGACATATTTATTTATTTTATTATT |
| 12 | DsRed_{Discosoma} | ATGGCCTCCTCCGAGAACGTCATCAAGGAGTTCATGCGCTTCAAGGTGCGCATGGA |
| | | GGGCACCGTGAACGGCCACGAGTTCGAGATCGAGGGCGAGGGCGAGGGCCGCCCCT |
| | | ACGAGGGCCACAACACCGTGAAGCTGAAGGTGACCAAGGGCGGCCCCCTGCCCTTC |
| | | GCCTGGGACATCCTGTCCCCCCAGTTCCAGTACGGCTCCAAGGTGTACGTGAAGCA |
| | | CCCCGCCGACATCCCCGACTACAAGAAGCTGTCCTTCCCCGAGGGCTTCAAGTGGG |
| | | AGCGCGTGATGAACTTCGAGGACGGCGGCGTGGTGACCGTGACCCAGGACTCCTCC |
| | | CTGCAGGACGGCTGCTTCATCTACAAGGTGAAGTTCATCGGCGTGAACTTCCCCTC |
| | | CGACGGCCCCGTAATGCAGAAGAAGACCATGGGCTGGGAGGCCTCCACCGAGCGCC |
| | | TGTACCCCCGCGACGGCGTGCTGAAGGGCGAGATCCACAAGGCCCTGAAGCTGAAG |
| | | GACGGCGGCCACTACCTGGTGGAGTTCAAGTCCATCTACATGGCCAAGAAGCCCGT |
| | | GCAGCTGCCCGGCTACTACTACGTGGACTCCAAGCTGGACATCACCTCCCACAACG |
| | | AGGACTACACCATCGTGGAGCAGTACGAGCGCACCGAGGGCCGCCACCACCTGTTC |
| | | CTGGTACCCTAAGAGCTCCGTAA |
| 13 | Lomega | TATTTTTACAACAATTAGCAACAACAACAAACAACAAACAACATTACAATTAGATT |
| | prime | TACAATTACA |
| | Tobacco | |
| | mosaic virus | |
| 14 | CFP | ATGGTGAGCAAGGGCGAGGAGCTGTTCACCGGGGTGGTGCCCATCCTGGTCGAGCT |
| | | GGACGGCGACGTAAACGGCCACAAGTTCAGCGTGTCCGGCGAGGGCGAGGGCGATG |
| | | CCACCTACGGCAAGCTGACCCTGAAGTTCATCTGCACCACCGGCAAGCTGCCCGTG |
| | | CCCTGGCCCACCCTCGTGACCACCCTGTCCTGGGGCGTGCAGTGCTTCGCCCGCTA |
| | | CCCCGACCACATGAAGCAGCACGACTTCTTCAAGTCCGCCATGCCCGAAGGCTACG |
| | | TCCAGGAGCGCACCATCTTCTTCAAGGACGACGGCAACTACAAGACCCGCGCCGAG |
| | | GTGAAGTTCGAGGGCGACACCCTGGTGAACCGCATCGAGCTGAAGGGCATCGACTT |
| | | CAAGGAGGACGGCAACATCCTGGGGCACAAGCTGGAGTACAACTACATCAGCGACA |
| | | ACGTCTATATCACCGCCGACAAGCAGAAGAACGGCATCAAGGCCAACTTCAAGATC |
| | | CGCCACAACATCGAGGACGGCGGCGTGCAGCTCGCCGACCACTACCAGCAGAACAC |
| | | CCCCATCGGCGACGGCCCCGTGCTGCTGCCCGACAACCACTACCTGAGCACCCAGT |
| | | CCAAGCTGAGCAAAGACCCCAACGAGAAGCGCGATCACATGGTCCTGCTGGAGTTC |
| | | GTGACCGCCGCCGGGATCACTCTCGGCATGGACGAGCTGTACAAGTAA |
| 15 | Left | AGTTCAAGTCTGGCGAGAGTAATATTCTACAACTAACAACTCATTTACTTTGAGAC |
| | Flank_{Panicum} | CGACCCACTTCCTATCTAGAATTTTTTTTACTAGTCCTTTATATTGCAATGTGTCA |
| | | ACCGTCAAATGCTTTGGCAATTTGCCCGGATCGGATGAAGCAATAGAATTTTGAAC |
| | | CAGACGTTTTGATCGTTGGTTATCCTTCGTAGTAATAATATCTCGGGGTTTGCAAC |
| | | GAAAACTTGGTATATCAACTATACGACCATTAACTAAAATATGTCTATGGTTAACT |
| | | AATTGCCGGGCCCCAGGAATGGTTGAAGCCATACCCAATCGAAAAAGTATATTATC |
| | | CAAACGCATTTCAAGTAATTGTAGTAAAACCTGACCTGTGGATCTTTTTGCTTTTC |
| | | CAGCGATATGTAGATATCTAAGTAATTGGCGTTCTGTCAGACCATAATGAAAACGC |
| | | AATTTCTGTTTTTCTTGAAGACGAATACGATATTGCTCTTTTTTCCCAGAATGGAA |
| | | TTTCTTTTTCTGATTACTTCCGGATTTAGGCGTTTTTCTAGTGAGTCCTGGTAAAG |
| | | CTCCCAGACGGCGTATTTTTTTTAAACGAGGCCCTCGATAACGGGACATGAAGACT |
| | | CCTTTTTTTTTATTGAAATTTCATTTTACACAATAAATTTCATTCTATTTACATTA |
| | | CAAAATACATCGAAATTCAAACTGAATTAAACTAAAGGATAAGCAGAGTAAAATCT |
| | | ACTAAAAGTACCACAAAAAATGAAGTAGCACAAAAAATGGAATTTCATCAACATCC |
| | | GGATTTTTTGTATATATATTATTTATTTTTATGCTTTGTATCTAGCAAAATTGTAA |
| | | GGTAGAACGACATAAAAGACCCTGGCTTCCCCATTTAATTTAGAGAAAAAGAGAAA |
| | | TTCTTGTTCATGGAACATCGATAGAGAAAAAAGCCGACTATCGGATTTGAACCGAT |
| | | GACCCTCGCATTACAAATGCGATGCTCTAACCTCTGAGCTAAGTGGGCTTACATAA |
| | | CAGAAATAGTGTAACAAATAGAAATATATATAGGGAATCTGTAAAATGTCAGATCT |
| | | TAATTATTAATCTTAGTTATTAACTAGTTCGAAATTGGAAGTTCTACTTAGAATTA |
| | | GTAAAAGAATTAGTAAAAAAAATACTAGAATTTCATAAAGATAAAATTAGCTTGAT |
| | | ATGCTTAACTAAATGATATTCTTAAATAGGATTCTAGAATTTATTGAACTTTCTTT |
| | | TTATTTCTCTAATTCGCAAATGGATTTTTCTATTCTAATAGAATCTATTCCAAATT |
| | | CTATATTGAATTTGATTTCAGATATTTTCAATTTGATATGGCTCGGACGAATAATC |
| | | TAATACATATAA |
| 16 | Right | AAGAAGAATATATATGAATATTATAATAAAGAGAAAATGCCAAGAGATTAGCATTT |
| | Flank_{Panicum} | TCATTCGATCATTATATACATTTTTGATTTGAGATATTTTGTTTTTTTTTTTATTT |
| | | GTTAATAATTTAAGGATAAATAGTTCACTAAAGAGAAGATAGAATCATAACAAATG |
| | | AAATTTCTAATTCAGATTAGAAAACAAAGAATGAATATCAAGCGTTATAGTATGAT |
| | | TTTGAATACTCTAAAAAAGGAAGGAGGAAGGCGGGGGAGAGAAAAACTTTTGGATA |
| | | TATTCATTCCGATTGAATTGCAAATATATCAACGATAGAATCAATTCAATTCTGAA |
| | | TTGCAATAAGCGAGCGGGCTCTCTCAAATAGAGATGAGCTGCTAGACTACGTCGAA |
| | | TAATCAATTCAATGATTCAAAAAAAACTAAGAGATGGATGAAATTATACAAGGAAT |
| | | CCTGGTTTCAAAGAAAAGGAAAATGGGGATATGGCGAAATCGGTAGACGCTACGGA |
| | | CTTGATTGTATTGAGCCTTGGTATGGAAACCTGCTAAGTGGTAACTTCCAAATTCA |
| | | GAGAAACCCTGGAATGAAAAATGGGCAATCCTGAGCCAAATCCCTTTTTTGAAAAA |
| | | ACAAGTGGTTCTCAAACTAGAACCCAAAGGAAAAGGATAGGTGCAGAGACTCAATG |
| | | GAAGCTGTTCTAACGAATCGAAGTAATTACGTTGTGTTGGTAGTGGAACTCCCTCG |
| | | AAATTATAGAAAGAAGGGCTTTATACATCTAATACACACGTATAGATACTGACATA |
| | | GCAAACGATTAATCATAGAACCCATATCATAATATAGGTTCTTTATTTTATTTTTT |
| | | AAAATGAAATTAGGAATGATTATGAAATATAAAATTCTGAATTTTTTTTAGAATTA |
| | | TTGTGAATCCATTCCAATCGAATATTGAGTAATCAAATCCTTCAATTCATTGTTTT |
| | | GAGATCTTCAAAAAAGTGGATTAATCGAACGAGGATAAAGAGAGAGTCCCATTCTA |
| | | CATGTCAATACTGACAACAATGAAATTTCTAGTAAAAGGAAAATCCGTCGACTTTA |
| | | TAAGTCGTGAGGGTTCAAGTCCCTCTATCCCCAAACCCTCTTTTATTCCCTAACCA |
| | | TAGTAGTTATCCTTTTTTTTCTTTTATCAATGGGTTTAAGATTCATTAGCTTTCTC |
| | | ATTCTACTCTTTCACAAAGGAGTGCTACGAGAACTCAATGAATCTTATGCTATTCA |
| | | TTAAATAGATGATTTCTTTTTTATTTGATAGGATTACCCCGCCCATTTCCAAATTT |
| | | AGAATGGAATACTTTATTGATTTTTTAGTCCCTTTAATTGACATAGATGCAAATAC |
| | | TCTACTAGGATGATGCACAAGAAAG |
| 17 | Construct 1 | AGTATTTAGTAACCCGATACAAAATAAATAAAAAGAAAGGCCCTTTTTTCGAAAAA |
| | | ATTCGGATTTTTATACACATTAAAATGCTATTTTCGTTCCGAATTATTACCTATTC |
| | | TCTTTCATTATTATGAAATTCTATTGCCATTAGAATATTGATTGATAGACAATTAA |
| | | TAAAAAGAAAACTCTAATAGAAAATGAAACGGTCGACCCAGACATAGACGGTCGAC |
| | | CCGGGTGGATATACCCTATAAAATATAGGCCGTAGCAAGCGTAGTTCAATGTAGCG |
| | | AGCGTAGTTCAGTGGTAAAACATCTCCTTGCCAAGGAGAAGATACGGGTTCGATTC |
| | | CCGTCGCTCGCCAGCTTAATTTAGTAAGGTGCTATGATAAAAAATTCAGTTTAGTT |
| | | GACTACTTAACAACTTCTATTAAATTACTATTTAATATTAAATGAATATGAAATTC |
| | | AGTAGTTGTTAGTCTAGTACTGTACCCCTTCCTATCTTATCCTTCCTTTGCACCCG |
| | | ACTCAAAAAAAAGAGTGCTTCGAGGGCGCAAATTCAACTTTCTAAGAAAGTTCCCT |
| | | AAACCGGGGATTCGCCGAGACAAACAAGAGACAAACGGTTTTGAAAGGGGGATAGG |
| | | CTATGCTTTTCTTTCATTTTTTTTTCTGCCTGCTGAATAAAAAAAGGGTTGGATAT |
| | | AGCCCTCTATCATATATATAGAAATAGAATAGTCCATTTATACGGAGTGCTAAGTG |
| | | CGGAGACGGGAATCGAACCCGTGACCTCAAGGTTATGAGCCTCGTGAGCTACCAAA |
| | | CTGCTCTACTCCGCTCTGTAGGGCCGAAAACTGGTGGACGAAAGAAAAAGGTTGAA |
| | | TAGAAGTCTCTACCATGTCTAGACAAACAAATGGAATAGTCTTTTTATACAGAATG |
| | | GAGCGGGTAGCGGGAATCGAACCCGCATCGTTAGCTTGGAAGGCTAGGGGTTATAG |
| | | TCGACGTTGGTTGATTATTTTTGACGTCTCTAATTCAAAACCGAACATGAAATTTT |
| | | GATTTCATTCGGCTCCTTTATGGATATTCTCACCACTTAACATCTATGTCAGCTTT |
| | | TCTGTCTGAATGGAACCAAAGCTCTCTGCTTTCTAGATGATCCTTATAGAGTAGGA |
| | | GATAGAAATTCTCCTAAATATCTATCTAATCTACTTACTTCGTTCCCTAATTTCAT |
| | | TCAAGAGATCCTGAGGAAAAGAGTTGGGTTTCCACCGAGCTGAAACAATATAATAT |
| | | ACTGATGGTTCTAGTAAACCAAAACCATCGTTTTTTAGCTATTGGGCTTCCATTTC |
| | | CTACAAAACAAAAGAAGATTTAGTTACGATTGGAAATAAACTTTTTTGTATCGGTA |
| | | CCCCAAAGCTCCCCCGCCGTCGTTCAATGAGAATGGATAAGAGGCTCGTGGGATTG |
| | | ACGTGAGGGGGCAGGGATGGCTATATTTCTGGGAGCGAACTCCGGGCGAATACGAA |
| | | GCGCTTGGATACGGGGAGACCACAACGGTTTCCCTCTAGAAATAATTTTGTTTAAC |
| | | TTTAAGAAGGAGATATACCCATGAGCGGCATCAAAAGTAACGTTCAGAGGACAAGG |
| | | AAGAGGATATCAGATTCTAAAAAAGTTTTAATGATTTTGGGATTGTTGATTAACAC |
| | | TATGACGGTGAGGGCTAATGATACAATCGCCGCGACTGAGAATTTTGGAACAAAAA |
| | | TAGAAAAAAAGGATAATGTTTATGACATTACTACAAACAAGATTCAAGGGGAGAAC |
| | | GCTTTTAACAGTTTTAATAGATTTGCTTTAACAGAAAATAATATAGCAAATCTATA |
| | | TTTTGGGGAAAAGAATAGTACGGGGGTAAATAATCTTTTTAACTTTGTCAATGGAA |
| | | AAATTGAAGTAGATGGGATTATCAACGGAATTCGAGAAAATAAAATTGGAGGAAAT |
| | | TTATATTTCTTAAGCTCGGAAGGGATGGCAGTAGGAAAAAATGGAGTTATCAATGC |
| | | TGGTTCTTTTCATTCTATTATTCCAAAACAAGATGATTTTAAGAAGGCTTTGGAAG |
| | | AAGCCAAACATGGTTAAAGATAACCCAAATAATGTTTTAAAATTTTAAAAATAATG |
| | | TAGGAGGAAAAATTATGTCCAAGGGCGAGGAGCTGTTCACCGGCGTGGTGCCTATC |
| | | CTCGTGGAGCTCGACGGCGACGTGAACGGCCACAAGTTCAGCGTGTCCGGCGAGGG |
| | | CGAGGGCGACGCCACCTACGGCAAGCTGACCCTGAAGTTCATCTGCACCACCGGCA |
| | | AGCTCCCGGTGCCATGGCCAACCCTGGTGACCACCTTCGGCTACGGCCTGCAGTGC |
| | | TTCGCCAGGTACCCCGACCACATGAAGAGGCACGACTTCTTCAAGAGCGCCATGCC |
| | | AGAGGGCTACGTGCAGGAGAGGACCATCTTCTTCAAGGACGACGGCAACTACAAGA |
| | | CCAGGGCCGAGGTGAAGTTCGAGGGCGACACCCTGGTGAACAGGATCGAGCTGAAG |
| | | GGCATCGACTTCAAGGAGGACGGCAACATCCTGGGCCACAAGCTGGAGTACAACTA |
| | | CAACTCCCACAACGTGTACATCATGGCCGACAAGCAGAAGAACGGCATCAAGGTGA |
| | | ACTTCAAGATCCGCCACAACATCGAGGACGGCTCCGTGCAGCTGGCCGACCACTAC |
| | | CAGCAGAACACCCCAATCGGCGACGGCCCGGTGCTCCTCCCTGACAACCACTACCT |
| | | CAGCTACCAGTCCGCCCTCAGCAAGGACCCGAACGAGAAGAGGGACCACATGGTGC |
| | | TGCTGGAGTTCGTGACCGCCGCCGGCATCACCCACGGCATGGACGAGCTCTACAAG |
| | | TGAAGAAATTCAATTAAGGAAATAAATTAAGGAAATACAAAAAGGGGGGTAGTCAT |
| | | TTGTATATAACTTTGTATGACTTTTCTCTTCTATTTTTTTGTATTTCCTCCCTTTC |
| | | CTTTTCTATTTGTATTTTTTTATCATTGCTTCCATTGAATTTTCATCCATAGATCC |
| | | TTTACTCATATTTATTCAATCGGAATACTTATCGGAATACTTAATCCAATGCAAAA |
| | | TTTTGCTTCGCGACTACGTACTCATAATCGAATTTGTATTTTAGATGCAAATTCAA |
| | | TTAGTCTTTGGATACTAATCGCGAGAATGTATATTCTTCCTCAATATGCTATTGAG |
| | | AGGAAAAGGATTAAACCCTTTATAAGAACTAAAGTTTTCATCGGAATATGAATATA |
| | | AAAAAACTTAAGGATGCCTTAAGTATATCATTTCAAATTCAGTTATTAATAGAACG |
| | | AATCACACTTTTACCACTAAACTATACCCGCTACATGTAGATTATGATACCAATGC |
| | | TACCCTTTGTCAAGGGTAGCCATTCGAGAAGGAGGCTAATTCCACCTTATCGAATC |
| | | AAAGGAGAAAGTTCATGGCGGTGGGCGATTGGTACTTCAATCGCGGGTCTTTACTT |
| | | TAGGATTTAGATAGCCCCTCTCTAGTCTGTAAAATACATCTCTTCTTACCATACCA |
| | | ATAGCGTATGAACCAAATGTATGCATTTCGATTAGGATCTATTCTACGGTTATGAC |
| | | TACAAGGATCATTATTTGTAAGGACGTAAATGTGCCAGACTGTTGTCTGGAATCGT |
| | | TTAATTATTCCTACAATATATACTAAGAGATATAAAGGCAGTACAATCCCCTCCCT |
| | | TTCTTCCTTTTCTTTTTTGTTCAGAATTGAACAAAGAAATTGGGAAAGATGTTTTC |
| | | TTCCTCCACGTATCATGAAGTGCGAGCCATAGGGAAGGAGTGAGATGACTTTCACA |
| | | AATTTATCATAGACTTCGTCTATCGCTTGAGAGAAGCAACAAAGAGTAATAACTTA |
| | | AAAAGAAAAACAGATACGAATCGACAGATTTACCTGATGAAAATTGACATCAGAGG |
| | | ACTCTGATGAGGATTCCTCAAACTCTTCATAAAGAGGATCCAGAAAGTCTCTGGTT |
| | | AGTCGAGACCCTCCATTTCCTAATTTCCTCTCTTCTTTTCCGCTCAATTCTAGTTT |
| | | ATTAGATTCTTGTTTAAAAGAATCAAAGAAGATGAATAGAACTAAGA |
| 18 | Construct 2 | AGTATTTAGTAACCCGATACAAAATAAATAAAAAGAAAGGCCCTTTTTTCGAAAAA |
| | | ATTCGGATTTTTATACACATTAAAATGCTATTTTCGTTCCGAATTATTACCTATTC |
| | | TCTTTCATTATTATGAAATTCTATTGCCATTAGAATATTGATTGATAGACAATTAA |
| | | TAAAAAGAAAACTCTAATAGAAAATGAAACGGTCGACCCAGACATAGACGGTCGAC |
| | | CCGGGTGGATATACCCTATAAAATATAGGCCGTAGCAAGCGTAGTTCAATGTAGCG |
| | | AGCGTAGTTCAGTGGTAAAACATCTCCTTGCCAAGGAGAAGATACGGGTTCGATTC |
| | | CCGTCGCTCGCCAGCTTAATTTAGTAAGGTGCTATGATAAAAAATTCAGTTTAGTT |
| | | GACTACTTAACAACTTCTATTAAATTACTATTTAATATTAAATGAATATGAAATTC |
| | | AGTAGTTGTTAGTCTAGTACTGTACCCCTTCCTATCTTATCCTTCCTTTGCACCCG |
| | | ACTCAAAAAAAAGAGTGCTTCGAGGGCGCAAATTCAACTTTCTAAGAAAGTTCCCT |
| | | AAACCGGGGATTCGCCGAGACAAACAAGAGACAAACGGTTTTGAAAGGGGGATAGG |
| | | CTATGCTTTTCTTTCATTTTTTTTTCTGCCTGCTGAATAAAAAAAGGGTTGGATAT |
| | | AGCCCTCTATCATATATATAGAAATAGAATAGTCCATTTATACGGACTGCTAAGTG |
| | | CGGAGACGGGAATCGAACCCGTGACCTCAAGGTTATGAGCCTCGTGAGCTACCAAA |
| | | CTGCTCTACTCCGCTCTGTAGGGCCGAAAACTGGTGGACGAAAGAAAAAGGTTGAA |
| | | TAGAAGTCTCTACCATGTCTAGACAAACAAATGGAATAGTCTTTTTATACAGAATG |
| | | GAGCGGGTAGCGGGAATCGAACCCGCATCGTTAGCTTGGAAGGCTAGGGGTTATAG |
| | | TCGACGTTGGTTGATTATTTTTGACGTCTCTAATTCAAAACCGAACATGAAATTTT |
| | | GATTTCATTCGGCTCCTTTATGGATATTCTCACCACTTAACATCTATGTCAGCTTT |
| | | TCTGTCTGAATGGAACCAAAGCTCTCTGCTTTCTAGATGATCCTTATAGAGTAGGA |
| | | GATAGAAATTCTCCTAAATATCTATCTAATCTACTTACTTCGTTCCCTAATTTCAT |
| | | TCAAGAGATCCTGAGGAAAAGAGTTGGGTTTCCACCGAGCTGAAACAATATAATAT |
| | | ACTGATGGTTCTAGTAAACCAAAACCATCGTTTTTTAGCTATTGGGCTTCCATTTC |
| | | CTACAAAACAAAAGAAGATTTAGTTACGATTGGAAATAAACTTTTTTGTATCGGGC |
| | | AACCCACTAGCATATCGAAATTCTAATTTTCTGTAGAGAAGTCCGTATTTTTCCAA |
| | | TCAACTTCATTAAAAATTTGAATAGATCTACATACACCTTGGTTGACACGAGTATA |
| | | TAAGTCATGTTATACTGTTGAATAACAAGCCTTCCATTTTCTATTTTGATTTGTAG |
| | | AAAACTAGTGTGCTTGGGAGTCCCTGATGATTAAATAAACCAAGATTTTACCATGG |
| | | CATCGAGTAGACCTTGTTGTTGTGAAAATTCTTAATTCATGAGTTGTAGGGAGGGA |
| | | TTTATGGTAGCAGTTAATAAAATTACACAAAATACTTCTGCACATATAAAAAATAG |
| | | TACTCAAAATGTACGAAATGCTTTGGTAAAAAGCAAATCTCATTCATCTATTAAAA |
| | | CAATTGGAATTGGAGCTGGAGTTGGAGCTGGAGGAGCTGGAGTGACAGGTTCTGTA |
| | | GCAGTGAATAAGATTGTAAATAATACGATAGCAGAATTAAATCATGCAAAAATCAC |
| | | TGCGAAGGGAAATGTCGGAGTTATTACAGAGTCTGATGCGGTAATTGCTAATTATG |
| | | CAGGAACAGTGTCTGGAGGGGCCCGTGCAGCAATAGGAGCCTCAACCAGTGTGAAT |
| | | GAAATTACAGGATCTACAAAAGCATATGTAAAAGATTCTACAGTGATTGCTAAAGA |
| | | AGAAACAGATGATTATATTACTACTCAAGGGCAAGTAGATAAAGTGGTAGATAAAG |
| | | TATTCAAAAATCTTAATATTAACGAAGACTTATCACAAAAAAGAAAAATAAGTAAT |
| | | AAAAAAGGATTTGTTACCAATAGTTCAGCTACTCATACTTTAAAATCTTTATTGGC |
| | | AAATGCCGCTGGTTCAGGACAAGCCGGAGTGGCAGGAACTGTTAATATCAACAAGG |
| | | TTTATGGAGAAACAGAAGCTCTTGTAGAAAATTCTATATTAAATGCAAAACATTAT |
| | | TCTGTAAAGTCAGGAGATTACACGAATTCAATCGGAGTAGTAGGTTCTGTTGGTGT |
| | | TGGTGGAAATGTAGGAGTAGGAGCTTCTTCTGATACCAATATTATAAAAAGAAATA |
| | | CCAAGACAAGAGTTGGAAAAACTACAATGTCTGATGAAGGTTTCGGAGAAGAAGCT |
| | | GAAATTACAGCAGATTCTAAGCAAGGAATTTCCTCTTTTGGAGTCGGAGTCGCAGC |
| | | AGCCGGGGTAGGAGCCGGAGTGGCAGGAACCGTTTCCGCAAATCAATTTGCAGGAA |
| | | AGACGGAAGTAGATGTGGAAGAATGAGAATTAACCGATCGACGTGCAAGCGGACAT |
| | | TTATTTTAAATTCGATAATTTTTGCAAAAACATTTCGACATATTTATTTATTTTAT |
| | | TATTATGGCCTCCTCCGAGAACGTCATCAAGGAGTTCATGCGCTTCAAGGTGCGCA |
| | | TGGAGGGCACCGTGAACGGCCACGAGTTCGAGATCGAGGGCGAGGGCGAGGGCCGC |
| | | CCCTACGAGGGCCACAACACCGTGAAGCTGAAGGTGACCAAGGGCGGCCCCCTGCC |
| | | CTTCGCCTGGGACATCCTGTCCCCCCAGTTCCAGTACGGCTCCAAGGTGTACGTGA |
| | | AGCACCCCGCCGACATCCCCGACTACAAGAAGCTGTCCTTCCCCGAGGGCTTCAAG |
| | | TGGGAGCGCGTGATGAACTTCGAGGACGGCGGCGTGGTGACCGTGACCCAGGACTC |
| | | CTCCCTGCAGGACGGCTGCTTCATCTACAAGGTGAAGTTCATCGGCGTGAACTTCC |
| | | CCTCCGACGGCCCCGTAATGCAGAAGAAGACCATGGGCTGGGAGGCCTCCACCGAG |
| | | CGCCTGTACCCCCGCGACGGCGTGCTGAAGGGCGAGATCCACAAGGCCCTGAAGCT |
| | | GAAGGACGGCGGCCACTACCTGGTGGAGTTCAAGTCCATCTACATGGCCAAGAAGC |
| | | CCGTGCAGCTGCCCGGCTACTACTACGTGGACTCCAAGCTGGACATCACCTCCCAC |
| | | AACGAGGACTACACCATCGTGGAGCAGTACGAGCGCACCGAGGGCCGCCACCACCT |
| | | GTTCCTGGTACCCTAAGAGCTCCGTAAAAGAAATTCAATTAAGGAAATAAATTAAG |
| | | GAAATACAAAAAGGGGGGTAGTCATTTGTATATAACTTTGTATGACTTTTCTCTTC |
| | | TATTTTTTTGTATTTCCTCCCTTTCCTTTTCTATTTGTATTTTTTTATCATTGCTT |
| | | CCATTGAATTTTCATCCATAGATCCTTTACTCATATTTATTCAATCGGAATACTTA |
| | | TCGGAATACTTAATCCAATGCAAAATTTTGCTTCGCGACTACGTACTCATAATCGA |
| | | ATTTGTATTTTAGATGCAAATTCAATTAGTCTTTGGATACTAATCGCGAGAATGTA |
| | | TATTCTTCCTCAATATGCTATTGAGAGGAAAAGGATTAAACCCTTTATAAGAACTA |
| | | AAGTTTTCATCGGAATATGAATATAAAAAAACTTAAGGATGCCTTAAGTATATCAT |
| | | TTCAAATTCAGTTATTAATAGAACGAATCACATTTTACCACTAAACTATACCCGCT |
| | | ACATGTAGATTATGATACCAATGCTACCCTTTGTCAAGGGTAGCCATTCGAGAAGG |
| | | AGGCTAATTCCACCTTATCGAATCAAAGGAGAAAGTTCATGGCGGTGGGCGATTGG |
| | | TACTTCAATCGCGGGTCTTTACTTTAGGATTTAGATAGCCCCTCTCTAGTCTGTAA |
| | | AATACATCTCTTCTTACCATACCAATAGCGTATGAACCAAATGTATGCATTTCGAT |
| | | TAGGATCTATTCTACGGTTATGACTAGAAGGATCATTATTTGTAAGGACGTAAATG |
| | | TGCCAGACTGTTGTCTGGAATCGTTTAATTATTCCTAGAATATATACTAAGAGATA |
| | | TAAAGGCAGTACAATCCCCTCCCTTTCTTCCTTTTCTTTTTTGTTCAGAATTGAAC |
| | | AAAGAAATTGGGAAAGATGTTTTCTTCCTCCACGTATCATGAAGTGCGAGCCATAG |
| | | GGAAGGAGTGAGATGACTTTCACAAATTTATCATAGACTTCGTCTATCGCTTGAGA |
| | | GAAGCAACAAAGAGTAATAACTTAAAAAGAAAAACAGATACGAATCGACAGATTTA |
| | | CCTGATGAAAATTGACATCAGAGGACTCTGATGAGGATTCCTCAAACTCTTCATAA |
| | | AGAGGATCCAGAAAGTCTCTGGTTAGTCGAGACCCTCCATTTCCTAATTTCCTCTC |
| | | TTCTTTTCCGCTCAATTCTAGTTTATTAGATTCTTGTTTAAAAGAATCAAAGAAGA |
| | | TGAATAGAACTAAGA |
| 19 | Construct 3 | AGTATTTAGTAACCCGATACAAAATAAATAAAAAGAAAGGCCCTTTTTTCGAAAAA |
| | | ATTCGGATTTTTATACACATTAAAATGCTATTTTCGTTCCGAATTATTACCTATTC |
| | | TCTTTCATTATTATGAAATTCTATTGCCATTAGAATATTGATTGATAGACAATTAA |
| | | TAAAAAGAAAACTCTAATAGAAAATGAAACGGTCGACCCAGACATAGACGGTCGAC |
| | | CCGGGTGGATATACCCTATAAAATATAGGCCGTAGCAAGCGTAGTTCAATGTAGCG |
| | | AGCGTAGTTCAGTGGTAAAACATCTCCTTGCCAAGGAGAAGATACGGGTTCGATTC |
| | | CCGTCGCTCGCCAGCTTAATTTAGTAAGGTGCTATGATAAAAAATTCAGTTTAGTT |
| | | GAGTAGTTAACAACTTCTATTAAATTAGTATTTAATATTAAATGAATATGAAATTC |
| | | AGTAGTTGTTAGTCTAGTACTGTACCCCTTCCTATCTTATCCTTCCTTTGCACCCG |
| | | ACTCAAAAAAAAGAGTGCTTCGAGGGCGCAAATTCAACTTTCTAAGAAAGTTCCCT |
| | | AAACCGGGGATTCGCCGAGACAAACAAGAGACAAACGGTTTTGAAAGGGGGATAGG |
| | | CTATGCTTTTCTTTCATTTTTTTTTCTGCCTGCTGAATAAAAAAAGGGTTGGATAT |
| | | AGCCCTCTATCATATATATAGAAATAGAATAGTCCATTTATACGGAGTGCTAAGTG |
| | | CGGAGACGGGAATCGAACCCGTGACCTCAAGGTTATGAGCCTCGTGAGCTACCAAA |
| | | CTGCTCTACTCCGCTCTGTAGGGCCGAAAACTGGTGGACGAAAGAAAAAGGTTGAA |
| | | TACAAGTCTCTACCATGTCTAGACAAACAAATGGAATAGTCTTTTTATACAGAATG |
| | | GAGCGGGTAGCGGGAATCGAACCCGCATCGTTAGCTTGGAAGGCTAGGGGTTATAG |
| | | TCGACGTTGGTTGATTATTTTTGACGTCTCTAATTCAAAACCGAACATGAAATTTT |
| | | GATTTCATTCGGCTCCTTTATGGATATTCTCACCACTTAACATCTATGTCAGCTTT |
| | | TCTGTCTGAATGGAACCAAAGCTCTCTGCTTTCTAGATGATCCTTATAGAGTAGGA |
| | | GATAGAAATTCTCCTAAATATCTATCTAATCTACTTACTTCGTTCCCTAATTTCAT |
| | | TCAAGAGATCCTGAGGAAAAGAGTTGGGTTTCCACCGAGCTGAAACAATATAATAT |
| | | ACTGATGGTTCTAGTAAACCAAAACCATCGTTTTTTAGCTATTGGGCTTCCATTTC |
| | | CTACAAAACAAAAGAAGATTTAGTTACGATTGGAAATAAACTTTTTTGTATCGGTA |
| | | CCCCAAAGCTCCCCCGCCGTCGTTCAATGAGAATGGATAAGAGGCTCGTGGGATTG |
| | | ACGTGAGGGGGCAGGGATGGCTATATTTCTGGGAGCGAACTCCGGGCGAATACGAA |
| | | GCGCTTGGATACGGGGAGACCACAACGGTTTCCCTCTAGAAATAATTTTGTTTAAC |
| | | TTTAAGAAGGAGATATACCCATGAGCGGCATCAAAAGTAACGTTCAGAGGACAAGG |
| | | AAGAGGATATCAGATTCTAAAAAAGTTTTAATGATTTTGGGATTGTTGATTAACAC |
| | | TATGACGGTGAGGGCTAATGATACAATCGCCGCGACTGAGAATTTTGGAACAAAAA |
| | | TAGAAAAAAAGGATAATGTTTATGACATTACTACAAACAAGATTCAAGGGGAGAAC |
| | | GCTTTTAACAGTTTTAATAGATTTGCTTTAACAGAAAATAATATAGCAAATCTATA |
| | | TTTTGGGGAAAAGAATAGTACGGGGGTAAATAATCTTTTTAACTTTGTCAATGGAA |
| | | AAATTGAAGTAGATGGGATTATCAACGGAATTCGAGAAAATAAAATTGGAGGAAAT |
| | | TTATATTTCTTAAGCTCGGAAGGGATGGCAGTAGGAAAAAATGGAGTTATCAATGC |
| | | TGGTTCTTTTCATTCTATTATTCCAAAACAAGATGATTTTAAGAAGGCTTTGGAAG |
| | | AAGCCAAACATGGTTAATCGAGTAGACCTTGTTGTTGTGAAAATTCTTAATTCATG |
| | | AGTTGTAGGGAGGGATTTATGGTAGCAGTTAATAAAATTACACAAAATACTTCTGC |
| | | ACATATAAAAAATAGTACTCAAAATGTACGAAATGCTTTGGTAAAAAGCAAATCTC |
| | | ATTCATCTATTAAAACAATTGGAATTGGAGCTGGAGTTGGAGCTGGAGGAGCTGGA |
| | | GTGACAGGTTCTGTAGCAGTGAATAAGATTGTAAATAATACGATAGCAGAATTAAA |
| | | TCATGCAAAAATCACTGCGAAGGGAAATGTCGGAGTTATTACAGAGTCTGATGCGG |
| | | TAATTGCTAATTATGCAGGAACAGTGTCTGGAGGGGCCCGTGCAGCAATAGGAGCC |
| | | TCAACCAGTGTGAATGAAATTACAGGATCTACAAAAGCATATGTAAAAGATTCTAC |
| | | AGTGATTGCTAAAGAAGAAACAGATGATTATATTACTACTCAAGGGCAAGTAGATA |
| | | AAGTGGTAGATAAAGTATTCAAAAATCTTAATATTAACGAAGACTTATCACAAAAA |
| | | AGAAAAATAAGTAATAAAAAAGGATTTGTTACCAATAGTTCAGCTACTCATACTTT |
| | | AAAATCTTTATTGGCAAATGCCGCTGGTTCAGGACAAGCCGGAGTGGCAGGAACTG |
| | | TTAATATCAACAAGGTTTATGGAGAAACAGAAGCTCTTGTAGAAAATTCTATATTA |
| | | AATGCAAAACATTATTCTGTAAAGTCAGGAGATTACACGAATTCAATCGGAGTAGT |
| | | AGGTTCTGTTGGTGTTGGTGGAAATGTAGGAGTAGGAGCTTCTTCTGATACCAATA |
| | | TTATAAAAAGAAATACCAAGACAAGAGTTGGAAAAACTACAATGTCTGATGAAGGT |
| | | TTCGGAGAAGAAGCTGAAATTACAGCAGATTCTAAGCAAGGAATTTCCTCTTTTGG |
| | | AGTCGGAGTCGCAGCAGCCGGGGTAGGAGCCGGAGTGGCAGGAACCGTTTCCGCAA |
| | | ATCAATTTGCAGGAAAGACGGAAGTAGATGTGGAAGAATGATATTTTTACAACAAT |
| | | TACCAACAACAACAAACAACAAACAACATTACAATTACATTTACAATTACAATGGT |
| | | GAGCAAGGGCGAGGAGCTGTTCACCGGGGTGGTGCCCATCCTGGTCGAGCTGGACG |
| | | GCGACGTAAACGGCCACAAGTTCAGCGTGTCCGGCGAGGGCGAGGGCGATGCCACC |
| | | TACGGCAAGCTGACCCTGAAGTTCATCTGCACCACCGGCAAGCTGCCCGTGCCCTG |
| | | GCCCACCCTCGTGACCACCCTGTCCTGGGGCGTGCAGTGCTTCGCCCGCTACCCCG |
| | | ACCACATGAAGCAGCACGACTTCTTCAAGTCCGCCATGCCCGAAGGCTACGTCCAG |
| | | GAGCGCACCATCTTCTTCAAGGACGACGGCAACTACAAGACCCGCGCCGAGGTGAA |
| | | GTTCGAGGGCGACACCCTGGTGAACCGCATCGAGCTGAAGGGCATCGACTTCAAGG |
| | | AGGACGGCAACATCCTGGGGCACAAGCTGGAGTACAACTACATCAGCGACAACGTC |
| | | TATATCACCGCCGACAAGCAGAAGAACGGCATCAAGGCCAACTTCAAGATCCGCCA |
| | | CAACATCGAGGACGGCGGCGTGCAGCTCGCCGACCACTACCAGCAGAACACCCCCA |
| | | TCGGCGACGGCCCCGTGCTGCTGCCCGACAACCACTACCTGAGCACCCAGTCCAAG |
| | | CTGAGCAAAGACCCCAACGAGAAGCGCGATCACATGGTCCTGCTGGAGTTCGTGAC |
| | | CGCCGCCGGGATCACTCTCGGCATGGACGAGCTGTACAAGTAAAGAAATTCAATTA |
| | | AGGAAATAAATTAAGGAAATACAAAAAGGGGGGTAGTCATTTGTATATAACTTTGT |
| | | ATGACTTTTCTCTTCTATTTTTTTGTATTTCCTCCCTTTCCTTTTCTATTTGTATT |
| | | TTTTTATCATTGCTTCCATTGAATTTTCATCCATAGATCCTTTACTCATATTTATT |
| | | CAATCGGAATACTTATCGGAATACTTAATCCAATGCAAAATTTTGCTTCGCGACTA |
| | | CGTACTCATAATCGAATTTGTATTTTAGATGCAAATTCAATTAGTCTTTGGATACT |
| | | AATCGCGAGAATGTATATTCTTCCTCAATATGCTATTGAGAGGAAAAGGATTAAAC |
| | | CCTTTATAAGAACTAAAGTTTTCATCGGAATATGAATATAAAAAAACTTAAGGATG |
| | | CCTTAAGTATATCATTTCAAATTCAGTTATTAATAGAACGAATCACATTTTACCAC |
| | | TAAACTATACCCGCTACATGTAGATTATGATACCAATGCTACCCTTTGTCAAGGGT |
| | | AGCCATTCGAGAAGGAGGCTAATTCCACCTTATCGAATCAAAGGAGAAAGTTCATG |
| | | GCGGTGGGCGATTGGTACTTCAATCGCGGGTCTTTACTTTAGGATTTAGATAGCCC |
| | | CTCTCTAGTCTGTAAAATACATCTCTTCTTACCATACCAATAGCGTATGAACCAAA |
| | | TGTATGCATTTCGATTAGGATCTATTCTACGGTTATGACTACAAGGATCATTATTT |
| | | GTAAGGACGTAAATGTGCCAGACTGTTGTCTGGAATCGTTTAATTATTCCTACAAT |
| | | ATATACTAAGAGATATAAAGGCAGTACAATCCCCTCCCTTTCTTCCTTTTCTTTTT |
| | | TGTTCAGAATTGAACAAAGAAATTGGGAAAGATGTTTTCTTCCTCCACGTATCATG |
| | | AAGTGCGAGCCATAGGGAAGGAGTGAGATGACTTTCACAAATTTATCATAGACTTC |
| | | GTCTATCGCTTGAGAGAAGCAACAAAGAGTAATAACTTAAAAAGAAAAACAGATAC |
| | | GAATCGACAGATTTACCTGATGAAAATTGACATCAGAGGACTCTGATGAGGATTCC |
| | | TCAAACTCTTCATAAAGAGGATCCAGAAAGTCTCTGGTTAGTCGAGACCCTCCATT |
| | | TCCTAATTTCCTCTCTTCTTTTCCGCTCAATTCTAGTTTATTAGATTCTTGTTTAA |
| | | AAGAATCAAAGAAGATGAATAGAACTAAGA |
| 20 | Construct 4 | AGTTCAAGTCTGGCGAGAGTAATATTCTACAACTAACAACTCATTTACTTTGAGAC |
| | | CGACCCACTTCCTATCTAGAATTTTTTTTACTAGTCCTTTATATTGCAATGTGTCA |
| | | ACCGTCAAATGCTTTGGCAATTTGCCCGGATCGGATGAAGCAATAGAATTTTGAAC |
| | | CAGACGTTTTGATCGTTGGTTATCCTTCGTAGTAATAATATCTCGGGGTTTGCAAC |
| | | GAAAACTTGGTATATCAACTATACGACCATTAACTAAAATATGTCTATGGTTAACT |
| | | AATTGCCGGGCCCCAGGAATGGTTGAAGCCATACCCAATCGAAAAAGTATATTATC |
| | | CAAACGCATTTCAAGTAATTGTAGTAAAACCTGACCTGTGGATCTTTTTGCTTTTC |
| | | CAGCGATATGTAGATATCTAAGTAATTGGCGTTCTGTCAGACCATAATGAAAACGC |
| | | AATTTCTGTTTTTCTTGAAGACGAATACGATATTGCTCTTTTTTCCCAGAATGGAA |
| | | TTTCTTTTTCTGATTACTTCCGGATTTAGGCGTTTTTCTAGTGAGTCCTGGTAAAG |
| | | CTCCCAGACGGCGTATTTTTTTTAAACGAGGCCCTCGATAACGGGACATGAAGACT |
| | | CCTTTTTTTTTATTGAAATTTCATTTTACACAATAAATTTCATTCTATTTACATTA |
| | | CAAAATACATCGAAATTCAAACTGAATTAAACTAAAGGATAAGCAGAGTAAAATCT |
| | | ACTAAAAGTACCACAAAAAATGAAGTAGCACAAAAAATGGAATTTCATCAACATCC |
| | | GGATTTTTTGTATATATATTATTTATTTTTATGCTTTGTATCTAGCAAAATTGTAA |
| | | GGTAGAACGACATAAAAGACCCTGGCTTCCCCATTTAATTTAGAGAAAAAGAGAAA |
| | | TTCTTGTTCATGGAACATCGATAGAGAAAAAAGCCGACTATCGGATTTGAACCGAT |
| | | GACCCTCGCATTACAAATGCGATGCTCTAACCTCTGAGCTAAGTGGGCTTACATAA |
| | | CAGAAATAGTGTAACAAATAGAAATATATATAGGGAATCTGTAAAATGTCAGATCT |
| | | TAATTATTAATCTTAGTTATTAACTAGTTCGAAATTGGAAGTTCTACTTAGAATTA |
| | | GTAAAAGAATTAGTAAAAAAAATACTAGAATTTCATAAAGATAAAATTAGCTTGAT |
| | | ATGCTTAACTAAATGATATTCTTAAATAGGATTCTAGAATTTATTGAACTTTCTTT |
| | | TTATTTCTCTAATTCGCAAATGGATTTTTCTATTCTAATAGAATCTATTCCAAATT |
| | | CTATATTGAATTTGATTTCAGATATTTTCAATTTGATATGGCTCGGACGAATAATC |
| | | TAATACATATAAGGTACCCCAAAGCTCCCCCGCCGTCGTTCAATGAGAATGGATAA |
| | | GAGGCTCGTGGGATTGACGTGAGGGGGCAGGGATGGCTATATTTCTGGGAGCGAAC |
| | | TCCGGGCGAATACGAAGCGCTTGGATACGGGGAGACCACAACGGTTTCCCTCTAGA |
| | | AATAATTTTGTTTAACTTTAAGAAGGAGATATACCCATGAGCGGCATCAAAAGTAA |
| | | CGTTCAGAGGACAAGGAAGAGGATATCAGATTCTAAAAAAGTTTTAATGATTTTGG |
| | | GATTGTTGATTAACACTATGACGGTGAGGGCTAATGATACAATCGCCGCGACTGAG |
| | | AATTTTGGAACAAAAATAGAAAAAAAGGATAATGTTTATGACATTACTACAAACAA |
| | | GATTCAAGGGGAGAACGCTTTTAACAGTTTTAATAGATTTGCTTTAACAGAAAATA |
| | | ATATAGCAAATCTATATTTTGGGGAAAAGAATAGTACGGGGGTAAATAATCTTTTT |
| | | AACTTTGTCAATGGAAAAATTGAAGTAGATGGGATTATCAACGGAATTCGAGAAAA |
| | | TAAAATTGGAGGAAATTTATATTTCTTAAGCTCGGAAGGGATGGCAGTAGGAAAAA |
| | | ATGGAGTTATCAATGCTGGTTCTTTTCATTCTATTATTCCAAAACAAGATGATTTT |
| | | AAGAAGGCTTTGGAAGAAGCCAAACATGGTTAATCGAGTAGACCTTGTTGTTGTGA |
| | | AAATTCTTAATTCATGAGTTGTAGGGAGGGATTTATGGTAGCAGTTAATAAAATTA |
| | | CACAAAATACTTCTGCACATATAAAAAATAGTACTCAAAATGTACGAAATGCTTTG |
| | | GTAAAAAGCAAATCTCATTCATCTATTAAAACAATTGGAATTGGAGCTGGAGTTGG |
| | | AGCTGGAGGAGCTGGAGTGACAGGTTCTGTAGCAGTGAATAAGATTGTAAATAATA |
| | | CGATAGCAGAATTAAATCATGCAAAAATCACTGCGAAGGGAAATGTCGGAGTTATT |
| | | ACAGAGTCTGATGCGGTAATTGCTAATTATGCAGGAACAGTGTCTGGAGGGGCCCG |
| | | TGCAGCAATAGGAGCCTCAACCAGTGTGAATGAAATTACAGGATCTACAAAAGCAT |
| | | ATGTAAAAGATTCTACAGTGATTGCTAAAGAAGAAACAGATGATTATATTACTACT |
| | | CAAGGGCAAGTAGATAAAGTGGTAGATAAAGTATTCAAAAATCTTAATATTAACGA |
| | | AGACTTATCACAAAAAAGAAAAATAAGTAATAAAAAAGGATTTGTTACCAATAGTT |
| | | CAGCTACTCATACTTTAAAATCTTTATTGGCAAATGCCGCTGGTTCAGGACAAGCC |
| | | GGAGTGGCAGGAACTGTTAATATCAACAAGGTTTATGGAGAAACAGAAGCTCTTGT |
| | | AGAAAATTCTATATTAAATGCAAAACATTATTCTGTAAAGTCAGGAGATTACACGA |
| | | ATTCAATCGGAGTAGTAGGTTCTGTTGGTGTTGGTGGAAATGTAGGAGTAGGAGCT |
| | | TCTTCTGATACCAATATTATAAAAAGAAATACCAAGACAAGAGTTGGAAAAACTAC |
| | | AATGTCTGATGAAGGTTTCGGAGAAGAAGCTGAAATTACAGCAGATTCTAAGCAAG |
| | | GAATTTCCTCTTTTGGAGTCGGAGTCGCAGCAGCCGGGGTAGGAGCCGGAGTGGCA |
| | | GGAACCGTTTCCGCAAATCAATTTGCAGGAAAGACGGAAGTAGATGTGGAAGAATG |
| | | ATATTTTTACAACAATTACCAACAACAACAAACAACAAACAACATTACAATTACAT |
| | | TTACAATTACAATGGTGAGCAAGGGCGAGGAGCTGTTCACCGGGGTGGTGCCCATC |
| | | CTGGTCGAGCTGGACGGCGACGTAAACGGCCACAAGTTCAGCGTGTCCGGCGAGGG |
| | | CGAGGGCGATGCCACCTACGGCAAGCTGACCCTGAAGTTCATCTGCACCACCGGCA |
| | | AGCTGCCCGTGCCCTGGCCCACCCTCGTGACCACCCTGTCCTGGGGCGTGCAGTGC |
| | | TTCGCCCGCTACCCCGACCACATGAAGCAGCACGACTTCTTCAAGTCCGCCATGCC |
| | | CGAAGGCTACGTCCAGGAGCGCACCATCTTCTTCAAGGACGACGGCAACTACAAGA |
| | | CCCGCGCCGAGGTGAAGTTCGAGGGCGACACCCTGGTGAACCGCATCGAGCTGAAG |
| | | GGCATCGACTTCAAGGAGGACGGCAACATCCTGGGGCACAAGCTGGAGTACAACTA |
| | | CATCAGCGACAACGTCTATATCACCGCCGACAAGCAGAAGAACGGCATCAAGGCCA |
| | | ACTTCAAGATCCGCCACAACATCGAGGACGGCGGCGTGCAGCTCGCCGACCACTAC |
| | | CAGCAGAACACCCCCATCGGCGACGGCCCCGTGCTGCTGCCCGACAACCACTACCT |
| | | GAGCACCCAGTCCAAGCTGAGCAAAGACCCCAACGAGAAGCGCGATCACATGGTCC |
| | | TGCTGGAGTTCGTGACCGCCGCCGGGATCACTCTCGGCATGGACGAGCTGTACAAG |
| | | TAAAGAAATTCAATTAAGGAAATAAATTAAGGAAATACAAAAAGGGGGGTAGTCAT |
| | | TTGTATATAACTTTGTATGACTTTTCTCTTCTATTTTTTTGTATTTCCTCCCTTTC |
| | | CTTTTCTATTTGTATTTTTTTATCATTGCTTCCATTGAATTAAGAAGAATATATAT |
| | | GAATATTATAATAAAGAGAAAATGCCAAGAGATTAGCATTTTCATTCGATCATTAT |
| | | ATACATTTTTGATTTGAGATATTTTGTTTTTTTTTTTATTTGTTAATAATTTAAGG |
| | | ATAAATAGTTCACTAAAGAGAAGATAGAATCATAACAAATGAAATTTCTAATTCAG |
| | | ATTAGAAAACAAAGAATGAATATCAAGCGTTATAGTATGATTTTGAATACTCTAAA |
| | | AAAGGAAGGAGGAAGGCGGGGGAGAGAAAAACTTTTGGATATATTCATTCCGATTG |
| | | AATTGCAAATATATCAACGATAGAATCAATTCAATTCTGAATTGCAATAAGCGAGC |
| | | GGGCTCTCTCAAATAGAGATGAGCTGCTAGACTACGTCGAATAATCAATTCAATGA |
| | | TTCAAAAAAAACTAAGAGATGGATGAAATTATACAAGGAATCCTGGTTTCAAAGAA |
| | | AAGGAAAATGGGGATATGGCGAAATCGGTAGACGCTACGGACTTGATTGTATTGAG |
| | | CCTTGGTATGGAAACCTGCTAAGTGGTAACTTCCAAATTCAGAGAAACCCTGGAAT |
| | | GAAAAATGGGCAATCCTGAGCCAAATCCCTTTTTTGAAAAAACAAGTGGTTCTCAA |
| | | ACTAGAACCCAAAGGAAAAGGATAGGTGCAGAGACTCAATGGAAGCTGTTCTAACG |
| | | AATCGAAGTAATTACGTTGTGTTGGTAGTGGAACTCCCTCGAAATTATAGAAAGAA |
| | | GGGCTTTATACATCTAATACACACGTATAGATACTGACATAGCAAACGATTAATCA |
| | | TAGAACCCATATCATAATATAGGTTCTTTATTTTATTTTTTAAAATGAAATTAGGA |
| | | ATGATTATGAAATATAAAATTCTGAATTTTTTTTAGAATTATTGTGAATCCATTCC |
| | | AATCGAATATTGAGTAATCAAATCCTTCAATTCATTGTTTTGAGATCTTCAAAAAA |
| | | GTGGATTAATCGAACGAGGATAAAGAGAGAGTCCCATTCTACATGTCAATACTGAC |
| | | AACAATGAAATTTCTAGTAAAAGGAAAATCCGTCGACTTTATAAGTCGTGAGGGTT |
| | | CAAGTCCCTCTATCCCCAAACCCTCTTTTATTCCCTAACCATAGTAGTTATCCTTT |
| | | TTTTTCTTTTATCAATGGGTTTAAGATTCATTAGCTTTCTCATTCTACTCTTTCAC |
| | | AAAGGAGTGCTACGAGAACTCAATGAATCTTATGCTATTCATTAAATAGATGATTT |
| | | CTTTTTTATTTGATAGGATTACCCCGCCCATTTCCAAATTTAGAATGGAATACTTT |
| | | ATTGATTTTTTAGTCCCTTTAATTGACATAGATGCAAATACTCTAGTAGGATGATG |
| | | CACAAGAAAG |
| 21 | Prrn_{Nicotiana} | GGGCAACCCACTAGCATATCGAAATTCTAATTTTCTGTAGAGAAGTCCGTATTTTT |
| | alt | CCAATCAACTTCATTAAAAATTTGAATAGATCTAGATACACCTTGGTTGACACGAG |
| | | TATATAAGTCATGTTATACTGTTGAATAACAAGCCTTCCATTTTCTATTTTGATTT |
| | | GTAGAAAACTAGTGTGCTTGGGAGTCCCTGATGATTAAATAAACCAAGATTTTACC |
| | | ATGGCA |
| 22 | Trpsl6_{Nicotiana} | AAGAAATTCAATTAAGGAAATAAATTAAGGAAATACAAAAAGGGGGGTAGTCATTT |
| | alt | GTATATAACTTTGTATGACTTTTCTCTTCTATTTTTTTGTATTTCCTCCCTTTCCT |
| | | TTTCTATTTGTATTTTTTTATCATTGCTTCCATTGAATT |
| 23 | Right Flank_{Sorghum} | TTCATCCATAGATCCTTTACTCATATTTATTCAATCGGAATACTTATCGGAATACT |
| | alt | TAATCCAATGCAAAATTTTGCTTCGCGACTAGGTAGTCATAATCGAATTTGTATTT |
| | | TAGATGCAAATTCAATTAGTCTTTGGATACTAATCGCGAGAATGTATATTCTTCCT |
| | | CAATATGCTATTGAGAGGAAAAGGATTAAACCCTTTATAAGAACTAAAGTTTTCAT |
| | | CGGAATATGAATATAAAAAAACTTAAGGATGCCTTAAGTATATCATTTCAAATTCA |
| | | GTTATTAATAGAACGAATCACATTTTACCACTAAACTATACCCGCTACATGTAGAT |
| | | TATGATACCAATGCTACCCTTTGTCAAGGGTAGCCATTCGAGAAGGAGGCTAATTC |
| | | CACCTTATCGAATCAAAGGAGAAAGTTCATGGCGGTGGGCGATTGGTACTTCAATC |
| | | GCGGGTCTTTACTTTAGGATTTAGATAGCCCCTCTCTAGTCTGTAAAATACATCTC |
| | | TTCTTACCATACCAATAGCGTATGAACCAAATGTATGCATTTCGATTAGGATCTAT |
| | | TCTACGGTTATGACTACAAGGATCATTATTTGTAAGGACGTAAATGTGCCAGACTG |
| | | TTGTCTGGAATCGTTTAATTATTCCTACAATATATACTAAGAGATATAAAGGCAGT |
| | | ACAATCCCCTCCCTTTCTTCCTTTTCTTTTTTGTTCAGAATTGAACAAAGAAATTG |
| | | GGAAAGATGTTTTCTTCCTCCACGTATCATGAAGTGCGAGCCATAGGGAAGGAGTG |
| | | AGATGACTTTCACAAATTTATCATAGACTTCGTCTATCGCTTGAGAGAAGCAACAA |
| | | AGAGTAATAACTTAAAAAGAAAAACAGATACGAATCGACAGATTTACCTGATGAAA |
| | | ATTGACATCAGAGGACTCTGATGAGGATTCCTCAAACTCTTCATAAAGAGGATCCA |
| | | GAAAGTCTCTGGTTAGTCGAGACCCTCCATTTCCTAATTTCCTCTCTTCTTTTCCG |
| | | CTCAATTCTAGTTTATTAGATTCTTGTTTAAAAGAATCAAAGAAGATGAATAGAAC |
| | | TAAGA |
| 24 | PpsbA_{Nicotiana} | GGGCAACCCACTAGCATATCGAAATTCTAATTTTCTGTAGAGAAGTCCGTATTTTT |
| | | CCAATCAACTTCATTAAAAATTTGAATAGATCTAGATACACCTTGGTTGACACGAG |
| | | TATATAAGTCATGTTATACTGTTGAATAACAAGCCTTCCATTTTCTATTTTGATTT |
| | | GTAGAAAACTAGTGTGCTTGGGAGTCCCTGATGATTAAATAAACCAAGATTTTACC |
| | | ATGGCA |

### Equivalents and Scope

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of transforming a plant comprising
providing a nucleic acid material comprising
a first targeting sequence and a second targeting sequence,
a promoter sequence, and
an exogenous nucleic acid sequence; and
transforming a chloroplast in a plant cell with the nucleic acid material.

2. The method of claim 1, wherein the nucleic acid material further comprises a selection sequence.

3. The method of claim 1, further comprising expressing the exogenous nucleic acid sequence, wherein the expression occurs, at least in part, in a chloroplast.

4. The method of claim 1, wherein at least one of the first targeting sequence and second targeting sequence are directed to sequences located between chromosomal coordinates selected from trnI-trnA, trnM-trnG, rrn16-rps12/7, tscA-psac, trnV-trnA, rbcL-accD, rp132-trnL, 3′rps12/7-trnV, petA-psbJ, Trn16/V-16srrnA, trnfM-trnG, atpB-rbcL, trN-trnR, Ycf3-trnS, Rps7-ndhB, trnY-GUA-trnD-GUC, trnG-UCC-trnM-CAU, trnT-trnL and any combination thereof.

5-15. (canceled)

16. The method of claim 1, wherein after transformation step, a portion of the nucleic acid material is removed.

17. (canceled)

18. The method of claim 16, wherein the removal occurs, at least in part, through at least one of homologous recombination and site-specific recombination.

19. (canceled)

20. The method of claim 1, wherein the plant is millet or sorghum.

21. (canceled)

22. The method of claim 20, wherein the first targeting sequence and second targeting sequence are directed to a sequence located between trnG-UCC-trnM-CAU.

23. The method of claim 22, wherein the first and second targeting sequences have sequences at least 80% identical to SEQ ID NO: 1 and SEQ ID NO: 8 or 23, respectively.

24. (canceled)

25. The method of claim 20, wherein the first targeting sequence and second targeting sequence are directed to a sequence located between trnY-GUA-trnD-GUC or trnT-trnL.

26. The method of claim 25, wherein the first and second targeting sequences have sequences at least 80% identical to SEQ ID NO: 15 and SEQ ID NO: 16, respectively.

27. The method of claim 1, wherein the exogenous nucleic acid sequence encodes a peptide comprising a sequence that is at least 80% identical to a leukotoxin A (ltkA) protein according to Genbank: DQ672338, or a fragment or variant thereof.

28. The method of claim 27, wherein the exogenous nucleic acid sequence comprises a sequence encoding at least one region of ltkA selected from the group consisting of PL1, PL2, PL3, PL4, PL5, or a fragment or variant thereof.

29-30. (canceled)

31. A plant comprising
a nucleic acid material comprising
a first targeting sequence and a second targeting sequence,
a promoter sequence, and
at least one exogenous nucleic acid sequence, wherein at least one exogenous nucleic acid sequence is expressed, at least in part, in the chloroplast of the plant.

32. (canceled)

33. The plant of claim 31, wherein at least one of the first targeting sequence and second targeting sequence are directed to sequences located between chromosomal coordinates selected from trnI-trnA, trnM-trnG, rrn16-rps12/7, tscA-psac, trnV-trnA, rbcL-accD, rp132-trnL, 3′rps12/7-trnV, petA-psbJ, Trn16/V-16srrnA, trnfM-trnG, atpB-rbcL, trN-trnR, Ycf3-trnS, Rps7-ndhB, trnY-GUA-trnD-GUC, trnG-UCC-trnM-CAU, trnT-trnL and any combination thereof.

34-43. (canceled)

44. The plant of claim 31, wherein the plant is millet or sorghum.

45. (canceled)

46. The plant of claim 44, wherein the first targeting sequence and second targeting sequence are directed to a sequence located between trnG-UCC-trnM-CAU.

47. The plant of claim 46, wherein the first and second targeting sequences have sequences at least 80% identical to SEQ ID NO: 1 and SEQ ID NO: 8 or 23, respectively.

48. (canceled)

49. The plant of claim 44, wherein the first targeting sequence and second targeting sequence are directed to a sequence located between trnY-GUA-trnD-GUC or trnT-trnL.

50. The plant of claim 49, wherein the first and second targeting sequences have sequences at least 80% identical to SEQ ID NO: 15 and SEQ ID NO: 16, respectively.

51. The plant of claim 31, wherein the exogenous nucleic acid sequence encodes a peptide comprising a sequence that is at least 80% identical to a leukotoxin A (ltkA) protein) according to GenBank Ref: DQ672338, or a fragment or variant thereof.

52. The plant of claim 51, wherein the exogenous nucleic acid sequence comprises a sequence encoding at least one region of ltkA selected from the group consisting of PL1, PL2, PL3, PL4, PL5, or any a fragment or variant thereof.

53-62. (canceled)

63. A plant comprising
an exogenous nucleic acid sequence, wherein at least one exogenous nucleic acid sequence is expressed, at least in part, in the chloroplast of the plant.

64. (canceled)

65. The plant of claim 63, wherein the exogenous nucleic acid sequence is integrated in the chloroplast genome of the plant.

66. The plant of claim 65, wherein the exogenous nucleic acid sequence is stably integrated in the chloroplast of the plant.

67. A method of transforming a plant comprising
providing a nucleic acid material and a carrier, the nucleic acid material comprising
a first targeting sequence and a second targeting sequence,
a promoter sequence, and
an exogenous nucleic acid sequence; and
transforming a chloroplast in a plant cell with the nucleic acid material.

68. The method of claim 67, wherein the nucleic acid material is conjugated to the carrier.

69. The method of claim 68, wherein the carrier is a nanoparticle.

70. The method of claim 69, wherein the nanoparticle comprises a nanotube.

71-76. (canceled)

77. A kit comprising:
a nucleic acid material comprising
a first targeting sequence and a second targeting sequence,
a promoter sequence,
a selection sequence; and
at least one exogenous nucleic acid sequence; and
a nanoparticle carrier.

78-81. (canceled)

82. A method of administering an modified plant comprising an antigen to a non-human animal, the method comprising
administering an immunogenic composition according to claim 31 to a non-human animal.

83-89. (canceled)

90. A method of treating one or more symptoms of Fusobacterium infection in a non-human animal, the method comprising:
administering an immunogenic composition,
wherein the immunogenic composition comprises:
a plant comprising an exogenous nucleic acid sequence, wherein at least one exogenous nucleic acid sequence is expressed, at least in part, in the chloroplast of the plant; and
wherein the exogenous nucleic acid sequence encodes a peptide comprising sequence that is at least 80% identical to a leukotoxin A (ltkA) protein according to GenBank Ref: DQ672338, or a fragment or variant thereof.

91-105. (canceled)


SEQUENCE LISTING (54 sequences)

SEQ ID NO: 1 - DNA, 1340 bp, Artificial Sequence
  Description of Artificial Sequence Synthetic polynucleotide
agtatttagtaacccgatacaaaataaataaaaagaaaggcccttttttcgaaaaaattcggatttttatacacattaaaatgctattttcgttccgaattattacctattctctttcattattatgaaattctattgccattagaatattgattgatagacaattaataaaaagaaaactctaatagaaaatgaaacggtcgacccagacatagacggtcgacccgggtggatataccctataaaatataggccgtagcaagcgtagttcaatgtagcgagcgtagttcagtggtaaaacatctccttgccaaggagaagatacgggttcgattcccgtcgctcgccagcttaatttagtaaggtgctatgataaaaaattcagtttagttgactacttaacaacttctattaaattactatttaatattaaatgaatatgaaattcagtagttgttagtctagtactgtaccccttcctatcttatccttcctttgcacccgactcaaaaaaaagagtgcttcgagggcgcaaattcaactttctaagaaagttccctaaaccggggattcgccgagacaaacaagagacaaacggttttgaaagggggataggctatgcttttctttcatttttttttctgcctgctgaataaaaaaagggttggatatagccctctatcatatatatagaaatagaatagtccatttatacggactgctaagtgcggagacgggaatcgaacccgtgacctcaaggttatgagcctcgtgagctaccaaactgctctactccgctctgtagggccgaaaactggtggacgaaagaaaaaggttgaatacaagtctctaccatgtctagacaaacaaatggaatagtctttttatacagaatggagcgggtagcgggaatcgaacccgcatcgttagcttggaaggctaggggttatagtcgacgttggttgattatttttgacgtctctaattcaaaaccgaacatgaaattttgatttcattcggctcctttatggatattctcaccacttaacatctatgtcagcttttctgtctgaatggaaccaaagctctctgctttctagatgatccttatagagtaggagatagaaattctcctaaatatctatctaatctacttacttcgttccctaatttcattcaagagatcctgaggaaaagagttgggtttccaccgagctgaaacaatataatatactgatggttctagtaaaccaaaaccatcgttttttagctattgggcttccatttcctacaaaacaaaagaagatttagttacgattggaaataaacttttttgtatc

SEQ ID NO: 2 - DNA, 129 bp, Artificial Sequence
  Description of Artificial Sequence Synthetic polynucleotide
ggtaccccaaagctcccccgccgtcgttcaatgagaatggataagaggctcgtgggattgacgtgagggggcagggatggctatatttctgggagcgaactccgggcgaatacgaagcgcttggatacg

SEQ ID NO: 3 - DNA, 63 bp, Artificial Sequence
  Description of Artificial Sequence Synthetic oligonucleotide
gggagaccacaacggtttccctctagaaataattttgtttaactttaagaaggagatataccc

SEQ ID NO: 4 - DNA, 501 bp, Artificial Sequence
  Description of Artificial Sequence Synthetic polynucleotide
atgagcggcatcaaaagtaacgttcagaggacaaggaagaggatatcagattctaaaaaagttttaatgattttgggattgttgattaacactatgacggtgagggctaatgatacaatcgccgcgactgagaattttggaacaaaaatagaaaaaaaggataatgtttatgacattactacaaacaagattcaaggggagaacgcttttaacagttttaatagatttgctttaacagaaaataatatagcaaatctatattttggggaaaagaatagtacgggggtaaataatctttttaactttgtcaatggaaaaattgaagtagatgggattatcaacggaattcgagaaaataaaattggaggaaatttatatttcttaagctcggaagggatggcagtaggaaaaaatggagttatcaatgctggttcttttcattctattattccaaaacaagatgattttaagaaggctttggaagaagccaaacatggttaa

SEQ ID NO: 5 - DNA, 53 bp, Artificial Sequence
  Description of Artificial Sequence Synthetic oligonucleotide
agataacccaaataatgttttaaaattttaaaaataatgtaggaggaaaaatt

SEQ ID NO: 6 - DNA, 717 bp, Artificial Sequence
  Description of Artificial Sequence Synthetic polynucleotide
atgtccaagggcgaggagctgttcaccggcgtggtgcctatcctcgtggagctcgacggcgacgtgaacggccacaagttcagcgtgtccggcgagggcgagggcgacgccacctacggcaagctgaccctgaagttcatctgcaccaccggcaagctcccggtgccatggccaaccctggtgaccaccttcggctacggcctgcagtgcttcgccaggtaccccgaccacatgaagaggcacgacttcttcaagagcgccatgccagagggctacgtgcaggagaggaccatcttcttcaaggacgacggcaactacaagaccagggccgaggtgaagttcgagggcgacaccctggtgaacaggatcgagctgaagggcatcgacttcaaggaggacggcaacatcctgggccacaagctggagtacaactacaactcccacaacgtgtacatcatggccgacaagcagaagaacggcatcaaggtgaacttcaagatccgccacaacatcgaggacggctccgtgcagctggccgaccactaccagcagaacaccccaatcggcgacggcccggtgctcctccctgacaaccactacctcagctaccagtccgccctcagcaaggacccgaacgagaagagggaccacatggtgctgctggagttcgtgaccgccgccggcatcacccacggcatggacgagctctacaagtga

SEQ ID NO: 7 - DNA, 150 bp, Artificial Sequence
  Description of Artificial Sequence Synthetic polynucleotide
agaaattcaattaaggaaataaattaaggaaatacaaaaaggggggtagtcatttgtatataactttgtatgacttttctcttctatttttttgtatttcctccctttccttttctatttgtatttttttatcattgcttccattgaatt

SEQ ID NO: 8 - DNA, 1070 bp, Artificial Sequence
  Description of Artificial Sequence Synthetic polynucleotide
ttcatccatagatcctttactcatatttattcaatcggaatacttatcggaatacttaatccaatgcaaaattttgcttcgcgactacgtactcataatcgaatttgtattttagatgcaaattcaattagtctttggatactaatcgcgagaatgtatattcttcctcaatatgctattgagaggaaaaggattaaaccctttataagaactaaagttttcatcggaatatgaatataaaaaaacttaaggatgccttaagtatatcatttcaaattcagttattaatagaacgaatcacacttttaccactaaactatacccgctacatgtagattatgataccaatgctaccctttgtcaagggtagccattcgagaaggaggctaattccaccttatcgaatcaaaggagaaagttcatggcggtgggcgattggtacttcaatcgcgggtctttactttaggatttagatagcccctctctagtctgtaaaatacatctcttcttaccataccaatagcgtatgaaccaaatgtatgcatttcgattaggatctattctacggttatgactacaaggatcattatttgtaaggacgtaaatgtgccagactgttgtctggaatcgtttaattattcctacaatatatactaagagatataaaggcagtacaatcccctccctttcttccttttcttttttgttcagaattgaacaaagaaattgggaaagatgttttcttcctccacgtatcatgaagtgcgagccatagggaaggagtgagatgactttcacaaatttatcatagacttcgtctatcgcttgagagaagcaacaaagagtaataacttaaaaagaaaaacagatacgaatcgacagatttacctgatgaaaattgacatcagaggactctgatgaggattcctcaaactcttcataaagaggatccagaaagtctctggttagtcgagaccctccatttcctaatttcctctcttcttttccgctcaattctagtttattagattcttgtttaaaagaatcaaagaagatgaatagaactaaga

SEQ ID NO: 9 - DNA, 57 bp, Artificial Sequence
  Description of Artificial Sequence Synthetic oligonucleotide
tcgagtagaccttgttgttgtgaaaattcttaattcatgagttgtagggagggattt

SEQ ID NO: 10 - DNA, 975 bp, Artificial Sequence
  Description of Artificial Sequence Synthetic polynucleotide
atggtagcagttaataaaattacacaaaatacttctgcacatataaaaaatagtactcaaaatgtacgaaatgctttggtaaaaagcaaatctcattcatctattaaaacaattggaattggagctggagttggagctggaggagctggagtgacaggttctgtagcagtgaataagattgtaaataatacgatagcagaattaaatcatgcaaaaatcactgcgaagggaaatgtcggagttattacagagtctgatgcggtaattgctaattatgcaggaacagtgtctggaggggcccgtgcagcaataggagcctcaaccagtgtgaatgaaattacaggatctacaaaagcatatgtaaaagattctacagtgattgctaaagaagaaacagatgattatattactactcaagggcaagtagataaagtggtagataaagtattcaaaaatcttaatattaacgaagacttatcacaaaaaagaaaaataagtaataaaaaaggatttgttaccaatagttcagctactcatactttaaaatctttattggcaaatgccgctggttcaggacaagccggagtggcaggaactgttaatatcaacaaggtttatggagaaacagaagctcttgtagaaaattctatattaaatgcaaaacattattctgtaaagtcaggagattacacgaattcaatcggagtagtaggttctgttggtgttggtggaaatgtaggagtaggagcttcttctgataccaatattataaaaagaaataccaagacaagagttggaaaaactacaatgtctgatgaaggtttcggagaagaagctgaaattacagcagattctaagcaaggaatttcctcttttggagtcggagtcgcagcagccggggtaggagccggagtggcaggaaccgtttccgcaaatcaatttgcaggaaagacggaagtagatgtggaagaatga

SEQ ID NO: 11 - DNA, 90 bp, Artificial Sequence
  Description of Artificial Sequence Synthetic oligonucleotide
gaattaaccgatcgacgtgcaagcggacatttattttaaattcgataatttttgcaaaaacatttcgacatatttatttattttattatt

SEQ ID NO: 12 - DNA, 695 bp, Artificial Sequence
  Description of Artificial Sequence Synthetic polynucleotide
atggcctcctccgagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctgcttcatctacaaggtgaagttcatcggcgtgaacttcccctccgacggccccgtaatgcagaagaagaccatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcaccgagggccgccaccacctgttcctggtaccctaagagctccgtaa

SEQ ID NO: 13 - DNA, 66 bp, Artificial Sequence
  Description of Artificial Sequence Synthetic oligonucleotide
tatttttacaacaattaccaacaacaacaaacaacaaacaacattacaattacatttacaattaca

SEQ ID NO: 14 - DNA, 720 bp, Artificial Sequence
  Description of Artificial Sequence Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgtcctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagcgacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaa

SEQ ID NO: 15 - DNA, 1356 bp, Artificial Sequence
  Description of Artificial Sequence Synthetic polynucleotide
agttcaagtctggcgagagtaatattctacaactaacaactcatttactttgagaccgacccacttcctatctagaattttttttactagtcctttatattgcaatgtgtcaaccgtcaaatgctttggcaatttgcccggatcggatgaagcaatagaattttgaaccagacgttttgatcgttggttatccttcgtagtaataatatctcggggtttgcaacgaaaacttggtatatcaactatacgaccattaactaaaatatgtctatggttaactaattgccgggccccaggaatggttgaagccatacccaatcgaaaaagtatattatccaaacgcatttcaagtaattgtagtaaaacctgacctgtggatctttttgcttttccagcgatatgtacatatctaagtaattggcgttctgtcagaccataatgaaaacgcaatttctgtttttcttgaagacgaatacgatattgctcttttttcccagaatggaatttctttttctgattacttccggatttaggcgtttttctagtgagtcctggtaaagctcccagacggcgtattttttttaaacgaggccctcgataacgggacatgaagactcctttttttttattgaaatttcattttacacaataaatttcattctatttacattacaaaatacatcgaaattcaaactgaattaaactaaaggataagcagagtaaaatctactaaaagtaccacaaaaaatgaagtaccacaaaaaatggaatttcatcaacatccggattttttgtatatatattatttatttttatgctttgtatctagcaaaattgtaaggtagaacgacataaaagaccctggcttccccatttaatttagagaaaaagagaaattcttgttcatggaacatcgatagagaaaaaagccgactatcggatttgaaccgatgaccctcgcattacaaatgcgatgctctaacctctgagctaagtgggcttacataacagaaatagtgtaacaaatagaaatatatatagggaatctgtaaaatgtcagatcttaattattaatcttagttattaactagttcgaaattggaagttctacttagaattagtaaaagaattagtaaaaaaaatactagaatttcataaagataaaattagcttgatatgcttaactaaatgatattcttaaataggattctagaatttattgaactttctttttatttctctaattcgcaaatggatttttctattctaatagaatctattccaaattctatattgaatttgatttcagatattttcaatttgatatggctcggacgaataatctaatacatataa

SEQ ID NO: 16 - DNA, 1369 bp, Artificial Sequence
  Description of Artificial Sequence Synthetic polynucleotide
aagaagaatatatatgaatattataataaagagaaaatgccaagagattagcattttcattcgatcattatatacatttttgatttgagatattttgtttttttttttatttgttaataatttaaggataaatagttcactaaagagaagatagaatcataacaaatgaaatttctaattcagattagaaaacaaagaatgaatatcaagcgttatagtatgattttgaatactctaaaaaaggaaggaggaaggcgggggagagaaaaacttttggatatattcattccgattgaattgcaaatatatcaacgatagaatcaattcaattctgaattgcaataagcgagcgggctctctcaaatagagatgagctgctagactacgtcgaataatcaattcaatgattcaaaaaaaactaagagatggatgaaattatacaaggaatcctggtttcaaagaaaaggaaaatggggatatggcgaaatcggtagacgctacggacttgattgtattgagccttggtatggaaacctgctaagtggtaacttccaaattcagagaaaccctggaatgaaaaatgggcaatcctgagccaaatcccttttttgaaaaaacaagtggttctcaaactagaacccaaaggaaaaggataggtgcagagactcaatggaagctgttctaacgaatcgaagtaattacgttgtgttggtagtggaactccctcgaaattatagaaagaagggctttatacatctaatacacacgtatagatactgacatagcaaacgattaatcatagaacccatatcataatataggttctttattttattttttaaaatgaaattaggaatgattatgaaatataaaattctgaattttttttagaattattgtgaatccattccaatcgaatattgagtaatcaaatccttcaattcattgttttgagatcttcaaaaaagtggattaatcgaacgaggataaagagagagtcccattctacatgtcaatactgacaacaatgaaatttctagtaaaaggaaaatccgtcgactttataagtcgtgagggttcaagtccctctatccccaaaccctcttttattccctaaccatagtagttatccttttttttcttttatcaatgggtttaagattcattagctttctcattctactctttcacaaaggagtgctacgagaactcaatgaatcttatgctattcattaaatagatgatttcttttttatttgataggattaccccgcccatttccaaatttagaatggaatactttattgattttttagtccctttaattgacatagatgcaaatactctactaggatgatgcacaagaaag

SEQ ID NO: 17 - DNA, 4023 bp, Artificial Sequence
  Description of Artificial Sequence Synthetic polynucleotide
agtatttagtaacccgatacaaaataaataaaaagaaaggcccttttttcgaaaaaattcggatttttatacacattaaaatgctattttcgttccgaattattacctattctctttcattattatgaaattctattgccattagaatattgattgatagacaattaataaaaagaaaactctaatagaaaatgaaacggtcgacccagacatagacggtcgacccgggtggatataccctataaaatataggccgtagcaagcgtagttcaatgtagcgagcgtagttcagtggtaaaacatctccttgccaaggagaagatacgggttcgattcccgtcgctcgccagcttaatttagtaaggtgctatgataaaaaattcagtttagttgactacttaacaacttctattaaattactatttaatattaaatgaatatgaaattcagtagttgttagtctagtactgtaccccttcctatcttatccttcctttgcacccgactcaaaaaaaagagtgcttcgagggcgcaaattcaactttctaagaaagttccctaaaccggggattcgccgagacaaacaagagacaaacggttttgaaagggggataggctatgcttttctttcatttttttttctgcctgctgaataaaaaaagggttggatatagccctctatcatatatatagaaatagaatagtccatttatacggactgctaagtgcggagacgggaatcgaacccgtgacctcaaggttatgagcctcgtgagctaccaaactgctctactccgctctgtagggccgaaaactggtggacgaaagaaaaaggttgaatacaagtctctaccatgtctagacaaacaaatggaatagtctttttatacagaatggagcgggtagcgggaatcgaacccgcatcgttagcttggaaggctaggggttatagtcgacgttggttgattatttttgacgtctctaattcaaaaccgaacatgaaattttgatttcattcggctcctttatggatattctcaccacttaacatctatgtcagcttttctgtctgaatggaaccaaagctctctgctttctagatgatccttatagagtaggagatagaaattctcctaaatatctatctaatctacttacttcgttccctaatttcattcaagagatcctgaggaaaagagttgggtttccaccgagctgaaacaatataatatactgatggttctagtaaaccaaaaccatcgttttttagctattgggcttccatttcctacaaaacaaaagaagatttagttacgattggaaataaacttttttgtatcggtaccccaaagctcccccgccgtcgttcaatgagaatggataagaggctcgtgggattgacgtgagggggcagggatggctatatttctgggagcgaactccgggcgaatacgaagcgcttggatacggggagaccacaacggtttccctctagaaataattttgtttaactttaagaaggagatatacccatgagcggcatcaaaagtaacgttcagaggacaaggaagaggatatcagattctaaaaaagttttaatgattttgggattgttgattaacactatgacggtgagggctaatgatacaatcgccgcgactgagaattttggaacaaaaatagaaaaaaaggataatgtttatgacattactacaaacaagattcaaggggagaacgcttttaacagttttaatagatttgctttaacagaaaataatatagcaaatctatattttggggaaaagaatagtacgggggtaaataatctttttaactttgtcaatggaaaaattgaagtagatgggattatcaacggaattcgagaaaataaaattggaggaaatttatatttcttaagctcggaagggatggcagtaggaaaaaatggagttatcaatgctggttcttttcattctattattccaaaacaagatgattttaagaaggctttggaagaagccaaacatggttaaagataacccaaataatgttttaaaattttaaaaataatgtaggaggaaaaattatgtccaagggcgaggagctgttcaccggcgtggtgcctatcctcgtggagctcgacggcgacgtgaacggccacaagttcagcgtgtccggcgagggcgagggcgacgccacctacggcaagctgaccctgaagttcatctgcaccaccggcaagctcccggtgccatggccaaccctggtgaccaccttcggctacggcctgcagtgcttcgccaggtaccccgaccacatgaagaggcacgacttcttcaagagcgccatgccagagggctacgtgcaggagaggaccatcttcttcaaggacgacggcaactacaagaccagggccgaggtgaagttcgagggcgacaccctggtgaacaggatcgagctgaagggcatcgacttcaaggaggacggcaacatcctgggccacaagctggagtacaactacaactcccacaacgtgtacatcatggccgacaagcagaagaacggcatcaaggtgaacttcaagatccgccacaacatcgaggacggctccgtgcagctggccgaccactaccagcagaacaccccaatcggcgacggcccggtgctcctccctgacaaccactacctcagctaccagtccgccctcagcaaggacccgaacgagaagagggaccacatggtgctgctggagttcgtgaccgccgccggcatcacccacggcatggacgagctctacaagtgaagaaattcaattaaggaaataaattaaggaaatacaaaaaggggggtagtcatttgtatataactttgtatgacttttctcttctatttttttgtatttcctccctttccttttctatttgtatttttttatcattgcttccattgaattttcatccatagatcctttactcatatttattcaatcggaatacttatcggaatacttaatccaatgcaaaattttgcttcgcgactacgtactcataatcgaatttgtattttagatgcaaattcaattagtctttggatactaatcgcgagaatgtatattcttcctcaatatgctattgagaggaaaaggattaaaccctttataagaactaaagttttcatcggaatatgaatataaaaaaacttaaggatgccttaagtatatcatttcaaattcagttattaatagaacgaatcacacttttaccactaaactatacccgctacatgtagattatgataccaatgctaccctttgtcaagggtagccattcgagaaggaggctaattccaccttatcgaatcaaaggagaaagttcatggcggtgggcgattggtacttcaatcgcgggtctttactttaggatttagatagcccctctctagtctgtaaaatacatctcttcttaccataccaatagcgtatgaaccaaatgtatgcatttcgattaggatctattctacggttatgactacaaggatcattatttgtaaggacgtaaatgtgccagactgttgtctggaatcgtttaattattcctacaatatatactaagagatataaaggcagtacaatcccctccctttcttccttttcttttttgttcagaattgaacaaagaaattgggaaagatgttttcttcctccacgtatcatgaagtgcgagccatagggaaggagtgagatgactttcacaaatttatcatagacttcgtctatcgcttgagagaagcaacaaagagtaataacttaaaaagaaaaacagatacgaatcgacagatttacctgatgaaaattgacatcagaggactctgatgaggattcctcaaactcttcataaagaggatccagaaagtctctggttagtcgagaccctccatttcctaatttcctctcttcttttccgctcaattctagtttattagattcttgtttaaaagaatcaaagaagatgaatagaactaaga

SEQ ID NO: 18 - DNA, 4607 bp, Artificial Sequence
  Description of Artificial Sequence Synthetic polynucleotide
agtatttagtaacccgatacaaaataaataaaaagaaaggcccttttttcgaaaaaattcggatttttatacacattaaaatgctattttcgttccgaattattacctattctctttcattattatgaaattctattgccattagaatattgattgatagacaattaataaaaagaaaactctaatagaaaatgaaacggtcgacccagacatagacggtcgacccgggtggatataccctataaaatataggccgtagcaagcgtagttcaatgtagcgagcgtagttcagtggtaaaacatctccttgccaaggagaagatacgggttcgattcccgtcgctcgccagcttaatttagtaaggtgctatgataaaaaattcagtttagttgactacttaacaacttctattaaattactatttaatattaaatgaatatgaaattcagtagttgttagtctagtactgtaccccttcctatcttatccttcctttgcacccgactcaaaaaaaagagtgcttcgagggcgcaaattcaactttctaagaaagttccctaaaccggggattcgccgagacaaacaagagacaaacggttttgaaagggggataggctatgcttttctttcatttttttttctgcctgctgaataaaaaaagggttggatatagccctctatcatatatatagaaatagaatagtccatttatacggactgctaagtgcggagacgggaatcgaacccgtgacctcaaggttatgagcctcgtgagctaccaaactgctctactccgctctgtagggccgaaaactggtggacgaaagaaaaaggttgaatacaagtctctaccatgtctagacaaacaaatggaatagtctttttatacagaatggagcgggtagcgggaatcgaacccgcatcgttagcttggaaggctaggggttatagtcgacgttggttgattatttttgacgtctctaattcaaaaccgaacatgaaattttgatttcattcggctcctttatggatattctcaccacttaacatctatgtcagcttttctgtctgaatggaaccaaagctctctgctttctagatgatccttatagagtaggagatagaaattctcctaaatatctatctaatctacttacttcgttccctaatttcattcaagagatcctgaggaaaagagttgggtttccaccgagctgaaacaatataatatactgatggttctagtaaaccaaaaccatcgttttttagctattgggcttccatttcctacaaaacaaaagaagatttagttacgattggaaataaacttttttgtatcgggcaacccactagcatatcgaaattctaattttctgtagagaagtccgtatttttccaatcaacttcattaaaaatttgaatagatctacatacaccttggttgacacgagtatataagtcatgttatactgttgaataacaagccttccattttctattttgatttgtagaaaactagtgtgcttgggagtccctgatgattaaataaaccaagattttaccatggcatcgagtagaccttgttgttgtgaaaattcttaattcatgagttgtagggagggatttatggtagcagttaataaaattacacaaaatacttctgcacatataaaaaatagtactcaaaatgtacgaaatgctttggtaaaaagcaaatctcattcatctattaaaacaattggaattggagctggagttggagctggaggagctggagtgacaggttctgtagcagtgaataagattgtaaataatacgatagcagaattaaatcatgcaaaaatcactgcgaagggaaatgtcggagttattacagagtctgatgcggtaattgctaattatgcaggaacagtgtctggaggggcccgtgcagcaataggagcctcaaccagtgtgaatgaaattacaggatctacaaaagcatatgtaaaagattctacagtgattgctaaagaagaaacagatgattatattactactcaagggcaagtagataaagtggtagataaagtattcaaaaatcttaatattaacgaagacttatcacaaaaaagaaaaataagtaataaaaaaggatttgttaccaatagttcagctactcatactttaaaatctttattggcaaatgccgctggttcaggacaagccggagtggcaggaactgttaatatcaacaaggtttatggagaaacagaagctcttgtagaaaattctatattaaatgcaaaacattattctgtaaagtcaggagattacacgaattcaatcggagtagtaggttctgttggtgttggtggaaatgtaggagtaggagcttcttctgataccaatattataaaaagaaataccaagacaagagttggaaaaactacaatgtctgatgaaggtttcggagaagaagctgaaattacagcagattctaagcaaggaatttcctcttttggagtcggagtcgcagcagccggggtaggagccggagtggcaggaaccgtttccgcaaatcaatttgcaggaaagacggaagtagatgtggaagaatgagaattaaccgatcgacgtgcaagcggacatttattttaaattcgataatttttgcaaaaacatttcgacatatttatttattttattattatggcctcctccgagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctgcttcatctacaaggtgaagttcatcggcgtgaacttcccctccgacggccccgtaatgcagaagaagaccatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcaccgagggccgccaccacctgttcctggtaccctaagagctccgtaaaagaaattcaattaaggaaataaattaaggaaatacaaaaaggggggtagtcatttgtatataactttgtatgacttttctcttctatttttttgtatttcctccctttccttttctatttgtatttttttatcattgcttccattgaattttcatccatagatcctttactcatatttattcaatcggaatacttatcggaatacttaatccaatgcaaaattttgcttcgcgactacgtactcataatcgaatttgtattttagatgcaaattcaattagtctttggatactaatcgcgagaatgtatattcttcctcaatatgctattgagaggaaaaggattaaaccctttataagaactaaagttttcatcggaatatgaatataaaaaaacttaaggatgccttaagtatatcatttcaaattcagttattaatagaacgaatcacattttaccactaaactatacccgctacatgtagattatgataccaatgctaccctttgtcaagggtagccattcgagaaggaggctaattccaccttatcgaatcaaaggagaaagttcatggcggtgggcgattggtacttcaatcgcgggtctttactttaggatttagatagcccctctctagtctgtaaaatacatctcttcttaccataccaatagcgtatgaaccaaatgtatgcatttcgattaggatctattctacggttatgactacaaggatcattatttgtaaggacgtaaatgtgccagactgttgtctggaatcgtttaattattcctacaatatatactaagagatataaaggcagtacaatcccctccctttcttccttttcttttttgttcagaattgaacaaagaaattgggaaagatgttttcttcctccacgtatcatgaagtgcgagccatagggaaggagtgagatgactttcacaaatttatcatagacttcgtctatcgcttgagagaagcaacaaagagtaataacttaaaaagaaaaacagatacgaatcgacagatttacctgatgaaaattgacatcagaggactctgatgaggattcctcaaactcttcataaagaggatccagaaagtctctggttagtcgagaccctccatttcctaatttcctctcttcttttccgctcaattctagtttattagattcttgtttaaaagaatcaaagaagatgaatagaactaaga

SEQ ID NO: 19 - DNA, 5070 bp, Artificial Sequence
  Description of Artificial Sequence Synthetic polynucleotide
agtatttagtaacccgatacaaaataaataaaaagaaaggcccttttttcgaaaaaattcggatttttatacacattaaaatgctattttcgttccgaattattacctattctctttcattattatgaaattctattgccattagaatattgattgatagacaattaataaaaagaaaactctaatagaaaatgaaacggtcgacccagacatagacggtcgacccgggtggatataccctataaaatataggccgtagcaagcgtagttcaatgtagcgagcgtagttcagtggtaaaacatctccttgccaaggagaagatacgggttcgattcccgtcgctcgccagcttaatttagtaaggtgctatgataaaaaattcagtttagttgactacttaacaacttctattaaattactatttaatattaaatgaatatgaaattcagtagttgttagtctagtactgtaccccttcctatcttatccttcctttgcacccgactcaaaaaaaagagtgcttcgagggcgcaaattcaactttctaagaaagttccctaaaccggggattcgccgagacaaacaagagacaaacggttttgaaagggggataggctatgcttttctttcatttttttttctgcctgctgaataaaaaaagggttggatatagccctctatcatatatatagaaatagaatagtccatttatacggactgctaagtgcggagacgggaatcgaacccgtgacctcaaggttatgagcctcgtgagctaccaaactgctctactccgctctgtagggccgaaaactggtggacgaaagaaaaaggttgaatacaagtctctaccatgtctagacaaacaaatggaatagtctttttatacagaatggagcgggtagcgggaatcgaacccgcatcgttagcttggaaggctaggggttatagtcgacgttggttgattatttttgacgtctctaattcaaaaccgaacatgaaattttgatttcattcggctcctttatggatattctcaccacttaacatctatgtcagcttttctgtctgaatggaaccaaagctctctgctttctagatgatccttatagagtaggagatagaaattctcctaaatatctatctaatctacttacttcgttccctaatttcattcaagagatcctgaggaaaagagttgggtttccaccgagctgaaacaatataatatactgatggttctagtaaaccaaaaccatcgttttttagctattgggcttccatttcctacaaaacaaaagaagatttagttacgattggaaataaacttttttgtatcggtaccccaaagctcccccgccgtcgttcaatgagaatggataagaggctcgtgggattgacgtgagggggcagggatggctatatttctgggagcgaactccgggcgaatacgaagcgcttggatacggggagaccacaacggtttccctctagaaataattttgtttaactttaagaaggagatatacccatgagcggcatcaaaagtaacgttcagaggacaaggaagaggatatcagattctaaaaaagttttaatgattttgggattgttgattaacactatgacggtgagggctaatgatacaatcgccgcgactgagaattttggaacaaaaatagaaaaaaaggataatgtttatgacattactacaaacaagattcaaggggagaacgcttttaacagttttaatagatttgctttaacagaaaataatatagcaaatctatattttggggaaaagaatagtacgggggtaaataatctttttaactttgtcaatggaaaaattgaagtagatgggattatcaacggaattcgagaaaataaaattggaggaaatttatatttcttaagctcggaagggatggcagtaggaaaaaatggagttatcaatgctggttcttttcattctattattccaaaacaagatgattttaagaaggctttggaagaagccaaacatggttaatcgagtagaccttgttgttgtgaaaattcttaattcatgagttgtagggagggatttatggtagcagttaataaaattacacaaaatacttctgcacatataaaaaatagtactcaaaatgtacgaaatgctttggtaaaaagcaaatctcattcatctattaaaacaattggaattggagctggagttggagctggaggagctggagtgacaggttctgtagcagtgaataagattgtaaataatacgatagcagaattaaatcatgcaaaaatcactgcgaagggaaatgtcggagttattacagagtctgatgcggtaattgctaattatgcaggaacagtgtctggaggggcccgtgcagcaataggagcctcaaccagtgtgaatgaaattacaggatctacaaaagcatatgtaaaagattctacagtgattgctaaagaagaaacagatgattatattactactcaagggcaagtagataaagtggtagataaagtattcaaaaatcttaatattaacgaagacttatcacaaaaaagaaaaataagtaataaaaaaggatttgttaccaatagttcagctactcatactttaaaatctttattggcaaatgccgctggttcaggacaagccggagtggcaggaactgttaatatcaacaaggtttatggagaaacagaagctcttgtagaaaattctatattaaatgcaaaacattattctgtaaagtcaggagattacacgaattcaatcggagtagtaggttctgttggtgttggtggaaatgtaggagtaggagcttcttctgataccaatattataaaaagaaataccaagacaagagttggaaaaactacaatgtctgatgaaggtttcggagaagaagctgaaattacagcagattctaagcaaggaatttcctcttttggagtcggagtcgcagcagccggggtaggagccggagtggcaggaaccgtttccgcaaatcaatttgcaggaaagacggaagtagatgtggaagaatgatatttttacaacaattaccaacaacaacaaacaacaaacaacattacaattacatttacaattacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgtcctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagcgacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaaagaaattcaattaaggaaataaattaaggaaatacaaaaaggggggtagtcatttgtatataactttgtatgacttttctcttctatttttttgtatttcctccctttccttttctatttgtatttttttatcattgcttccattgaattttcatccatagatcctttactcatatttattcaatcggaatacttatcggaatacttaatccaatgcaaaattttgcttcgcgactacgtactcataatcgaatttgtattttagatgcaaattcaattagtctttggatactaatcgcgagaatgtatattcttcctcaatatgctattgagaggaaaaggattaaaccctttataagaactaaagttttcatcggaatatgaatataaaaaaacttaaggatgccttaagtatatcatttcaaattcagttattaatagaacgaatcacattttaccactaaactatacccgctacatgtagattatgataccaatgctaccctttgtcaagggtagccattcgagaaggaggctaattccaccttatcgaatcaaaggagaaagttcatggcggtgggcgattggtacttcaatcgcgggtctttactttaggatttagatagcccctctctagtctgtaaaatacatctcttcttaccataccaatagcgtatgaaccaaatgtatgcatttcgattaggatctattctacggttatgactacaaggatcattatttgtaaggacgtaaatgtgccagactgttgtctggaatcgtttaattattcctacaatatatactaagagatataaaggcagtacaatcccctccctttcttccttttcttttttgttcagaattgaacaaagaaattgggaaagatgttttcttcctccacgtatcatgaagtgcgagccatagggaaggagtgagatgactttcacaaatttatcatagacttcgtctatcgcttgagagaagcaacaaagagtaataacttaaaaagaaaaacagatacgaatcgacagatttacctgatgaaaattgacatcagaggactctgatgaggattcctcaaactcttcataaagaggatccagaaagtctctggttagtcgagaccctccatttcctaatttcctctcttcttttccgctcaattctagtttattagattcttgtttaaaagaatcaaagaagatgaatagaactaaga

SEQ ID NO: 20 - DNA, 5386 bp, Artificial Sequence
  Description of Artificial Sequence Synthetic polynucleotide
agttcaagtctggcgagagtaatattctacaactaacaactcatttactttgagaccgacccacttcctatctagaattttttttactagtcctttatattgcaatgtgtcaaccgtcaaatgctttggcaatttgcccggatcggatgaagcaatagaattttgaaccagacgttttgatcgttggttatccttcgtagtaataatatctcggggtttgcaacgaaaacttggtatatcaactatacgaccattaactaaaatatgtctatggttaactaattgccgggccccaggaatggttgaagccatacccaatcgaaaaagtatattatccaaacgcatttcaagtaattgtagtaaaacctgacctgtggatctttttgcttttccagcgatatgtacatatctaagtaattggcgttctgtcagaccataatgaaaacgcaatttctgtttttcttgaagacgaatacgatattgctcttttttcccagaatggaatttctttttctgattacttccggatttaggcgtttttctagtgagtcctggtaaagctcccagacggcgtattttttttaaacgaggccctcgataacgggacatgaagactcctttttttttattgaaatttcattttacacaataaatttcattctatttacattacaaaatacatcgaaattcaaactgaattaaactaaaggataagcagagtaaaatctactaaaagtaccacaaaaaatgaagtaccacaaaaaatggaatttcatcaacatccggattttttgtatatatattatttatttttatgctttgtatctagcaaaattgtaaggtagaacgacataaaagaccctggcttccccatttaatttagagaaaaagagaaattcttgttcatggaacatcgatagagaaaaaagccgactatcggatttgaaccgatgaccctcgcattacaaatgcgatgctctaacctctgagctaagtgggcttacataacagaaatagtgtaacaaatagaaatatatatagggaatctgtaaaatgtcagatcttaattattaatcttagttattaactagttcgaaattggaagttctacttagaattagtaaaagaattagtaaaaaaaatactagaatttcataaagataaaattagcttgatatgcttaactaaatgatattcttaaataggattctagaatttattgaactttctttttatttctctaattcgcaaatggatttttctattctaatagaatctattccaaattctatattgaatttgatttcagatattttcaatttgatatggctcggacgaataatctaatacatataaggtaccccaaagctcccccgccgtcgttcaatgagaatggataagaggctcgtgggattgacgtgagggggcagggatggctatatttctgggagcgaactccgggcgaatacgaagcgcttggatacggggagaccacaacggtttccctctagaaataattttgtttaactttaagaaggagatatacccatgagcggcatcaaaagtaacgttcagaggacaaggaagaggatatcagattctaaaaaagttttaatgattttgggattgttgattaacactatgacggtgagggctaatgatacaatcgccgcgactgagaattttggaacaaaaatagaaaaaaaggataatgtttatgacattactacaaacaagattcaaggggagaacgcttttaacagttttaatagatttgctttaacagaaaataatatagcaaatctatattttggggaaaagaatagtacgggggtaaataatctttttaactttgtcaatggaaaaattgaagtagatgggattatcaacggaattcgagaaaataaaattggaggaaatttatatttcttaagctcggaagggatggcagtaggaaaaaatggagttatcaatgctggttcttttcattctattattccaaaacaagatgattttaagaaggctttggaagaagccaaacatggttaatcgagtagaccttgttgttgtgaaaattcttaattcatgagttgtagggagggatttatggtagcagttaataaaattacacaaaatacttctgcacatataaaaaatagtactcaaaatgtacgaaatgctttggtaaaaagcaaatctcattcatctattaaaacaattggaattggagctggagttggagctggaggagctggagtgacaggttctgtagcagtgaataagattgtaaataatacgatagcagaattaaatcatgcaaaaatcactgcgaagggaaatgtcggagttattacagagtctgatgcggtaattgctaattatgcaggaacagtgtctggaggggcccgtgcagcaataggagcctcaaccagtgtgaatgaaattacaggatctacaaaagcatatgtaaaagattctacagtgattgctaaagaagaaacagatgattatattactactcaagggcaagtagataaagtggtagataaagtattcaaaaatcttaatattaacgaagacttatcacaaaaaagaaaaataagtaataaaaaaggatttgttaccaatagttcagctactcatactttaaaatctttattggcaaatgccgctggttcaggacaagccggagtggcaggaactgttaatatcaacaaggtttatggagaaacagaagctcttgtagaaaattctatattaaatgcaaaacattattctgtaaagtcaggagattacacgaattcaatcggagtagtaggttctgttggtgttggtggaaatgtaggagtaggagcttcttctgataccaatattataaaaagaaataccaagacaagagttggaaaaactacaatgtctgatgaaggtttcggagaagaagctgaaattacagcagattctaagcaaggaatttcctcttttggagtcggagtcgcagcagccggggtaggagccggagtggcaggaaccgtttccgcaaatcaatttgcaggaaagacggaagtagatgtggaagaatgatatttttacaacaattaccaacaacaacaaacaacaaacaacattacaattacatttacaattacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgtcctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagcgacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaaagaaattcaattaaggaaataaattaaggaaatacaaaaaggggggtagtcatttgtatataactttgtatgacttttctcttctatttttttgtatttcctccctttccttttctatttgtatttttttatcattgcttccattgaattaagaagaatatatatgaatattataataaagagaaaatgccaagagattagcattttcattcgatcattatatacatttttgatttgagatattttgtttttttttttatttgttaataatttaaggataaatagttcactaaagagaagatagaatcataacaaatgaaatttctaattcagattagaaaacaaagaatgaatatcaagcgttatagtatgattttgaatactctaaaaaaggaaggaggaaggcgggggagagaaaaacttttggatatattcattccgattgaattgcaaatatatcaacgatagaatcaattcaattctgaattgcaataagcgagcgggctctctcaaatagagatgagctgctagactacgtcgaataatcaattcaatgattcaaaaaaaactaagagatggatgaaattatacaaggaatcctggtttcaaagaaaaggaaaatggggatatggcgaaatcggtagacgctacggacttgattgtattgagccttggtatggaaacctgctaagtggtaacttccaaattcagagaaaccctggaatgaaaaatgggcaatcctgagccaaatcccttttttgaaaaaacaagtggttctcaaactagaacccaaaggaaaaggataggtgcagagactcaatggaagctgttctaacgaatcgaagtaattacgttgtgttggtagtggaactccctcgaaattatagaaagaagggctttatacatctaatacacacgtatagatactgacatagcaaacgattaatcatagaacccatatcataatataggttctttattttattttttaaaatgaaattaggaatgattatgaaatataaaattctgaattttttttagaattattgtgaatccattccaatcgaatattgagtaatcaaatccttcaattcattgttttgagatcttcaaaaaagtggattaatcgaacgaggataaagagagagtcccattctacatgtcaatactgacaacaatgaaatttctagtaaaaggaaaatccgtcgactttataagtcgtgagggttcaagtccctctatccccaaaccctcttttattccctaaccatagtagttatccttttttttcttttatcaatgggtttaagattcattagctttctcattctactctttcacaaaggagtgctacgagaactcaatgaatcttatgctattcattaaatagatgatttcttttttatttgataggattaccccgcccatttccaaatttagaatggaatactttattgattttttagtccctttaattgacatagatgcaaatactctactaggatgatgcacaagaaag

SEQ ID NO: 21 - DNA, 230 bp, Artificial Sequence
  Description of Artificial Sequence Synthetic polynucleotide
gggcaacccactagcatatcgaaattctaattttctgtagagaagtccgtatttttccaatcaacttcattaaaaatttgaatagatctacatacaccttggttgacacgagtatataagtcatgttatactgttgaataacaagccttccattttctattttgatttgtagaaaactagtgtgcttgggagtccctgatgattaaataaaccaagattttaccatggca

SEQ ID NO: 22 - DNA, 151 bp, Artificial Sequence
  Description of Artificial Sequence Synthetic polynucleotide
aagaaattcaattaaggaaataaattaaggaaatacaaaaaggggggtagtcatttgtatataactttgtatgacttttctcttctatttttttgtatttcctccctttccttttctatttgtatttttttatcattgcttccattgaatt

SEQ ID NO: 23 - DNA, 1069 bp, Artificial Sequence
  Description of Artificial Sequence Synthetic polynucleotide
ttcatccatagatcctttactcatatttattcaatcggaatacttatcggaatacttaatccaatgcaaaattttgcttcgcgactacgtactcataatcgaatttgtattttagatgcaaattcaattagtctttggatactaatcgcgagaatgtatattcttcctcaatatgctattgagaggaaaaggattaaaccctttataagaactaaagttttcatcggaatatgaatataaaaaaacttaaggatgccttaagtatatcatttcaaattcagttattaatagaacgaatcacattttaccactaaactatacccgctacatgtagattatgataccaatgctaccctttgtcaagggtagccattcgagaaggaggctaattccaccttatcgaatcaaaggagaaagttcatggcggtgggcgattggtacttcaatcgcgggtctttactttaggatttagatagcccctctctagtctgtaaaatacatctcttcttaccataccaatagcgtatgaaccaaatgtatgcatttcgattaggatctattctacggttatgactacaaggatcattatttgtaaggacgtaaatgtgccagactgttgtctggaatcgtttaattattcctacaatatatactaagagatataaaggcagtacaatcccctccctttcttccttttcttttttgttcagaattgaacaaagaaattgggaaagatgttttcttcctccacgtatcatgaagtgcgagccatagggaaggagtgagatgactttcacaaatttatcatagacttcgtctatcgcttgagagaagcaacaaagagtaataacttaaaaagaaaaacagatacgaatcgacagatttacctgatgaaaattgacatcagaggactctgatgaggattcctcaaactcttcataaagaggatccagaaagtctctggttagtcgagaccctccatttcctaatttcctctcttcttttccgctcaattctagtttattagattcttgtttaaaagaatcaaagaagatgaatagaactaaga

SEQ ID NO: 24 - DNA, 230 bp, Artificial Sequence; identical to SEQ ID NO: 21
  Description of Artificial Sequence Synthetic polynucleotide

SEQ ID NO: 25 - DNA, 30 bp, Artificial Sequence
  Description of Artificial Sequence Synthetic primer
gttacgattggaaataaacttttttgtatc

SEQ ID NO: 26 - DNA, 32 bp, Artificial Sequence
  Description of Artificial Sequence Synthetic primer
gaataaatatgagtaaaggatctatggatgaa

SEQ ID NO: 27 - DNA, 29 bp, Artificial Sequence
  Description of Artificial Sequence Synthetic primer
ggctcggacgaataatctaatacatataa

SEQ ID NO: 28 - DNA, 39 bp, Artificial Sequence
  Description of Artificial Sequence Synthetic primer
cattttctctttattataatattcatatatattcttctt

SEQ ID NO: 29 - DNA, 23 bp, Artificial Sequence
  Description of Artificial Sequence Synthetic primer
gatgggattatcaacggaattcg

SEQ ID NO: 30 - DNA, 32 bp, Artificial Sequence
  Description of Artificial Sequence Synthetic primer
gtagcagttaataaaattacacaaaatacttc

SEQ ID NO: 31 - DNA, 21 bp, Artificial Sequence
  Description of Artificial Sequence Synthetic primer
aacccgcaaaaccccagacta

SEQ ID NO: 32 - DNA, 28 bp, Artificial Sequence
  Description of Artificial Sequence Synthetic primer
ccgagcttaagaaatataaatttcctcc

SEQ ID NO: 33 - DNA, 26 bp, Artificial Sequence
  Description of Artificial Sequence Synthetic primer
gatttgctttttaccaaagcatttcg

SEQ ID NO: 34 - DNA, 21 bp, Artificial Sequence
  Description of Artificial Sequence Synthetic primer
tacaggtcgggctcatggaac

SEQ ID NO: 35 - DNA, 37 bp, Artificial Sequence
  Description of Artificial Sequence Synthetic primer
gttttaatagatttgctttaacagaaaataatatagc

SEQ ID NO: 36 - DNA, 29 bp, Artificial Sequence
  Description of Artificial Sequence Synthetic primer
ggatctacaaaagcatatgtaaaagattc

SEQ ID NO: 37 - DNA, 22 bp, Artificial Sequence
  Description of Artificial Sequence Synthetic primer
tgagagcagacaaatcactcaa

SEQ ID NO: 38 - DNA, 30 bp, Artificial Sequence
  Description of Artificial Sequence Synthetic primer
ccattgacaaagttaaaaagattatttacc

SEQ ID NO: 39 - DNA, 25 bp, Artificial Sequence
  Description of Artificial Sequence Synthetic primer
actttatctacttgcccttgagtag

SEQ ID NO: 40 - DNA, 22 bp, Artificial Sequence
  Description of Artificial Sequence Synthetic primer
aagagctgtgagaggcaaataa

SEQ ID NO: 41 - DNA, 28 bp, Artificial Sequence
  Description of Artificial Sequence Synthetic probe
ctatattttggggaaaagaatagtacgg

SEQ ID NO: 42 - DNA, 26 bp, Artificial Sequence
  Description of Artificial Sequence Synthetic probe
cagtgattgctaaagaagaaacagat

SEQ ID NO: 43 - DNA, 30 bp, Artificial Sequence
  Description of Artificial Sequence Synthetic probe
ccttaacttactggtgttgatgctcctctc

SEQ ID NO: 44 - DNA, 30 bp, Artificial Sequence
  Description of Artificial Sequence Synthetic probe
cttctatgatgaatgcttaagaaaatatgg

SEQ ID NO: 45 - DNA, 30 bp, Artificial Sequence
  Description of Artificial Sequence Synthetic primer
ggtagctattctgaattctcttatttcttg

SEQ ID NO: 46 - DNA, 38 bp, Artificial Sequence
  Description of Artificial Sequence Synthetic primer
tgtaaaacgacggccagtcaaagaccccaacgagaagc

SEQ ID NO: 47 - DNA, 22 bp, Artificial Sequence
  Description of Artificial Sequence Synthetic primer
gctaggtgaacgggaaaatacg

SEQ ID NO: 48 - DNA, 38 bp, Artificial Sequence
  Description of Artificial Sequence Synthetic primer
caggaaacagctatgaccacgttacttttgatgccgct

SEQ ID NO: 49 - DNA, 24 bp, Artificial Sequence
  Description of Artificial Sequence Synthetic primer
gtttggtaatggttctctatgctc

SEQ ID NO: 50 - DNA, 34 bp, Artificial Sequence
  Description of Artificial Sequence Synthetic primer
attattttctgttaaagcaaatctattaaaactg

SEQ ID NO: 51 - DNA, 52 bp, Artificial Sequence
  Description of Artificial Sequence Synthetic oligonucleotide
aggcacatataaaaaatagtactcaaaatgtacgaaatgctttggtaaaaag

SEQ ID NO: 52 - DNA, 57 bp, Artificial Sequence
  Description of Artificial Sequence Synthetic oligonucleotide
tgcacatataaaaaatagtactcaaaatgtacgaaatgctttggtaaaaagcaaatc

SEQ ID NO: 53 - DNA, 22 bp, Artificial Sequence
  Description of Artificial Sequence Synthetic oligonucleotide
aataatctttttaactttgtca

SEQ ID NO: 54 - DNA, 16 bp, Artificial Sequence
  Description of Artificial Sequence Synthetic oligonucleotide
agggcaagtagataaa